(19) United States
(12) Patent Application Publication
(10) Pub. No.: US 2026/0041660 A1
(43) Pub. Date: Feb. 12, 2026

(54) Compositions And Methods for Treating Acute Respiratory Distress Syndrome (ARDS) And Inflammatory Disorders Caused by Corona Viruses and Other Respiratory Pathogens or Agents That Mediate Pulmonary Injury, Inflammation or Acute Respiratory Distress, And Related Compositions and Methods for Treating and Preventing Human SARS Coronavirus Infection, COVID-19 Disease and Related Symptoms

(71) Applicants: Richard L. CHANG, Laguna Woods, CA (US); Ben Y. Chang, Los Angeles, CA (US)

(72) Inventors: Richard L. CHANG, Laguna Woods, CA (US); Ben Y. Chang, Los Angeles, CA (US)

(21) Appl. No.: 19/362,286

(22) Filed: Oct. 17, 2025

Related U.S. Application Data
(63) Continuation of application No. 19/055,441, filed on Feb. 17, 2025, now abandoned.
Application No. 19/055,441 is a continuation of application No. 18/759,599, filed on Jun. 28, 2024, now abandoned.
Application No. 18/759,599 is a continuation of application No. 18/388,597, filed on Nov. 10, 2023, now abandoned.
Application No. 18/388,597 is a continuation of application No. 17/928,235, now abandoned.
(60) Provisional application No. 63/031,551, filed on May 28, 2020.

(51) Int. Cl. A61K 31/23; A61K 45/06; A61P 11/00; A61P 29/00; A61P 31/14

(52) U.S. Cl. CPC A61K 31/23; A61K 45/06; A61P 11/00; A61P 29/00; A61P 31/14

(57) ABSTRACT

Methods and compositions containing a phorbol ester or derivative of a phorbol ester are provided for prevention and treatment of sudden acute respiratory syndrome (SARS) coronavirus infection, including SARS-COV-2 infection and related COVID-19 disease. Also provided are methods and compositions for preventing and treating acute inflammatory conditions and related pathogenic injuries, including Acute Respiratory Distress Syndrome (ARDS) and cytokine storm syndrome (CSS) seen in severe SARS-COV-2/COVID-19 cases.

### TECHNICAL FIELD

The instant invention relates to therapeutic and prophylactic compositions and methods for treating pulmonary viral infections and related inflammatory and immunologic disease conditions of the lungs and other organs mediated by viral infection and other agents. In certain embodiments the invention relates to methods and compositions for treating Acute Respiratory Distress Syndrome (ARDS) or Severe Acute Respiratory Syndrome (SARS) caused by a viral pathogen, bacterial pathogen, a caustic agent, trauma, burn or other source of pulmonary or immune system injury or Inflammatory injury or stimulus. In more specific embodiments the invention relates to compositions and methods for treating and preventing pulmonary viral infections and associated pulmonary and inflammatory disease symptoms, ARDS/SARS caused by a SARS coronavirus or other respiratory virus.

### BACKGROUND OF THE INVENTION

Acute Respiratory Distress Syndrome (ARDS) (aka “adult respiratory distress syndrome”) is a serious pathological condition that often culminates in pulmonary failure, hospitalization and critical care. ARDS can be triggered by a variety of pulmonary and non-pulmonary causes, such as viral or bacterial pneumonia, aspiration of gastric content, inhalation of hot gasses or caustic agents, lung contusion or other pulmonary trauma, sepsis, acute pancreatitis and other causes. The most compelling, current cause of ARDS has emerged from the ongoing COVID-19 disease pandemic, caused by the SARS coronavirus SARS-COV-2.

Coronaviruses are enveloped, positive-sense single-stranded RNA viruses. They have the largest genomes (26-32 kb) among known RNA viruses. Coronaviruses infect a wide range of avian and mammalian species, including humans. Of the six known human coronaviruses, four of them (HCoV-OC43, HCOV-229E, HCoV-HKUl and HCoV-NL63) have circulated annually in human populations for centuries, and generally cause mild respiratory diseases, although severity can be greater in infants, elderly, and the immunocompromised.

In contrast, several recent coronavirus outbreaks have caused severe health and economic impacts among humans. These “novel” coronaviruses have all elicited ARDS in severe cases (specifically referred to as “Severe Acute Respiratory Syndrome” (SARS) in the context of coronavirus disease. Coronaviruses capable of causing ARDS/SARS include the original “SARS” virus (SARS-COV), which emerged in Guangdong China in 2002, and the COVID-19 (SARS-COV-2) coronavirus first identified in emerged in Wu Han City (aka “COVID-19 virus”), as well as the Middle East Respiratory Syndrome (MERS) coronavirus (MERS-COV). All SARS/MERS coronaviruses elicit ARDS as a proximal cause of the most severe disease cases involving debilitating respiratory tract infection.

Each of the SARS coronaviruses (SARS-COV, MERS-COV, and SARS-COV-2) was zoonotically transferred from other mammalian species to humans within the last 20 years, and has caused outbreaks with high case-fatality rates. Horseshoe bats are considered the primary reservoirs for these novel coronaviruses, and the intermediate hosts which transmitted the virus to humans have been identified as the masked palm civet for SARS-CoV, and the dromedary camel for MERS-COV. In the case of SARS-COV-2, a recent metagenomics study strongly indicates this newest SARS coronavirus was transmitted to the human population from the Malayan pangolin (Manis javanica), as an object of smuggling/trade in the Huanan wet market in Wuhan [Lam et al., 2020). The high pathogenicity and airborne transmissibility of SARS-COV and MERS-COV raised concerns about the potential for another coronavirus pandemic many years before current COVID-19 pandemic struck.

Since December 2019 the COVID-19 outbreak has continued to spread rapidly among human populations worldwide. The COVID-19 virus is highly contagious through airborne (expirated droplets) and contact transmission, and is now known to be transmissible by pre-symptomatic and asymptomatic carriers. On Mar. 11, 2020, the World Health Organization (WHO) declared the COVID-19 outbreak a pandemic. As of May 17, 2020 there have been over 4.75 million COVID-19 cases identified, accounting for more than 314,000 deaths in approximately 213 countries.

The clinical course of COVID-19 pneumonia exhibits a broad spectrum of severity and progression patterns. In some patients, dyspnea (shortness of breath) develops within a median of 8 days after the onset of illness (range of 5-13 days), while in others, respiratory distress may be absent. Roughly 3-30% of patients require admission to intensive care.

Severely ill patients may exhibit rapid progression to multiple organ dysfunction and even death. Those who present with shortness of breath and hypoxemia can quickly progress into acute respiratory distress syndrome (ARDS), severe sepsis with shock, and even multiple organ dysfunction within a matter of days, usually about one week. ARDS has been documented in roughly 15-30% of hospitalized patients with COVID-19, appearing on average 8 days after symptoms onset (Tu et al., 2020).

While COVID-19 disease may be moderate or even asymptomatic in a majority of people, about 20% of infected subjects develop severe symptoms. COVID-19 morbidity and mortality are particularly high among the elderly and in subjects with underlying pulmonary disease, heart disease, diabetes, cancer or other serious health conditions. A minority of patients who present with ARDS/SARS develop fatal cases, culminating in respiratory failure often attended by septic shock and multi-organ failure.

Pathogenesis of ARDS/SARS involves inflammation of the lung parenchyma, infiltration of neutrophils into pulmonary alveolar airspaces, oxidative stress, disruption of endothelial and epithelial barriers, damage to the epithelial lining and subsequent lung fibrosis, among other inflammatory, immunologic and tissue/organ injuries.

Despite our considerable knowledge regarding the mechanisms and pathogenesis of ARDS, more than 20 years of intensive clinical research has failed to yield effective treatments to prevent ARDS or substantially reduce its mortality (Fanelli et al., 2013). First line ARDS treatment remains primarily supportive, consisting of patient oxygenation or ventilation and tight control over patient fluid balance.

In the case of COVID-19 disease, our investigations and those of others point to critical inflammatory mechanisms causing ARDS in severe COVID-19 patients. Common symptoms of severe COVID-19 disease include: 1) rapid deterioration of disease after one to two weeks; 2) significant decline in lymphocytes, especially natural killer (NK) cells in the blood; 3) elevated pro-inflammatory cytokines (IL-6, TNFα, IL-8, and others) and other inflammatory factors, such as C reactive protein (CRP); and 4) severe immune impairment marked by atrophy of the spleen and lymph nodes and declining lymphocytes in lymphoid organs. Other sequelae commonly observed in severe COVID-19 patients include infiltration of monocytes and macrophages into lung lesions with minimal lymphocyte infiltration, and a form of vasculitis attended by hypercoagulation and multiple organ damage.

An emergent hyperinflammatory condition has now been identified in a small subset of children infected with COVID-19, presently referred to as Pediatric Inflammatory Multisystem Syndrome (PIMS). This Kawasaki-like disease is emerging amid the COVID-19 pandemic globally, as evinced by initial reports in Italy, and parallel reports of similar cases in New York, New Jersey, Massachusetts, and the United Kingdom. While rare, the effects of PIMS are severe and potentially life-threatening.

Emerging data reveal that PIMS is an aberrant immune response to SARS-COV-2 infection, causing Kawasaki-like disease in genetically predisposed pediatric patients. Common symptoms of COVID-19-associated PIMS include fever, rash, red eyes, dry or cracked mouth, redness in the palms of hands and soles of feet, and swollen glands. About one quarter of children have heart complications. The aberrant immune reaction in COVID-19-associated PIMS results in inflammation and swelling of the blood vessels, sometimes with coronary artery aneurysms.

According to the first observational cohort study in Italy (published May 13, 2020 in the Lancet by Verdoni et al.), 8 of 10 children diagnosed with PIMS between Mar. 17-Apr. 14, 2020 tested positive for COVID-19 (with a strong possibility of false negative SARS-COV-2 testing for the remaining two subjects). The Italian the study compared data for this COVID-19 cohort against data for 19 children diagnosed with Kawasaki disease pre-pandemic, over the previous 5 years. Prior to the pandemic, on average there was 1 Kawasaki patient identified every 3 months in the same jurisdictional population, indicating a 30-fold increase in PIMS for the study period during the COVID0-19 outbreak. Children in the Italian study with PIMS diagnosed during the COVID-19 pandemic presented with more severe symptoms than those treated for classic Kawasaki disease over the previous 5 years. 6 of the 10 new patients (60%) exhibited heart complications, compared to only 2 of 19 (11%) among the Kawasaki subjects treated before. 5 of the 10 children in the Italian study (50%) diagnosed during the COVID-19 pandemic exhibited symptoms of toxic shock syndrome (TSS), requiring fluid resuscitation to correct low blood pressure, whereas none of the 19 Kawasaki subjects diagnosed before the COVID-19 outbreak suffered this complication. TSS is a rare, life-threatening illness ordinarily caused by bacterial infections, characterized by high fever, low blood pressure, and rash.

Treatment of PIMS patients in the Italian study involved immunoglobulin therapy, with 8 of the 10 patients also requiring steroid treatment (compared to only 3 of the 19 pre-COVID-19 Kawasaki study group). The children hospitalized during the pandemic in Italy were also older than those diagnosed previously (mean age, 7.5 versus 3 years). Seven of the 10 children were boys.

The US Centers for Disease Control and Prevention (CDC) has now confirmed the link between PIMS and COVID-19. New York City's health department has identified 145 cases of children with PIMS (alternatively referred to as multi-system inflammatory syndrome in children (MISC) to date.

Understanding the mechanisms involved in PIMS will help elucidate the human SARS-CoV-2 immune response more generally, for both adults and children, to guide investigation and development of anti-inflammatory treatment tools and methods useful to alleviate diverse COVID-19 disease symptoms in both groups.

The excessive activity of pro-inflammatory cytokines elicited in severe cases of COVID-19 disease has been popularly referred to as a “cytokine storm”. The term “cytokine storm syndrome” (CSS) (also known as “cytokine release syndrome” or CRS) generally indicates an excessive, uncontrolled release of pro-inflammatory cytokines and other inflammatory factors, leading to excessive and potentially life-threatening inflammation. CSS is correlated with a variety of infectious diseases, rheumatic diseases, autoimmune disorders and in some subjects undergoing tumor immunotherapy.

In the case of infectious disease, CSS usually originates from the focal infected area, but can rapidly spread throughout body. COVID-19 and other SARS coronaviruses (SARS-CoV and MERS) manifest in severe cases with rapid virus replication, extensive inflammatory cell infiltration and CSS leading to ARDS/SARS (acute pulmonary inflammation and injury, culminating in the most severe cases with pulmonary failure and death). CSS associated with COVID-19 disease can also result in systemic inflammation, vasculitis, multiple organ failure, and hypercoagulation potentially leading to stroke in severe cases.

Severe COVID-19 patients exhibit elevated pro-inflammatory cytokine profiles resembling CSS symptoms previously observed in SARS-COV and MERS subjects. Huang et al. (2020) reported that a diverse array of pro-inflammatory cytokines and other inflammatory factors are elevated in patients with serious COVID-19 disease. Among 41 COVID-19 inpatients (13 ICU and 28 non ICU), Huang and colleagues reported increased levels of interleukin (IL)-1B, IL-1RA, IL-7, IL-8, IL-9, IL-10, fibroblast growth factor (FGF), granulocyte-macrophage colony stimulating factor (GM-CSF), IFNγ, granulocyte-colony stimulating factor (G-CSF), interferon-γ-inducible protein (IP10), monocyte chemoattractant protein (MCP1), macrophage inflammatory protein 1 alpha (MIP1A), platelet derived growth factor (PDGF), tumor necrosis factor (TNFα), and vascular endothelial growth factor (VEGF). In the more severe, ICU patients, Huang and coworkers reported that IL-2, IL-7, IL-10, G-CSF, IP10, MCP1, MIP1A, TNFα were higher than in the non-ICU patients (Huang et al., 2020; Conti et al., 2020]. Notably, there was no difference reported for serum IL-6 levels been the ICU and non-ICU patients in the Huang et al. study. Pro-inflammatory cytokines such as interleukin-1 (IL-1) are important mediators in local and systemic inflammation. When stimulated by viral infection IL-1 plays a fundamental role in tissue inflammation, fever and fibrosis. IL-1 activates macrophages which perform phagocytic activity on infected and dead cells and release other inflammatory factors. As such, cytokine-induced macrophages play a central role in COVID-19 excessive inflammation and attendant pulmonary pathogenesis.

Other pro-inflammatory cytokines are implicated in the clinical progression of SARS-COV and COVID-19 disease mediated by CSS. Among these cytokines, interferon-alpha (IFNα), tumor necrosis factor (TNF) and IL-1 have long been regarded as key players (Ho et al., 2003, Auyeung et al., 2005, Chousterman et al., 2017) 2, 18-19). Other pro-inflammatory cytokines suggested as having possible roles in mediating SARS-associated CSS include IL-8, IL-6 and others.

IL·1 is the most thoroughly studied cytokine with properties relevant to inflammatory diseases, including those caused by viral infection. The controlled synthesis and release of IL-1 occurs after binding of CoV-19 to the Toll Like Receptor (TLR). Activation of this receptor causes a pro-inflammatory “cascade” that begins with synthesis of pro-IL-1, which is then cleaved by caspase-1, followed by inflammasome activation (Chen et al., 2006). High levels of adenosine triphosphate (ATP) are correlated with activation of the P2X7 receptor (a purigenic 2 receptor), which mediates inflammasome activation. Pro-caspase-1 and other factors drive synthesis of IL-1b in the lysosome, and IL-1b is then secreted outside the macrophage, mediating lung inflammation, fever and fibrosis associated with severe respiratory problems in susceptible COVID-19 patients. Immune cells are attracted to the locus of infection by IL-8, a chemokine generated at the inflammatory site, further exacerbating the cytokine “storm” in severe cases.

Inflammation is ordinarily an adaptive response evolved to combat injury and defend against foreign substances and pathogens introduced into the body. In contrast, hyperinflammation in the case of CSS associated with COVID-19 disease and other inflammatory conditions is very harmful if not controlled. The documented association between pro-inflammatory cytokine levels and COVID-19 and SARS-COV viral replication and disease severity has prompted many researchers to theorize that anti-inflammatory cytokines might provide useful therapeutic agents to combat these diseases and conditions. Major “anti-inflammatory cytokines” contemplated in this context include interleukin (IL)-1 receptor antagonist (IL-1Ra), IL-4, IL-10, IL-11, and IL-13. Specific cytokine receptors for IL-1, tumor necrosis factor-alpha, and IL-18 are also characterized as “anti-inflammatory” factors, functioning as inhibitors of pro-inflammatory cytokines. Thus, IL-1Ra, IL-37 or IL-13 have been proposed as candidates to alleviate both pulmonary and systemic inflammation and fever in severe COVID-19 disease.

The high case-fatality rate, complex etiology and epidemiology, and the lack of vaccine or therapeutic tools against coronaviruses have created an urgent need for effective prophylactic and therapeutic tools to prevent and treat infection and disease mediated by these pathogens. the vaccine and related therapeutic agents.

Efforts to develop useful drugs and methods to prevent or treat COVID-19 disease have been frustrated in part by the complexity of the SARS viral genome and its confounding disease etiology. SARS-COV, MERS-COV, and SARS-COV-2 have large single-stranded, positive-sense RNA genomes (27-32 kb) encoding 6-10 genes. The gene order s is usually highly conserved, with the first being replication- and transcription-related, and the remaining genes structural. The replication- and transcription-related gene is translated into two large non-structural polyproteins by overlapping open reading frames (ORFs) translated by ribosomal frameshifting. The structural proteins including spike(S), envelope (E), and membrane (M) proteins making up the viral coat, and the nucleocapsid (N) protein that packages the viral genome, all translated from the subgenomic RNAs. Some of these proteins undergo glycosylation in the host Golgi apparatus to form glycoproteins.

The SARS coronavirus spike(S) glycoprotein mediates binding of the virus to host cells to permit intracellular colonization. S protein is primed by the host cell protease and recognized by cellular receptors. The human serine protease TMPRSS2 is responsible for priming the S protein of both SARS-COV and SARS-COV-2, and the angiotensin-converting enzyme 2 (ACE2) is engaged as a receptor for the entry of both viruses. As for MERS-COV, it binds specifically to another receptor, dipeptidyl peptidase 4 (DPP4).

The varied etiology and epidemiology of SARS coronaviruses relates in part to differences between individuals and populations in terms of ACE2 expression levels, an possibly also ACE-2 structural differences between individuals. Children and younger individuals generally express lower levels of ACE-2, which may contribute to their relative resistance to severe COVID-19 disease. Several ACE-2 genetic variants have been identified in human populations, that may further impact viral infection and pathogenicity, however no heterogeneity has been found among ACE-2 residues implicated in the SARS-COV-2 viral S protein binding, indicating the virus likely exploits a highly-conserved attachment/entry site, which correlates with the rapid spread of SARS-COV-2 across continents and different human populations (Cao et al., 2020). In view of these reports, ACE-2 has been widely contemplated as a potential target for intervention in developing anti-COVID-19 prevention and treatment tools, though there remain major obstacles to success along this discovery path.

Another challenging path for innovation toward COVID-19 management relates to the use of anti-inflammatory cytokines and related inhibitors and immune modulators to treat viral-mediated ARDS and CSS in COVID-19 patients. These objectives are complicated by concerns about inhibiting beneficial anti-viral immune and inflammatory functions. Numerous targets have been contemplated for cytokine modulation as a means to combat SARS-COV infection and pathogenesis. Very recent studies have confirmed that severe COVID-19 patients exhibit high erythematosus sedimentation rates (ESR), and persistently high levels of C-reactive protein (CRP), IL-6, TNFα, IL-1B, IL-8, IL2R (Tu et al.). These and other pro-inflammatory markers are clinically associated with ARDS, hypercoagulation and Disseminated Intravascular Coagulation (DIC) (manifesting as thrombosis, thrombocytopenia, and gangrene of extremities in severe cases). These and other sequelae of CSS exacerbate lung damage and can cause systemic CSS-related illness referred to as “extrapulmonary systemic hyperinflammation syndrome” (ESHS), which can cause fetal complications in pregnant subjects.

The question of when and how to block CSS to mediate anti-inflammation therapy is critical for reducing death rates of COVID-19 subjects. In this regard, it is important to consider underlying immune impairment mediated by SARS coronaviruses.

Lymphocytopenia is one of the most prominent diagnostic markers for COVID-19. Both T cells and NK cells in patients with COVID-19 are substantially reduced, while leukocyte counts are elevated. In critically ill patients, NK cells are extremely low, even undetectable, and memory helper T cells and regulatory T cells are profoundly decreased (Hui et al., 2019). Striking COVID-19 autopsy findings also reveal that secondary lymphoid tissues are also destroyed in severe cases—a marked distinction from typical CSS disease. Spleen atrophy is commonly observed, correlated with decreased lymphocytes, significant cell degeneration, focal hemorrhagic necrosis, macrophage proliferation and macrophage phagocytosis in the spleen. Similarly, lymph node atrophy and reduced numbers of lymph nodes are observed, and decreased numbers of CD4+ T cells and CD8+ T cells in the spleen and lymph nodes [Hui et al., 2019]. Additionally, in the lung with characteristic diffused alveolar damage (DAD), the major infiltrated cells were monocytes and macrophages, moderate multinucleated giant cells, with very few lymphocytes. Most of the infiltrating lymphocytes were CD4+T cells. Importantly, virus inclusion bodies are still detected in type II alveolar epithelia and macrophages, even when PCR tests are negative in blood or throat swabs (Zu et al., 2020; De Wit et al., 2016; Chan et al., 2015B). This finding is consistent with a so called “primary cytokine storm” induced by viral infection, mainly elicited by alveolar macrophages, epithelial cells and endothelial cells, rather than a “secondary cytokine storm” induced by different subsets of activated T lymphocytes in late stages of viral infection or complications of T cell-engaging therapies (Chan et al., 2012; Kanne et al., 2020).

Yet another complication of severe COVID-19 disease, generally correlated with ARDS severity, is the phenomenon of neutrophil infiltration into pulmonary capillaries, coupled with formation of neutrophil extracellular traps (NETs), as identified in several recently published COVID-19 autopsies. Neutrophils are the most common type of white blood cell (WBC) in the bloodstream, and are phagocytes which migrate from the blood during the acute phase of inflammation to sites of injury or infection. Neutrophils freely move by chemotaxis from the outset of an infection into and through blood vessels and interstitial compartments, attracted by cytokines (such as Interleukin-8 (IL-8), C5a, fMLP, Leukotriene B4, and H2O2) expressed by activated endothelium, mast cells, and macrophages. Once localized, neutrophils themselves express and release cytokines, which serve to amplify inflammatory reactions through recruitment and activation of other cell types. Neutrophils considerably outnumber monocyte/macrophage phagocytes, and are generally regarded as the hallmark of early, acute inflammation.

In addition to recruiting and activating other cells of the immune system, neutrophils play a key role in front-line defense against pathogens. Neutrophils have three methods for directly attacking viral and bacterial pathogens: phagocytosis (ingestion), degranulation (release of soluble anti-microbials), and generation of neutrophil extracellular traps (NETs). It appears increasingly likely that, in cases of ARDS generally, and SARS-COV infection more specifically, at least some of these normally beneficial immune/inflammatory functions of neutrophils are destructively miscued or overactivated.

Degranulation of neutrophils ordinarily releases an assortment of proteins from three distinct types of granules. Azurophilic granules (or “primary granules”) release myeloperoxidase, bactericidal/permeability-increasing protein (BPI), defensins, and the serine proteases neutrophil elastase and cathepsin G. Specific granules (or “secondary granules”) release alkaline phosphatase, lysozyme, NADPH oxidase, collagenase, lactoferrin, histaminase, and cathelicidin. Tertiary granules release cathepsin, gelatinase, and collagenase. When aberrantly overexpressed or overactivated, these various enzymes and antimicrobial agents can have serious deleterious effects on tissues and extracellular components, including through destruction of essential “barrier” components present in blood vessels, lungs and kidneys that preserve their structural integrity, pathogenic resistance and function.

A more significant pathogenic result of excessive neutrophil numbers, infiltration and activation in COVID-19 disease, appears to involve the generation of neutrophil extracellular traps (NETs). NETs are webs of chromatin, microbicidal proteins, oxidant enzymes and cytokines ordinarily released by neutrophils to contain infections. However, when not properly regulated, NETs appear to propagate inflammation and cause microvascular thromboses, likely contributing to lung failure/ARDS and playing a similar role in vasculitis and secondary organ failure in COVID-19 patients exhibiting CSS and PMIS. A variety of other diseases known to be caused by NETs present with similar symptoms, including thick mucus secretions in the airways and development of blood clots, as seen in ARDS/SARS.

Elevated levels of blood neutrophils predict worse outcomes in COVID-19, and the role of NETs appears most significant. In a recent study, Zuo and coworkers (2020) reported that increased infiltration of neutrophils into capillaries of the lungs and overexpression of NETs correlated strongly with severity of viral pneumonia/ARDS in COVID-19 patients. Sera from patients with severe COVID-19 exhibit elevated levels of cell-free DNA, myeloperoxidase (MPO)-DNA, and citrullinated histone H3 (Cit-H3), indicative of elevated NET levels. Cell-free DNA levels also correlated strongly with acute inflammatory phase reactants, including C-reactive protein, D-dimer, and lactate dehydrogenase. MPO-DNA associated with both cell-free DNA and absolute neutrophil count, while Cit-H3 correlated with platelet levels and observed prothrombic (blood clot forming) effects of NETs. Importantly, both cell-free DNA and MPO-DNA were higher in hospitalized patients receiving mechanical ventilation than those capable of breathing unassisted. Finally, sera from individuals with COVID-19 triggered NET release from control neutrophils in vitro. These data reveal that high levels of neutrophils and NETs in patients with severe COVID-19 likely contribute significantly to CSS, ARDS, vasculitis and thrombus formation in these patients. Other research clearly connects elevation of NETs with ARDS more generally, comparable to these findings for severe SARS-COV infection.

It is important to note that neutrophils may operate beneficially during one period of viral infection or disease, then destructively during another period when miscued or overactivated. In the disease of alpha 1-antitrypsin deficiency, the important neutrophil enzyme elastase, which is ordinarily beneficial for controlling pathogenesis, is not adequately inhibited by alpha 1-antitrypsin and thus causes excessive tissue damage during a normally adaptive inflammatory response (as occurs in pulmonary emphysema).

Excessive activation of neutrophils in other contexts, evidently including COVID-19 disease, releases elastase and other destructive enzymes into extracellular compartments, contributing to pulmonary barrier disruption and acute lung injury. This duality of regulated and unregulated function is reflective of an adaptive process gone wrong, in all likelihood indicating the process is being hijacked or miscued by the subject pathogen, or by virtue of the novel, changing etiology of progressive disease processes.

There are many examples of host-targeted molecular strategies implemented by viruses and bacteria to disable, misdirect or even appropriate host immune and inflammatory processes to benefit the pathogen. In the important case of neutrophils, molecular interactions between these key effector cells and their targeted pathogens can profoundly alter neutrophil proliferation and longevity. Both viral and bacterial pathogens have been proven capable of prolonging neutrophil lifespan, or accelerating neutrophil lysis after phagocytosis. Chlamydia pneumoniae and Neisseria gonorrhoeae have both been shown to inhibit neutrophil apoptosis. Other pathogens are known to extend neutrophil lifespan both by disrupting spontaneous apoptosis, and by inhibiting phagocytosis-induced cell death (PICD). On the other end of the spectrum, some pathogens directly alter neutrophil fate after phagocytosis by accelerating cell lysis and/or apoptosis (a type of neutrophil “overactivation” that causes tissue necrosis).

Viruses and bacteria may also directly attack and destroy elements of the host immune system or inflammatory control machinery. As noted above, severe COVID-19 disease is marked by profound reductions in numbers of lymphocytes, and it is entirely possible that NK cells and other crucial lymphocytes lost in this disease may be directly invaded and destroyed by the SARS-COV virus. On the other hand, the destruction of these cells may be indirect, as an attendant sequel of CSS. Since the COVID-19 virus appears to principally infect target cells via angiotensin converting enzyme 2 (ACE2), and ACE2 expression is absent on lymphocytes, these particular cells are most likely falling victim to CSS destructive mechanisms.

Related to these mechanisms, vasculitis and thrombosis, coupled with endothelial damage, are prominent features in severe COVID-19 patients. Many critical ill COVID-19 patients have vasculitis-like manifestations, often with gangrene at the extremities. Autopsies reveal that pulmonary blood vessels (associated with the alveolar septae) are congested and edematous, with infiltration of monocytes and their macrophage progeny within and around the blood vessels. Small vessels show hyperplasia, vessel wall thickening, lumen stenosis, occlusion and focal hemorrhage. Hyaline thrombi of microvessels are found in severe cases (Zu et al., 2020; Hui et al., 2019; Chan et al., 2015B). The underlying mechanisms of vascular damage may involve direct injury of endothelial cells by virus, or downstream CSS impacts, leading to DIC, anti-phospholipid syndrome (APS) and mimicry of vasculitis. Pathological “autoimmune” responses elicited by anti-virus immunity may also be involved.

COVID-19 patients exhibiting a hypercoagulation state include PIMS subjects. Common sequelae of these complications include prolonged prothrombin time, elevated levels of D-dimer and fibrinogen, and near normal activated partial thromboplastin time. A few patients progress to overt Disseminated Intravascular Coagulation (DIC). Tang et al. (2020) report that 71.4% of non-survivors and 0.6% of survivors of COVID-19 showed evidence of overt DIC. Even more non-surviving patients exhibited latent DIC characterized by a hypercoagulable state in post-mortem examinations (demonstrated by fibrin thrombus formation). A high proportion of acro-ischemia was also observed in deteriorating patients with COVID-19, indicating a hypercoagulable status before the onset of overt DIC. Several factors may contribute to coagulation disorders in COVID-19 patients. The persistent inflammatory status in severe and critical COVID-19 patients acts as an important trigger for the coagulation cascade. Certain cytokines, including IL-6, can activate the coagulation system and suppress the fibrinolytic system. In the setting of COVID-19, pulmonary and peripheral endothelial injury due to direct viral attack may be a key inducer of hypercoagulation. Endothelial cell injury strongly activates the coagulation system, via exposure of tissue factor and other pathways. Aggressive immune and inflammatory responses may in turn be exacerbated by dysfunctional coagulation, with the two processes acting in a sort of feedback loop toward an uncontrolled endpoint. Finally, emergence of antiphospholipid antibodies in COVID-19 patients may intensify their coagulopathy, as anti-cardiolipin and anti-β2GP1 antibodies have been detected in COVID-19 patients Zhang et al., 2020).

While antiviral and supportive treatments are critical components of COVID-19 disease management, blockade of CSS using anti-inflammation therapy represents a critical, unfulfilled objective for treatment and management of COVID-19 disease. A variety of anti-inflammatory medications are well known in the art that might be useful for COVID-19 treatment, for example non-steroidal anti-inflammatory drugs (NSAIDs), glucocorticoids, and chloroquine/hydroxychloroquine, among others. Also broadly contemplated are immunosuppressants, pro-inflammatory cytokine antagonists (such as IL-6R monoclonal antibodies, TNF inhibitors, IL-1 antagonists, janus kinase (JAK) inhibitors, etc.) and other immunomodulatory agents to reduce pulmonary and systemic inflammation.

Despite this large armamentarium of available anti-inflammatory agents, none of these are without uncertainties regarding their efficacy and potential for adverse side effects in the contexts of ARDS and COVID-19 disease. Anti-inflammatory therapy for COVID-19 disease patients presents fundamental risk/benefit concerns, in terms of whether and when to treat subjects with an anti-inflammatory regimen. These fundamental questions remain under intense debate, with no consensus in sight. A principal concern is that anti-inflammatory medications, such as corticosteroids, may delay or impair beneficial anti-viral defenses, and/or concurrently increase risk of secondary infection, particularly in subjects facing pre-existing immune system impairment, or impairment mediated by the virus itself. Other questions arise in the case of biological agents targeting pro-inflammatory cytokines, which may only inhibit one specific inflammatory factor, and thereby fail to curb CSS generally. Alternatively, as with steroids, anti-inflammatory drugs may impair beneficial immune functions. For example, JAK inhibitors may exert potent anti-inflammatory effects, while at the same time impairing crucial immune mechanisms mediated by INF-a. Yet another fundamental confounding question relates to the optimal time window for anti-inflammatory treatment, which appears critical in the case of COVID-19 disease. Severe patients have typically shown a long period of initial, moderate symptoms, followed by abrupt deterioration 1-2 weeks after onset, after which time anti-inflammatory therapy may be unable to achieve a favorable treatment response.

Examining SARS viral etiology in more detail, SARS-COV-2 shows a tropism for, and actively replicates in, the upper respiratory tissues. Like SARS-COV, SARS-COV-2 uses angiotensin-converting enzyme 2 (ACE2) as its main receptor for cellular entry, which is broadly expressed in vascular endothelium, respiratory epithelium, alveolar monocytes, and macrophages (Lu et al., 2020). The main transmission route is through direct or indirect respiratory tract exposure (as demonstrated by live virus isolation from throat swabs, and detection of viral subgenomic messenger RNA (sgRNA) in upper respiratory tract cells) (Wolfel et al., 2020). Tropism for the upper respiratory tissue facilitates the higher contagion of SARS-COV-2, via continuous pharyngeal shedding of the virus, even when symptoms are minimal and restricted to the upper respiratory tract. Later in the disease course, SARS-COV-2 resembles SARS-COV in terms of viral replication advancing to the lower respiratory tract, followed by extensive attack in severe cases against the lungs and other target organs that express ACE2 (including heart, kidney, gastrointestinal tract and distal vasculature). This extent and duration of viral spreading correlates with clinical deterioration, mainly occurring in the second week following disease onset. However, disease exaggeration through the late stage in severe cases is not solely attributable to direct viral damage, but additionally involves immune-mediated injury induced by SARS-COV-2. The two distinctive features of severe and critical patients during this stage of COVID-19 disease are progressive increase of inflammation, and an unusual trend of hypercoagulation. There is no doubt that immune-mediated inflammation plays an important role in this latter stage of severe COVID-19 pathogenesis, as was true in SARS-COV cases. The progression of COVID-19 involves a continuous decrease in lymphocyte count, with significant elevation, infiltration and hyperactivation of neutrophils, coupled with a broad elevation of inflammatory markers (including C-reactive protein, ferritin, interleukin (IL)-6, IP-10, MCP1, MIP1A, and TNFα). Reduced lymphocyte count and elevated levels of ferritin, IL-6 and D-dimer were reported in various studies to be directly associated with increased mortality of COVID-19 patients. Mechanisms underlying the progressive lymphopenia in severe and critical COVID-19 patients remain unclear, though decreases in B cells, T cells, and natural killer (NK) cells are all more prominent in severe cases. Other studies have reported increased levels of CD8+ T-cell activation (measured by proportions of CD38 and HLA-DR expression) despite a reduction in CD8+ T-cell count. Lymphopenia was also an important feature of SARS-COV disease progression, and decline of both CD4+ and CD8+ T lymphocytes often correlated with pathogenic radiographic changes. Although direct infection of macrophages and lymphocytes by SARS-COV was indicated by one study, rapid reduction of lymphocyte counts in SARS-COV-1 was further attributed to two mechanisms, redistribution of circulating lymphocytes or depletion of lymphocytes through apoptosis or pyroptosis. No viral gene expression has been observed in peripheral blood mononuclear cells (PBMCs) of patients with COVID-19, and the normal viral transporter, ACE2, is not expressed on lymphocytes. However, Wang et al. (2020) suggest that T lymphocytes may be more permissive to SARS-COV-2 than to SARS-COV through endocytosis triggered by the spike protein. Other reports indicate there is upregulation of apoptosis, autophagy, and p53 pathways in PBMCs of COVID-19 patients (Xiong et al., 2020), while others report that NK and CD8+ T cells are functionally exhausted in COVID-19 patients through overexpression of NKG2A (Zheng et al., 2020).

The collective findings above indicate that immune disturbance starts early in COVID-19 disease, as a combined result of direct and bystander effects. The progressive pathogenic changes of COVID-19 may be reversible through timely intervention, particularly in mild and moderate cases, however the complexity and refractory nature of this disease presents many obstacles. The clinical course of SARS-COV-2 infection may be conceptually divided into three phases: viremia phase, acute phase (pneumonia phase) and severe or recovery phase. Patients with competent immune functions, without serious risk factors (old age, major co-morbidities, etc.) may generate effective and adequate immune responses to suppress the virus in the first or second phase, without manifesting hyperimmune/hyper-inflammation symptoms of CSS, ARDS or PIMS. In contrast, patients with immune dysfunction may have a higher risk of failing the initial phase and becoming the severe or critical type, with higher morbidity and mortality. Consequently, treatment of COVID-19 should include meticulous triaging and staging of patients, to effectuate treatment for high-risk subjects between the first and the second phases, potentially guided by observations of clinical deterioration (e.g., by detecting abrupt hyperinflammation, threshold levels of lymphopenia, hypercoagulation status, etc.)

In view of the foregoing, there are critical needs in the art for more effective tools and methods to combat Acute Respiratory Distress Syndrome (ARDS), including Severe Acute Respiratory Syndrome (SARS) mediated by coronaviruses, Cytokine Storm Syndrome (CSS) and other hyperinflammatory diseases, including Pediatric Inflammatory Multisystem Syndrome (PIMS), Kawasaki disease, Extrapulmonary Systemic Hyperinflammation Syndrome (ESHS), and vascular congestive and thrombotic conditions associated therewith, including Disseminated Intravascular Coagulation (DIC), thrombosis, stroke, thrombocytopenia, and gangrene, and a wide range of cellular, tissue and organ injuries that attend these conditions.

Related needs remain critically unmet for combatting the profound destruction mediated by COVID-19 and related SARS coronaviruses, which have emerged as novel pathogens three times in the past two decades, and will likely emerge again. Unfulfilled objectives for treating and managing COVID-19 disease are numerous and complex, spanning an enormous range of pathogenic, immunologic and inflammatory insults mediated by COVID-19, that include ARDS/SARS, CSS, PIMS, ESHS, DIC in severe cases.

To address these needs and overcome attendant obstacles will require discovery and development of potent and innovative tools, including multi-activity and multi-targeting drugs and therapies, for example drugs capable of impairing viral functions and regulating hyperimmune and hyperinflammatory responses, while enhancing, or at a minimum not impairing, beneficial host immune and inflammatory capabilities and responses.

Additionally, these objectives will be most profitably met by developing novel combinations of complementary drugs and treatment methods that similarly reach multiple targets and elicit multiple (e.g., antiviral, pro-immune and anti-inflammatory) effects. Successful approaches to combatting COVID-19 and its diverse pathogenic sequelae will be further optimized for individual patients by timing and metering treatment forms, dosages and modalities relative to discrete indicia of disease etiology presented on a patient-specific basis.

### Summary of Exemplary Embodiments of the Invention

The invention achieves the foregoing objects and satisfies additional objects and advantages through the novel and potent use of “TPA” compounds and related compositions and methods to treat and/or prevent an Acute Respiratory Distress Syndrome (ARDS) in a mammalian subject, including Severe Acute Respiratory Syndrome (SARS) in humans mediated by COVID-19 and other SARS coronaviruses. Related aspects of the invention employ clinically effective TPA compounds, compositions and methods to prevent and/or treat hyperimmune and hyperinflammatory conditions, including “Cytokine Storm Syndrome” (CSS) generally, and a host of other hyperinflammatory diseases, including but not limited to Pediatric Inflammatory Multisystem Syndrome (PIMS) associated with COVID-19 disease, the related childhood condition known as Kawasaki disease, Extrapulmonary Systemic Hyperinflammation Syndrome (ESHS), and vascular congestive and thrombotic conditions caused by hyperinflammation, such as Disseminated Intravascular Coagulation (DIC), thrombosis, stroke, thrombocytopenia, and gangrene. Additional aspects of the invention employ TPA compounds, compositions and methods to effectively limit or treat a wide range of cellular, tissue and organ injuries that attend ARDS, SARS, CSS, PIMS, ESHS, DIC and other immune and inflammatory injuries or disease states that can attend these conditions.

Further aspects of the invention are directed to compositions and methods employing a TPA compound to treat and/or prevent COVID-19 disease and related adverse conditions and symptoms in humans caused by a SARS coronavirus (e.g., SARS-COV, SARS-COV-2 (COVID-19) and Middle East Respiratory Syndrome coronavirus (MERS-COV)). In exemplary embodiments, the invention provides clinically effective TPA compounds, compositions and methods that prevent and/or treat ARDS/SARS in severe COVID-19 disease subjects. In other exemplary embodiments, compositions and method of the invention employ a TPA compound or composition to effectively treat and/or prevent hyperimmune and hyperinflammatory conditions, and associated cellular, tissue and organ injury and dysfunction, attending severe COVID-19 disease, including CSS, PIMS, ESHS, DIC and other immune and inflammatory injuries or disease states that may attend these conditions, such as vascular congestive and thrombotic conditions, including but not limited to Disseminated Intravascular Coagulation (DIC), thrombosis, stroke, thrombocytopenia, and gangrene.

Effective TPA compounds within the methods and compositions of the invention include phorbol esters and derivatives according to Formula I, below:

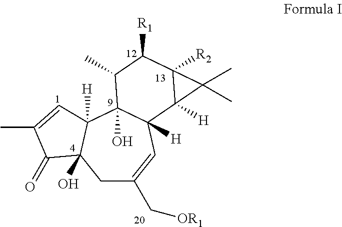

wherein R₁ and R₂ may be hydrogen; hydroxyl;

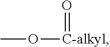

wherein the alkyl group contains 1 to 15 carbon atoms;

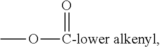

wherein a lower alkenyl group contains between 1 to 7 carbon atoms;

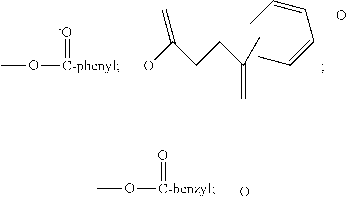

and
wherein R₃ may be hydrogen or

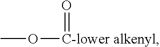

In some embodiments, at least one of R₁ and R₂ are other than hydrogen and R₃ is hydrogen or

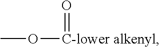

and substituted derivatives thereof. In other embodiments, either R₁ or R₂ is

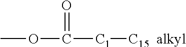

and the remaining R₁ or R₂ is a

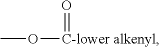

wherein a lower alkyl is between 1 and 7 carbons, and R₃ is hydrogen.

The alkyl, alkenyl, phenyl and benzyl groups of the formulas herein may be unsubstituted or substituted with halogens, preferably, chlorine, fluorine or bromine, nitro, amino, and/or similar type radicals.

In other embodiments, the invention employs exemplary phorbol esters, of Formula II, below:

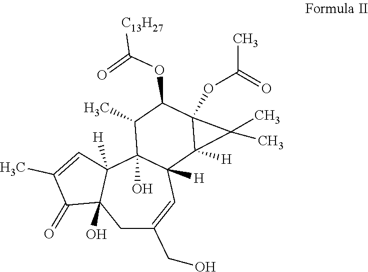

Phorbol esters for use within the compositions and methods of the invention will be understood to include pharmaceutically acceptable active analogs and derivatives of the Formula I and Formula II compounds, including rational designed chemical analogs and derivatives with selected substitutions, deletions or additions of functional groups applied to a parent compound structure within Formula I or Formula II, as well as salts, isomers, enantiomers, polymorphs, solvates, hydrates, and/or prodrugs of said compounds.

### DETAILED DESCRIPTION OF EXEMPLARY EMBODIMENTS OF THE INVENTION

The following description is provided to illustrate fundamental means and principles of the invention, through explanation of exemplary embodiments. No limitation of the invention is intended by this description. Persons of ordinary skill in the art will appreciate that alterations, modifications, substitutions, refinements and further applications of the objects, materials and principles described here fall within the inventive scope of this disclosure and the appended claims.

The instant invention provides “TPA compounds” as drug agents, and for use in drug combinations and methods, to mediate novel, diverse and often multi-functional therapeutic effects. The TPA compounds and related compositions and methods of the invention are effective to treat and prevent a diverse array of refractory disease conditions mediated by aberrant, hyperimmune and hyperinflammatory responses in humans and other mammals. TPA compounds, compositions and methods of the invention potently exert one or more effective activities, including antiviral, anti-inflammatory, and immune-regulatory activities, to ameliorate immune dysfunctional and hyperinflammatory diseases and conditions caused by viral and bacterial infection, burns, trauma and other insults that trigger harmful impairment, mis-direction or escalation of normally beneficial immune and inflammatory mechanisms.

Among the novel and potent uses for TPA compounds described here, the invention provides effective clinical tools and methods to prevent and treat a previously intractable condition known as Acute Respiratory Distress Syndrome (ARDS). ARDS is a complex immune dysfunctional and hyperinflammatory disease mediated by complex pathogenic factors and mechanisms. ARDS may be caused by burns or chemical injuries to the lungs, traumatic injuries to the lungs, or overwhelming infection of the lungs by viral or bacterial factors, among other causes. Unifying features of ARDS include profound inflammation of the lung parenchyma, infiltration of excessive numbers of destructive neutrophils and macrophages into the lung tissue and pulmonary alveolar spaces, oxidative stress, disruption of endothelial and epithelial barriers, damage to the pulmonary epithelial lining and lung fibrosis. The impacts of these pathogenic developments on patients lead to restricted, labored breathing, hypoxemia and eventual pulmonary failure, long-term fibrosis and other pathological injury, and death in severe cases. Within related aspects of the invention, TPA methods and compositions are employed for treating a form of ARDS mediated by SARS coronaviruses, including the COVID-19 virus, referred to as Severe Acute Respiratory Syndrome (SARS).

Within other aspects of the invention, effective TPA compounds, compositions and methods are used to prevent and treat hyperinflammatory conditions, including “Cytokine Storm Syndrome” (CSS) generally, Pediatric Inflammatory Multisystem Syndrome (PIMS) associated with COVID-19 disease, Kawasaki disease, Extrapulmonary Systemic Hyperinflammation Syndrome (ESHS) generally, and vascular congestive and thrombotic conditions caused by hyperinflammation, including Disseminated Intravascular Coagulation (DIC), thrombosis, stroke, thrombocytopenia, and gangrene.

Yet additional aspects of the invention employ TPA compounds and methods to directly prevent or inhibit productive infection by a coronavirus, particularly a SARS coronavirus. TPA compounds of the invention are effective in human subjects to treat and prevent SARS-COV-2 infection and disease, as evinced by TPA's ability to elicit anti-SARS-COV-2 immune-regulatory, immune-enhancing, and/or anti-inflammatory responses in COVID-19 disease subjects. The multiple anti-viral activities of TPA compounds described herein are effective to prevent or reduce SARS-COV-2 infection—by reducing viral load in the upper and lower respiratory tract; lowering viral titer in non-respiratory, ACE-2 positive cell and tissues; preventing or reducing viral attachment and entry into lung and other ACE-2 positive cells and tissues; preventing or reducing viral replication in lung and other ACE-2 positive cell and tissues; and/or by preventing or reducing viral shedding from the upper respiratory tract of infected subjects. These anti-viral effects may be direct or indirect, though the anti-viral outcome is specific to prevent or limit viral infection, replication, pathogenicity, transmissibility and related COVID-19 disease severity in treated subjects. More detailed aspects of the invention are directed to compositions and methods employing a TPA compound, composition or method to treat and/or prevent COVID-19 disease and/or related adverse conditions and symptoms caused by a SARS coronavirus (SARS-COV, SARS-COV-2 (COVID-19), or Middle East Respiratory Syndrome coronavirus (MERS-CoV)).

In exemplary embodiments, the invention provides clinically effective TPA compounds that function as “anti-viral” drugs. As used herein, anti-viral activity includes, for example, reducing viral load/titer in a targeted cell, tissue or compartment, or impairing or otherwise limiting a viral function (e.g., viral attachment, viral entry into cells, viral replication, viral shedding, viral defenses against host anti-viral mechanisms, pathogenic impacts of virus on host cell integrity, physiology, gene expression, immune activity, inflammatory activity, life-cycle/longevity, etc.) These and related anti-viral methods and compositions will prevent and/or COVID-19 infection and/or attendant disease symptoms in at-risk, virus-exposed subjects. In other exemplary embodiments, compositions and method of the invention employ a TPA compound or composition to effectively treat and/or prevent hyperimmune and hyperinflammatory conditions, and associated cellular, tissue and organ injury and dysfunction, attending severe COVID-19 disease, including CSS, PIMS, ESHS, DIC and other immune and inflammatory injuries or disease states that may attend these conditions, such as vascular congestive and thrombotic conditions, including but not limited to Disseminated Intravascular Coagulation (DIC), thrombosis, stroke, thrombocytopenia, and gangrene.

In certain embodiments the invention employs one or more TPA or TPA-like compounds, either a parent TPA compound (12-O-tetradecanoylphorbol-13-acetate (TPA); also known as phorbol-12-myristate-13-acetate (PMA)), or a structurally related, functional analog, derivative, salt or other derivative form of this parent TPA compound. TPA compounds employed within the invention are useful in compositions and methods administered to subjects to mediate anti-viral, anti-ARDs, anti-CSS, anti-inflammatory, anti-cytopathic, pro-immune, and apoptosis-regulating effects, among other clinically-relevant activities. These activities are individually or cooperatively effective in discrete disease contexts, to mediate prevention and/or treatment of a range of ARDS and SARS virus (e.g., COVID-19) disease conditions, symptoms and attendant immunological, cellular, tissue and organ injuries and dysfunctions.

TPA compounds for use within the invention include one or more phorbol ester compound(s) of Formula I and/or Formula II, effectively administered to prevent and/or treat a targeted viral infection, disease, condition or symptom as described herein. In exemplary embodiments, a parent or prototype TPA compound, phorbol 12-myristate-13-acetate (“PMA”, alternatively referred to herein as 12-O-tetradecanoyl-phorbol-13-acetate “TPA”)) is utilized as a clinically effective agent in pharmaceutical compositions and methods of the invention, for illustrative purposes. It will be recognized, however, that the instant disclosure provides many additional, pharmaceutically acceptable phorbol ester compounds in the form of a native or synthetic compound, complexes, analogs, derivatives, salts, solvates, isomers, enantiomers, polymorphs, and prodrugs of the compounds according to the structural foundations described for Formula I and Formula II compounds below.

Phorbol is a natural, plant-derived polycyclic alcohol of the tigliane family of diterpenes. It was first isolated in 1934 as the hydrolysis product of croton oil derived from the seeds of Croton tiglium. It is well soluble in most polar organic solvents and in water. Esters of phorbol have the general structure of Formula I, below:

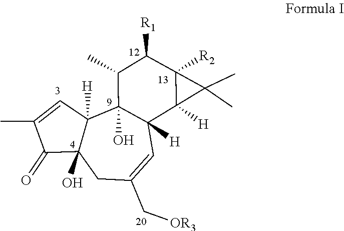

wherein R₁ and R₂ are selected from the group consisting of hydrogen;

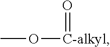

wherein the alkyl group contains 1 to 15 carbon atoms,

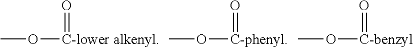

and substituted derivatives thereof and R₃ may be hydrogen,

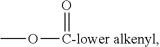

or substituted derivatives thereof.

The term “lower alkyl” or “lower alkenyl” as used herein means moieties containing 1-7 carbon atoms. In the compounds of the Formula I, the alkyl or alkenyl groups may be straight or branched chain. In some embodiments, either or both R₁ or R₂, are a long chain carbon moiety (i.e., Formula I is decanoate or myristate).

The alkyl, alkenyl, phenyl and benzyl groups of the formulas herein may be unsubstituted or substituted, for example with halogens (e.g., chlorine, fluorine or bromine), nitro, amino and other functionalities.

Organic and synthetic forms of phorbol esters, including any preparations or extracts from herbal sources such as Croton tiglium, are contemplated as useful compositions comprising phorbol esters (or phorbol ester analogs, related compounds and/or derivatives) for use within the embodiments herein. Useful phorbol esters and/or related compounds for use within the invention will typically have a structure according to Formula I, although functionally equivalent analogs, complexes, conjugates, and derivatives of such compounds will also be appreciated by those skilled in the art as residing within the scope of the invention.

In more detailed embodiments, illustrative structural modifications according to Formula I above will be selected to provide useful candidate compounds for treating and/or preventing COVID-19 infection or disease, ARDS generally, SARS, CSS, PIMS and other targeted infections, diseases, conditions and symptoms described herein. Among the diverse modifications of Formula I compounds contemplated here, exemplary analogs and derivatives can be routinely constructed and tested wherein: at least one of R₁ and R₂ are other than hydrogen and R₃ is selected from the group consisting of hydrogen

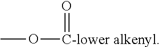

and substituted derivatives thereof. In another embodiment, either R₁ or R₂ is

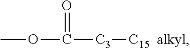

the remaining R₁ or R₂ is

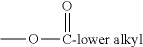

and R₃ is hydrogen.

Alternatively, certain rationally designed analogs and derivatives according to Formula II can be constructed wherein R₁ and R₂ may be hydrogen; hydroxyl;

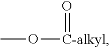

wherein the alkyl group contains 1 to 15 carbon atoms;

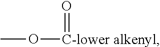

wherein a lower alkenyl group contains between 1 to 7 carbon atoms;

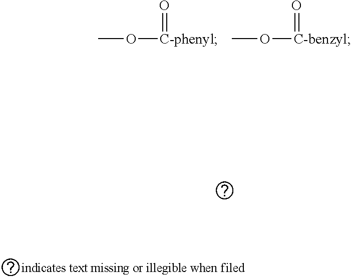

o and substituted derivatives thereof, and wherein R₃ may be hydrogen or

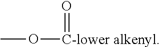

Other illustrative clinical compositions and methods of the invention exemplified here employ phorbol 12-myristate-13-acetate (TPA) according to Formula II, below.

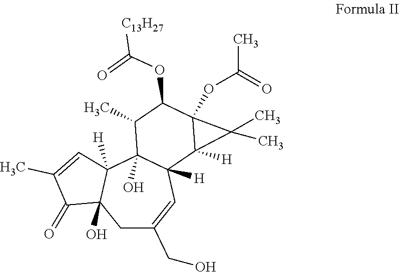

encompassing pharmaceutically acceptable active salts, active isomers, enantiomers, polymorphs, glycosylated derivatives, solvates, hydrates, and/or prodrugs thereof. Additional exemplary phorbol esters for use within the compositions, but are not limited to, phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; and phorbol 13-acetate.

For use in anti-viral, anti-ARDS, anti-CSS and other principal therapeutic compositions and methods of the invention, additional TPA compounds may be made by structural modifications implemented according to rational design chemistry, to improve such relevant pharmacological properties as solubility, lipophilicity, bioavailability, half-life in vivo, resistance or susceptibility to endogenous enzymes, amenability to formulation for specific delivery forms, modalities, and delivery targets/compartments, shelf life stability, etc. Additional useful phorbol esters and related compounds and derivatives within the formulations and methods of the invention include, but are not limited to, other pharmaceutically acceptable active salts of said compounds, as well as active isomers, enantiomers, polymorphs, glycosylated derivatives, solvates, hydrates, and/or prodrugs of said compounds. Further exemplary forms of phorbol esters for use within the compositions and methods of the invention include, but are not limited to, phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12, 13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; and phorbol 13-acetate. Illustrative of these diverse targets for rational design chemical modification of parent phorbol ester compounds are the structures shown in Table 1.

TABLE 1
Exemplary Phorbol Esters

| | |
|---|---|
| Phorbol 13- Butyrate | 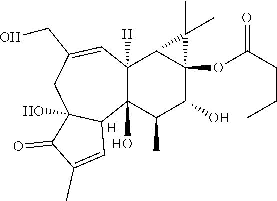 |
| Phorbol 12- | [symbol] |
| Decanoate | |
| Phorbol 12,13- Dibenzoate | 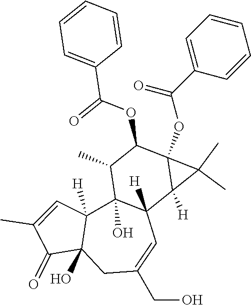 |
| Phorbol 12,13- Dibutyrate | 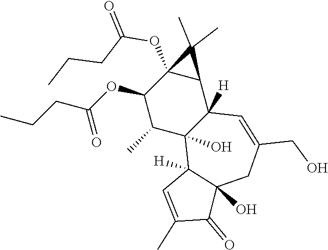 |
| Phorbol 12,13- Didecanoate | 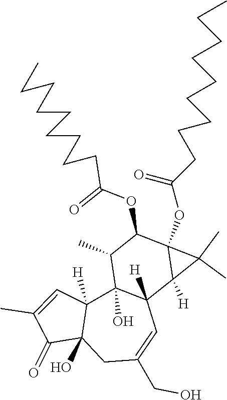 |
| Phorbol 12,13- Dihexanoate | 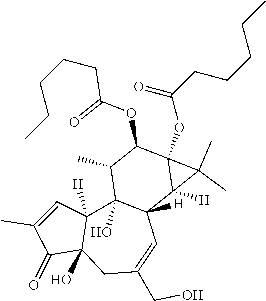 |
| Phorbol 12,13- Dipropionate | 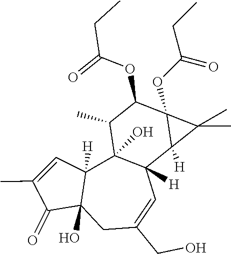 |
| Phorbol 12- Myristate | 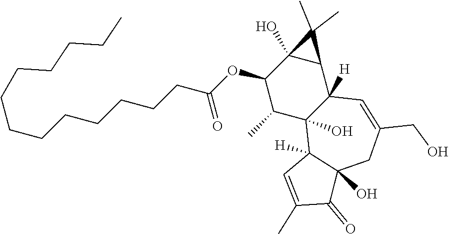 |
| Phorbol 13- Myristate | 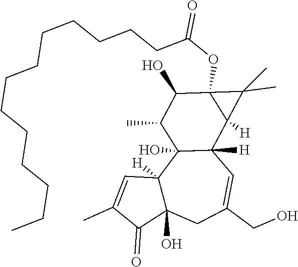 [SMILES: CCCCCCCCCCCCCC(=O)O[C@@]12[C@H](O)[C@@H](C)[C@@]3(O)[C@@H](C=C(CO)C[C@]4(O)C(=O)C(C)=C[C@@H]34)[C@@H]1C2(C)C] |
| Phorbol 12- Myristate-13- Acetate (also known as TPA or PMA) | 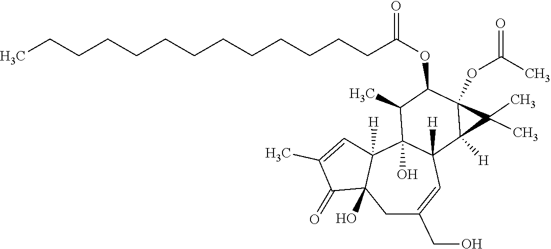 |
| Phorbol 12,13,20- Triacetate | 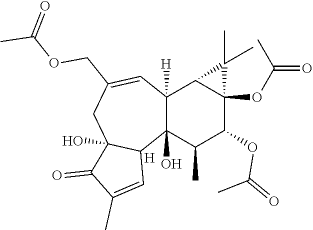 |
| 12-Deoxyphorbol 13-Angelate | 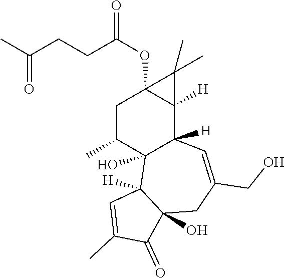 [SMILES: CC(=O)CCC(=O)O[C@@]12C[C@@H](C)[C@@]3(O)[C@@H](C=C(CO)C[C@]4(O)C(=O)C(C)=C[C@@H]34)[C@@H]1C2(C)C] |
| 12-Deoxyphorbol 13-Angelate 20- Acetate | 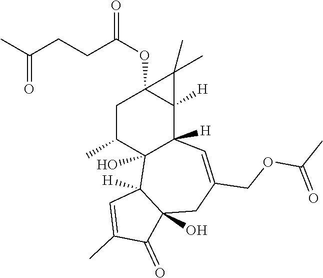 [SMILES: CC(=O)CCC(=O)O[C@@]12C[C@@H](C)[C@@]3(O)[C@@H](C=C(COC(C)=O)C[C@]4(O)C(=O)C(C)=C[C@@H]34)[C@@H]1C2(C)C] |
| 12-Deoxyphorbol 13-Isobutyrate | 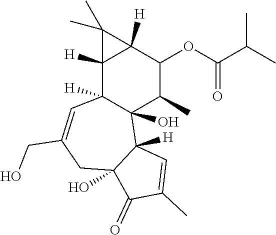 [SMILES: CC1=C[C@H]2[C@@]3(O)[C@H](C)C(OC(=O)C(C)C)[C@@H]4[C@H]([C@@H]3C=C(CO)C[C@]2(O)C1=O)C4(C)C] |
| 12-Deoxyphorbol 13-Isobutyrate-20- Acetate | 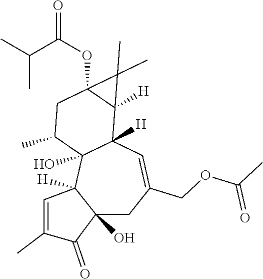 [SMILES: CC(=O)OCC1=C[C@H]2[C@@H]3C(C)(C)[C@]3(OC(=O)C(C)C)C[C@@H](C)[C@]2(O)[C@@H]2C=C(C)C(=O)[C@@]2(O)C1] |
| 12-Deoxyphorbol 13-Phenylacetate | 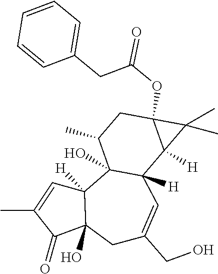 |
| 12-Deoxyphorbol 13-Phenylacetate 20-Acetate | 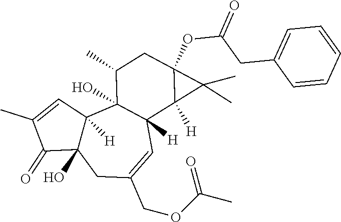 |
| 12-Deoxyphorbol 13- Tetradecanoate | 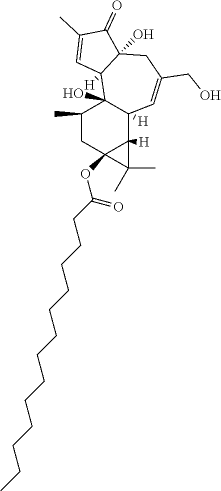 [SMILES: CCCCCCCCCCCCCC(=O)O[C@@]12C[C@@H](C)[C@]3(O)[C@H]4C=C(C)C(=O)[C@@]4(O)CC(CO)=C[C@H]3[C@@H]1C2(C)C] |
| Phorbol 12- Tigliate 13- Decanoate | 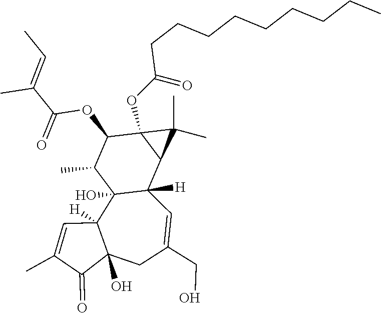 [SMILES: C/C=C(\C)C(=O)O[C@@H]1[C@@H](C)[C@@]2(O)[C@@H](C=C(CO)C[C@]3(O)C(=O)C(C)=C[C@@H]23)[C@@H]2C(C)(C)[C@]12OC(=O)CCCCCCCCC] |
| 12-Deoxyphorbol 13-Acetate | 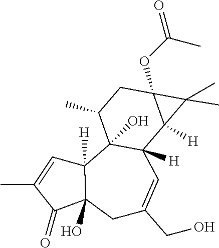 |
| Phorbol 12- Acetate | 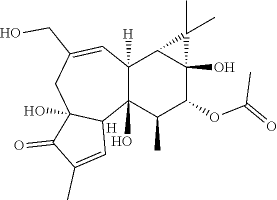 |

[symbol]

indicates data missing or illegible when filed

### Anti-Viral Compositions and Methods

The invention provides novel tools and methods for treatment and prevention of viral infections and related disease, including treatment and prevention of SARS-COV-2 infection and attendant COVID-19 disease. In exemplary embodiments, subjects at risk for becoming infected with SARS-COV-2, and infected subjects testing positive for the virus, are administered an anti-viral effective amount of a TPA compound, sufficient to elicit an antiviral response to SARS-COV-2, and/or to prevent or reduce one or more clinical symptoms of COVID-19 disease. In certain embodiments, the TPA compound is administered in an amount and dosage form that is effective to reduce or eliminate one or more indicia of viral infection severity, selected from: 1) Viral load/titer in an upper or lower respiratory cell, tissue or sample of the subject; 2) viral load/titer in a non-respiratory, ACE-2 positive cell, tissue or sample of the subject, or in a blood plasma of the subject; 3) Viral attachment and/or entry into lung or other tissues/cells; 4) Viral replication in a lung or other ACE-2 positive cell, tissue or organ of the subject; and/or 5) Viral shedding from an upper respiratory tract tissue or sample of infected subjects (wherein each indicator/value is measured and determined in treated subjects, in comparison to the same indicator/value measured and determined in similar, placebo-treated control subjects). Demonstrating anti-viral efficacy of TPA through clinical assessment of treated and control subjects to determine a viral “load” in the subjects quantitively compares a count or estimated “titer” of SARS-COV-2 virions, DNA or other quantitative measure of SARS-COV-2 levels in test and control biological samples (e.g., nasopharyngeal swab samples). A variety of assay methods and materials are published and widely known and routinely implemented for this purpose. According to the teachings herein, patients treated with anti-viral TPA compositions and methods of the invention will show at least a 20% reduction, often a 25-50% reduction, in many cases a 75-95% or greater reduction, up to 100% elimination of detectable viral load/titer, compared to placebo-treated control subjects similarly at risk or infected by SARS-COV-2. Comparable clinical efficacy is likewise provided for other SARS coronaviruses (SARS-COV and MERS), and other viruses and pathogens that cause, or contribute to, ARDS, CSS or PIMS in human subjects.

Related compositions and methods of the invention directed to anti-viral treatment of SARS-CoV-2-positive patients, therapeutic TPA compositions are capable of eliminating or clearing the virus, at least from the upper respiratory tract, within 1-2 weeks following treatment. In particular, DNA or other quantitative measures of SARS-COV-2 levels in nasopharyngeal swab samples from subjects screened as positive for SARS-COV-2 infection before TPA treatment, will be decreased within two weeks after TPA treatment by 100% (corresponding to total clearance of detectable SARS-COV-2 virus in the upper respiratory tract, indicative of a non-contagious status), in at least 25-50%, 50-75%, up to 90% or more of TPA-treated subjects. In related embodiments, patients testing positive for SARS-COV-2, or presenting with known elevated risk factors for COVID-19 disease are carefully monitored (e.g., through blood tests for cytokines, lung function and hypoxemia testing, scans for pulmonary pathogenic lesions, etc.) to ensure that anti-COVID-19 TPA treatment is initiated before (or as soon after as possible), the subject develops one or more index (ices) of severe COVID-19 disease selected from: 1) fever lasting over 2 days; 2) serious lower respiratory symptoms, including pulmonary congestion, tightness, shortness of breath and/or hypoxemia; and/or 3) any symptom of an acute respiratory distress syndrome (ARDS), cytokine storm syndrome (CSS); Pediatric Inflammatory Multisystem Syndrome (PIMS); Extrapulmonary Systemic Hyperinflammation Syndrome (ESHS), and/or any other condition or symptom associated with a hyper-immune hyper-inflammatory response in the subject, including vascular congestive and thrombotic conditions, Disseminated Intravascular Coagulation (DIC), thrombosis, stroke, and/or thrombocytopenia. Without wishing to be bound by theory, TPA compounds of the invention exert multiple anti-viral activities in treated subjects, both direct and indirect, which are effective to mediate both prophylactic (i.e., to prevent or reduce the incidence or extent of actual infection) and therapeutic (to reduce or eliminate viral load and thereby lower disease severity) impacts and responses in treated subjects.

Certain anti-viral effects of TPA compounds involve their pro-immune and anti-inflammatory interactions with protein kinases. Protein kinases are principal components of biological signal-response pathways that “regulate” immune and inflammatory responses to viruses and other pathogens. In one aspect of the invention, interactions between TPA compounds, and the one or more protein kinases involved in immune-regulation and inflammation, mediates potent anti-viral effects to prevent and treat SARS-COV-2 infection.

Protein Kinase C (PKC) is an important kinase for regulating immune functions and inflammation in humans. As used herein, “regulate” means to beneficially control, by activation, suppression, and/or selection of a specific immune or inflammatory signal or trigger (e.g., a cytokine or chemoattractant signal), cell, mechanism or pathway. In the context of PKC, TPA compounds of the invention bind to and activate PKC, which binding and activation in SARS-CoV-2 patients mediates transcriptional activation of interferon-stimulated genes (ISGs) involved in pro-immune signaling and downstream activation of immune effector functions. In exemplary embodiments, TPA-activated PKC stimulates ISGs encoding products capable of interfering with viral replication and spread, including by slowing cell metabolism and activating adaptive immunity. Other ISGs activatable by TPA include pattern recognition receptors (PRRs), which further sensitize cells to pathogens, proteins that decrease membrane fluidity and permeability (inhibiting membrane fusion and impairing viral entry and egress), and other antiviral factors that inhibit specific targets involved in viral cellular entry, replication, and release/shedding cycles (Schneider et al., 2014; Totura et al.)

Human interferons comprise a large group of proteins that help regulate activity of the immune system. Generally, type interferons (IFN-1) provide potent anti-viral effects. In COVID-19 infection, IFN-1 is produced by immune cells, epithelial cells and endothelial cells in early response to viral infection. The possible use of type 1 interferons (IFN-I) for COVID-19 treatment has been surveyed very recently by Sallard et al (2020). Upon recognition of viral components by pattern recognition receptors (PRRs), IFN-1s are among the first immune effector molecules produced during most viral infections. IFN-1s are recognized by the IFNAR receptor present at the plasma membrane in diverse cell types. IFN-1 binding with the IFNAR induces phosphorylation of transcriptional factors such as STAT1 and STAT2 and directs their localization to the nucleus, where they activate interferon-stimulated genes (ISGs).

ISGs encode an array of important molecules responsible for regulating diverse immune responses, including inflammation, cytokine signaling and immunomodulation. Certain ISGs interfere with viral replication and spread, through a variety of mechanisms (Schneider et al., 2014; Totura et al.) At the same time IFN-1 activation stimulates downstream, pro-inflammatory cytokines, which raises some concerns regarding potential of IFN-s to elicit or contribute to Cytokine Storm Syndrome (CSS).

The instant invention regulates IFN-1 activation (through TPA-activation of PKC), yielding safe and discrete anti-viral effects. In particular, the TPA compositions and methods enhances anti-viral protections mediated by IFNs without increasing inflammation or suppressing other beneficial immune functions.

IFN-I treatment has been investigated against MERS-COV and SARS-COV (reviewed in Stockman et al., 2006), alone and in combination with lopinavir/ritonavir (Chan et al., 2015; Sheahan et al., 2020), ribavirin (Chen et al., 2004; Morgenstern et al., 2005; Omrani et al., 2014), remdesivir, corticosteroids (Loutfy et al., 2003), or IFNγ (Sainz et al., 2004; Scagnolari et al., 2004). IFNα and β reportedly showed anti-viral activity in vitro and in certain animal models (Chan et al., 2015), but failed to significantly improve the disease in humans (Stockman et al., 2006). A combination of IFNβ with lopinavir/ritonavir against MERS-COV did improve pulmonary function, but did not significantly reduce virus replication or lung pathogenesis (Sheahan et al., 2020). A combination of IFNα2a with ribavirin delayed but did not significantly reduce eventual mortality (Omrani et al., 2014). A combination of IFNα2b with ribavirin gave positive results in the rhesus macaque (Falzarano et al., 2013), but was inconclusive in humans (Arabi et al., 2017).

The failure to demonstrate substantial disease improvement with IFN-I treatment in these prior studies may be related to viral inhibition of IFN signaling pathways by MERS-COV and SARS-CoV, by the limited number of subjects used in the studies, or by the difficulty of discerning individual effects of IFN-I when used in combination with other drugs (Sallard et al., 2020). It may also be the case that IFN is more effective in patients who lack comorbidities (Al-Tawfiq et al., 2014; Shalhoub et al., 2015). IFN subtype diversity may be another explanation for noted inconsistencies between these studies. It has been repeatedly shown that IFNβ is a more potent inhibitor of coronaviruses than IFNα (Scagnolari et al., 2004; Stockman et al., 2006). This subtype superiority may be related to the protective activity of IFNβ1 in the lungs, where IFNβ1 up-regulates cluster of differentiation 73 (CD73) in pulmonary endothelial cells, promoting secretion of anti-inflammatory adenosine and supporting maintenance of endothelial barrier function. These beneficial effects are correlated with observed reduction of vascular leakage among ARDS patients treated with IFNβ1a (Bellingan et al., 2014), though this effect alone was insufficient to decrease ARDS mortality (Ranieri et al., 2020).

Recent studies suggest that timing of IFN-I administration during SARS coronavirus disease development may play a critical role in determining therapeutic outcome. In one study, positive effects were observed if IFN-I was administered shortly after infection, but IFN-I failed to inhibit viral replication and elicited negative side-effects when administered later (Channappanavar et al., 2019).

Pilot studies by the instant inventors indicate that early administration of TPA will yield clinically beneficial anti-viral effects against SARS-COV-2. Usually TPA treatment will begin as soon as a patient is positively diagnosed and referred for treatment. If diagnosis and referral happen very early, however, it may be desirable to hold treatment until substantial symptoms develop. For example, if a patient is positive for virus in the upper respiratory tract, but has no signs of severe infection (e.g., fever, labored breathing/hypoxemia), then treatment may be delayed until such signs first appear (usually around day 7-8 from onset of symptoms). This delay may be beneficial so as to time the treatment optimally to counter disease escalation while not exhaust immune machinery, particularly lymphocyte reserves, too early.

The use of TPA compounds described herein to stimulate PKC and IFN-1 is particularly promising by virtue that this mechanism should evade SARS viral counter-immune strategies. It is reported that SARS-COV is able to disrupt certain immune signaling pathways in their hosts. For example, the Orf6 protein of SARS-COV disrupts karyopherin transport and consequently inhibits import into the nucleus of transcriptional factors, including STAT1, thus impairing the hosts STAT-1-mediated interferon response. Similarly, the Orf3b protein of SARS-COV inhibits phosphorylation of IRF3, another factor involved in IFN activation (Frieman et al., 2007; Kopecky-Bromberg et al., 2007). However, the Orf6 and Orf3b proteins of SARS-COV-2 are truncated, which may explain why SARS-COV-2 displays substantial sensitivity to IFNα in vitro (Lokugamage et al., 2020). SARS-COV-2 is substantially more sensitive to IFN-I than SARS-CoV, suggesting that IFN-I treatment may be more effective against COVID-19 disease. Supporting this hypothesis, it has been reported that IFNα2b sprays can reduce infection rates of SARS-COV-2 (Shen and Yang, 2020). This report of IFN-I prophylaxis against SARS-COV-2 is consistent with data reported on in vitro efficacy of interferon pretreatment against the SARS-CoV-2 virus (Lokugamage et al., 2020).

Militating in favor of early TPA treatment for SARS-COV-2, we emphasize that the pathology of COVID-19 involves extensive pulmonary lesions in severe cases. Some researchers have compared these lesions to certain “interferonopathies” associated with excessive use of IFN drug therapy (e.g., in multiple sclerosis patients). It is further possible that the SARS-COV-2 virus may induce an excessive IFN response, within the more general “cytokine storm” response, thereby contributing to CSS and pulmonary injury in severe cases (Siddiqi and Mehra, 2020; Zhang et al., 2020). Thus, while it is believed that TPA treatment according to the invention activates IFN-1 and ISGs without excessive (hyperinflammatory) stimulation of cytokines to levels associated with CSS, ARDS, and PIMS, TPA treatment will often be directed to an early stage of SARS-COV-2 infection. Typically, TPA treatment will be initiated before 7-10 days following diagnosis. In certain cases, where ARDS and CSS risks are elevated, or symptoms are observed, TPA treatment may be terminated concurrently or soon thereafter (e.g., at a point when substantially elevated levels of pro-inflammatory cytokines are observed, and/or when substantial ARDS symptoms are observed). In other aspects of the invention, the TPA compound is administered at a selected point during the first stage of a SARS-COV-2 infection (for example 3-7 days after upper respiratory tract symptoms are noted, confirmed by a positive nasopharyngeal SARS-COV-2), then maintained for a period of 1-5 days, then used in combination with an anti-interferon drug 10-12 days after symptom onset (or beginning when substantially elevated levels of pro-inflammatory cytokines are observed, and/or when substantial ARDS symptoms are observed).

The novel TPA compositions and methods of the invention also mediate indirect anti-viral effects through pro-immune activation and rescue of lymphocytes to reduce the critical lymphocytopenia mediated by SARS-COV-2 and other SARS coronavirus. Perhaps the most deleterious impact observed in COVID-19 disease subjects is the precipitous crashing of lymphocytes, including T cells, B cells and NK cells. In the most severe COVID-19 patients, NK cells are extremely low or absent, and memory helper T cells and regulatory T cells are profoundly decreased (Hui et al., 2019). This correlates with autopsy evidence that secondary lymphoid tissues are destroyed and spleen atrophy is observed in fatal COVID-19 cases.

Coupled with these developments, severe COVID-19 patients show severe reduction of lymphocytes, along with evidence of hemorrhagic necrosis, macrophage proliferation and macrophage phagocytosis in the spleen. Similar pathogenic changes are seen in the lungs of severe COVID-19 patients, presenting with ARDS marked by extreme low numbers of lymphocytes, prominent macrophage and neutrophil infiltration, epithelial and endothelial barrier disruption, and diffused alveolar damage (DAD) in the lung parenchyma. This occurs while viral inclusion bodies are still detected in the lung epithelium, even when PCR tests are negative in blood or throat swabs (Zu et al., 2020; De Wit et al., 2016; Chan et al., 2015B). These pathogenic indicia are likewise correlated with elevated cytokine levels, collectively indicating the condition known as “cytokine storm syndrome” (CSS).

In addition to minimizing hyper-inflammatory activities of macrophages and neutrophils, TPA compositions and methods of the invention mediate protection of lymphocytes (prolongation of lifespan), and also directly stimulate lymphocyte proliferation and differentiation. Our pilot studies show a direct, clinically effective mitogenic effect of TPA on peripheral blood lymphocytes (predominantly T-cells). As it appears that SARS-COV-2 overwhelms and exhausts T, B and NK cells, the evidence indicates that TPA compounds and methods will effectively reduce or prevent lymphocytopenia in COVID-19 patient. This activity will protect and expand lymphocyte numbers, which in turn will mediate potent anti-viral effects.

In view of the foregoing, the invention provides novel TPA compositions and methods that mediate clinical anti-viral benefits in subjects at risk for, or presenting, with SARS-COV-2 and other viruses. The subject compositions and methods will substantially reduce overall viral numbers, and lessen attendant adverse effects in treated subjects, by reducing viral load/titer in an upper or lower respiratory tract; reducing viral load/titer in non-respiratory tissues and organs (e.g., in subjects presenting with CSS or PIMS affecting multiple organs); reducing or preventing viral attachment and entry into cells; impairing viral replication in infected cells, and/or 5) inhibiting or blocking viral shedding from the respiratory tract.

### Anti-ARDS Compositions and Methods

In other aspects of the invention, mammalian subjects are administered an anti-ARDS effective amount of a TPA compound to elicit an anti-ARDS response in a subject presenting with an acute respiratory distress syndrome (ARDS), including subjects presenting with severe acute respiratory syndrome (SARS) mediated by a human SARS (hSARS) coronavirus. In exemplary embodiments, subjects amenable to TPA anti-ARDS treatment will present with a positive SARS-COV-2 nasopharyngeal or other test result indicative of active SARS-COV-2 infection, and treatment will begin within the first 1-8 days following initial COVID-19 disease symptoms (upper respiratory tract symptoms such as cough, sneezing, sore or itchy throat, and/or fever), and before severe lower respiratory COVID-19 disease develops. The subject compositions and methods are effective to reduce or eliminate one or more indicia of ARDS in treated versus control subjects, selected from: 1) dyspnea (shortness of breath); 2) hypoxemia; 3) Elevated level(s) of one or more pro-inflammatory cytokine(s) or other inflammatory factor(s) in the lung parenchyma (Lung parenchyma is the portion of the lung involved in gas transfer—the alveoli, alveolar ducts and respiratory bronchioles tissue) or; 2) Increased infiltration and/or elevated level(s) of monocytes, macrophages and/or neutrophils in the lung parenchyma and/or pulmonary alveolar airspaces; 3) Disruption of pulmonary endothelial and/or epithelial barriers; 4) Pathogenic fibrosis and/or other histopathologic indicia of immunogenic or hyperinflammatory disease injury in the lungs; 5) Elevated indicia of oxidative stress in a pulmonary tissue or other tissue, organ or biological sample from the subject (wherein each indicator/value is measured and determined in treated subjects, in comparison to the same indicator/value measured and determined in similar, placebo-treated control subjects). In certain embodiments of the invention, TPA compounds and methods herein exert anti-viral and other preventive and therapeutic activities against COVID-19, ARDS and CSS through activation of nuclear factor kappa-light-chain-enhancer of activated B cells (NFkB). NFkB is a protein complex that regulates DNA transcription and is a critical regulator of immune and inflammatory functions. The NFkB pathway mediates important cellular processes such as stress response, inflammation, and adaptive and innate immunity. Aberrant transcriptional regulation by NFkB has been linked to cancer and autoimmune diseases. It is now clear that proper regulation of NFkB is essential to healthy, balanced immune and inflammatory function as a whole, including by determining balanced activation, cellular determination (timing and course of immune cellular differentiation, cell fate, cell programming, signal-response capacity, including receptor expression and function, cellular lifespan and apoptosis) of important immune and inflammatory effector cells.

Latent, inactive NFkB dimers are present in the cytosol of most cells at all times. This allows NFkB to be a primary transcription factor responsive to viral and other stimuli, and a key component in the inflammasome cascade (Oeckinghaus et al., 2009). Once activated, NFkB primes the NLRP3-inflammasome for activation by inducing IL-1 beta and NLRP3 expression. The signaling adaptor p62/SQSTM1, via NFkB activation, has been identified as a positive mediator of Ras-induced inflammation and tumorigenesis (Duran et al., 2008), making it an interesting target for cancer therapies (Zhang et al., 2016). Researchers are now finding that NFkB also guards against excessive inflammation via a p62/SQSTM1-mediated mitophagy process. Specifically, NFkB restricts its own inflammation-promoting activity in macrophages by promoting p62/SQSTM1-mediated removal of damaged mitochondria (Zhong et al., 2016). This likely serves as a negative feedback loop to control inflammation reactions and prevent tissue damage.

According to the teachings herein, TPA compounds function as activators of NFkB in a novel manner that maintains downstream anti-viral effects, while down-regulating hyper-inflammatory effects in COVID-19, ARDS AND CSS treatment subjects. While NFkB functions initially as an early primer of inflammation, timely intervention with TPA at a point preceding an irreversible stage of hyper-inflammation (i.e., prior to onset of CSS) “activates” NFkB to down-regulate hyper-inflammation in macrophages and neutrophils via a novel mitophagy pathway. NFkB operates in this capacity to feedback-limit its own pro-inflammatory activity in macrophages, by promoting p62/SQSTM1-mediated removal of damaged mitochondria (Zhong et al., 2016). Pilot studies here indicate that when TPA-mediated NFkB activation is applied at the proper stage in COVID-19 and ARDS (e.g., when macrophages and neutrophils are activated but not yet hyperactivated, and before COVID-19 and ARDS-related tissue damage mediated by macrophages and neutrophils occurs), this regulation will clinically reduce or block hyper-inflammation, including CSS, and attendant tissue damage.

Another important aspect of TPA efficacy in treating human SARS viral infection, ARDS, CSS, PIMS and other viral-induced hyper-inflammatory and pathogenic conditions (e.g., in COVID-19 subjects), relates to the effect of TPA on immune- and inflammatory-regulating kinase activation, signaling and downstream inflammatory and pathogenic mechanisms. ACE2 is downregulated by attachment of SARS-COV-2, which leads to NFkB and mitogen activated protein kinase (MAPK) signaling pathways becoming hyper-activated, resulting in pulmonary inflammation and injury. These and other interactions between virus and host correlate with aberrant regulation of PKC, another regulator of MAPK activation and the MAPK pro-inflammatory signal-cascade. In general terms, TPA is capable of interacting with and activating PKC, resulting in substrate phosphorylation to propagates signals involved in MAPK inflammatory cascades.

More complex effects of TPA on MAPK pathways involve TPA regulation of immune cell differentiation and apoptosis. Our studies show that the exemplary TPA compound 12-O-tetradecanoylphorbol-13-acetate not only activates, but stabilizes PKC, effecting a clinically beneficial reduction in MAPK hyper-stimulation (reduced MAPK activation and phosphorylation activity, attenuating propagation of downstream MAPK-mediated pro-inflammatory signaling).

In certain aspects of the invention, pro-apoptotic effects of TPA compounds mediate increased apoptosis in MAPK stimulated target cells (including macrophages and neutrophils), further limiting the MAPK-mediated hyper-inflammatory activity of these target cells.

In related embodiments, pro-apoptotic effects of TPA compounds mediate increased apoptosis in pathogenic inflammatory cells that are hyper-stimulated and re-programmed through viral dysregulation of transforming growth factor beta (TGF-β). TGF-β is a multifunctional cytokine belonging to the transforming growth factor superfamily, which includes three mammalian isoforms (TGF-β 1 to 3, HGNC symbols TGFB1, TGFB2, TGFB3) and many other signaling proteins. TGF-β proteins are produced by all white blood cell lineages. When viral infection is present, activated TGF-β forms serine/threonine kinase complexes with other immune/inflammatory co-factors, which complexes bind TGF-β receptors. TGF-β receptors are composed of both type 1 and type 2 receptor subunits. After receptor binding of TGF-β, the type 2 receptor kinase phosphorylates and activates the type 1 receptor kinase, activating an immune/inflammatory signal-cascade. This potentiates synthesis and/or activation of diverse target genes and gene products that function in differentiation, chemotaxis, proliferation, activation, programming and apoptosis of immune and inflammatory cells.

TGF-β is secreted by many cell types, including macrophages, in a latent form complexed with two other polypeptides (latent TGF-beta binding protein (LTBP) and latency-associated peptide (LAP)). Serum proteinases such as plasmin catalyze the release of active TGF-β from the complex. This occurs on the surface of macrophages, where latent TGF-β complex is bound to CD36 via its ligand, thrombospondin-1 (TSP-1). Inflammatory stimuli, such as viral infection, that activate macrophages enhance the release of active TGF-β by promoting activation of plasmin. Macrophages also endocytose IgG-bound latent TGF-β complexes that are secreted by plasma cells, then release active TGF-β extracellularly.

A primary function of TGF-β is therefore as a pro-inflammatory cytokine. However, TGF-β also plays distinct roles in immune and inflammatory cell determination and differentiation. Only a few TGF-β activating pathways are currently known, and broader mechanisms behind TGF-β activation remain poorly understood. Known activators of TGF-β include proteases, integrins, pH, and reactive oxygen species (ROS).

It is well known that perturbations causing dysregulation of TGF-β activation and signaling can mediate hyper-inflammation, autoimmune disorders, fibrosis and cancer. This is well substantiated by rampant hyper-inflammation in TGF-β null transgenic mice. As described above, COVID-19 disease and other SARS viral infections cause profound pulmonary inflammation, fibrosis, and diffuse alveolar damage (DAD) in severe cases. Macrophages and neutrophils infiltrate in high numbers into the lung parenchyma and alveolar spaces, correlated with ARDS in severe COVID-19 patients. This pathology often extends to include neutrophil infiltration into pulmonary capillaries, deposition of extensive neutrophil extracellular traps (NETs), linked to lung fibrosis, thromboses and vasculitis. Similar mechanisms of hyper-inflammation manifesting as CSS are apparent in PIMS and late stage COVID-19 where CSS hyper-inflammation and pathogenesis may lead to multiple organ involvement and failure. Studying the original SARS-COV coronavirus, Zhao et al. (2008) reported that the SARS-COV nucleocapsid (N) protein actually potentiates TGF-β to mediated hyper-inflammation by certain pathways, while disabling a critical pathway/activity of TGF-β involved in inducing apoptosis in mature immune/inflammatory cells. The SARS N protein specifically induces expression of plasminogen activator inhibitor-1, while attenuating Smad3/Smad4-mediated apoptosis of human peripheral lung epithelial (HPL) cells. The hyper-activating effects of SARS N protein on TGF-β inflammatory cascades is Smad3-specific. N protein associates with Smad3 and promotes Smad3-p300 complex formation while it interferes with the complex formation between Smad3 and Smad4 (Zhao et al., 2008). These findings implicate a surprising mechanism whereby SARS viral N proteins disrupt and commandeer one specific pathway of TGF-β activation and signaling to deleteriously block normal apoptosis and extend the lifespan of SARS-COV-infected, pathogenic inflammatory host cells (while in parallel activating other mechanisms of TGF-β-mediated hyper-inflammation).

An important object of the invention is to prevent and curtail pulmonary fibrosis and other injury in COVID-19 subjects, mediated by a massive influx of activated macrophages and neutrophils into the lung parenchyma and alveolar spaces in severe cases. TPA compositions and methods of the invention achieve this clinical objective in part by reversing SARS N protein blockade of TGF-β-mediated apoptosis. This viral “re-programming” mechanism is believed to account for the extreme elevation in numbers of activated macrophages and neutrophils in COVID-19 subjects with severe CSS and ARDS symptoms. Whereas these inflammatory/immune cells would ordinarily undergo apoptosis after performing their anti-viral functions (phagocytosis and degranulation), COVID-19 recruits these cells by extending their lifespan (through N-protein suppression of TGF-β-induced apoptosis), thereby mis-regulating continued hyper-inflammation and enhanced viral replication and spread. This recruitment and protection of pathogenic macrophages and neutrophils by SARS coronaviruses is clearly part of the virus' etiologic and evolutionary strategy. Whereas circulating lymphocytes (T, B and NK cells) evidently lack ACE-2 receptors, ACE2-expressing CD68+CD169+ macrophages have been detected in COVID-19 patients, particularly in the splenic marginal zone and in marginal sinuses of lymph nodes, and these macrophages contained SARS-COV-2 nucleoprotein antigen and showed upregulation of IL-6 Feng et al., 2020). This indicates that macrophages can be infected by COVID-19 in the manner of a “trojan horse”, conveying the virus to vulnerable tissues and organs and contributing fundamentally to hyper-inflammation and activation-induced lymphocytic cell death during SARS-COV-2 infection.

Whether or not neutrophils express ACE-2 receptors and can be directly infected by hSARS viruses is unclear, but the role of ACE-2 in SARS-COV-2 hyper-inflammatory activation and potentiation of neutrophils is clear. Studies by Li et al. (2020) show that ACE2 is not only a receptor that facilitates SARS-COV-2 cellular entry and replication, but is also involved in post-infection viral-mediated dysregulation of host immune responses, cytokine expression and inflammation. ACE-2 levels in infected cells and tissues are correlated with severity of ARDS induced by the SARS-COV-2, and directly associated with hyper-elevation of pro-inflammatory cytokines mediating CSS and ARDS. More particularly, Li and colleagues report that high expression of ACE2 is related to intensity of innate immune responses, adaptive immune responses, B cell regulation and cytokine secretion, as well as hyper-inflammatory responses induced by IL-1, IL-10, IL-6 and IL-8. In other words, immune system dysfunctions that mediate CSS are broadly linked to high expression of ACE2. Additionally, high expression of ACE2 correlates with increased expression of genes involved in viral replication, and alteration of the transcriptome of SARS-COV-2-infected epithelial cells, enhancing viral entry, replication and assembly. T cell activation and inflammatory responses mediated by T cells are also induced by SARS-COV-2 alteration of the transcriptome in infected cells. Increased levels of IL1β, IFN-γ, IP10, and MCP1 in patients infected SARS-COV-2 appear linked to the hyper-activation of T-helper-1 (Th1) cell responses. ACE2 also mediates activation of neutrophils, NK cells, Th17 cells, Th2 cells, Th1 cells, dendritic cells and TNFα secreting cells, leading to a severe hyper-inflammatory response. High ACE2 expression in pulmonary tissue specifically cytotoxic activation of macrophages, neutrophil inflammation and a Th2-dominated immune response (Li et al., 2020). Intriguingly, ACE2 expression varies in a time-dependent manner after SARS-COV infection.

As a consequences of SARS-COV-2 viral “recruitment and reprogramming” of host macrophages and neutrophils, hijacked and “immortalized” inflammatory effector cells proceed to congest and impair the host's circulation, and degrade and congest interstitial and alveolar compartments (including through pulmonary fibrosis, NET deposition, epithelial and endothelial barrier destruction, vasculitis, and thromboses), fundamentally limiting the ability for the host to mount a successful immune response (e.g., by obstructing immune effector cells and secreted antibodies from reaching viral nursery sites in the lungs and other organs). The culmination of impacts from these dysregulated and hijacked processes is ARDS/SARS, attended by extreme pulmonary inflammatory pathogenesis, blockade of circulation and gas exchange, hypoxemia and eventual lung/heart failure. These are signature impacts of a novel (e.g., zoonotic), generalist virus, not the fine-tuned, attenuated impacts of a specialized virus long-coevolved with its host. By employing the TPA compositions and methods of the invention, SARS-COV-2 infective and pathogenic mechanisms are effectively blocked or substantially reduced. In exemplary embodiments, anti-inflammatory methods employing TPA compounds reduce SARS-COV-2 induced hyper-inflammation, CSS, ARDS, PIMS and related tissue and organ injuries that attend these pathogenic conditions. In certain embodiments, TPA compounds of the invention prevent or reduce SARS-COV-2 activation and lifespan extension (N protein disruption of normal apoptosis) in host macrophages and neutrophils, thereby limiting the pathogenic effects of CSS, ARDS, DAD, PIMS, and ESHS, and consequently limiting viral replication, viral spread within the host, viral shedding and viral transmission. Without wishing to be bound by theory, these anti-inflammatory and “pro-apoptotic” effects of TPA compounds and methods of the invention may be directly or indirectly linked to PKC, MAPK, NFkB and/or TGF-β-targets, mechanisms and pathways, or wholly independent therefrom.

According to the teachings herein, patients treated with anti-ARDS compositions and methods of the invention will show at least a 20% reduction, often a 25-50% reduction, in many cases a 75-95% or greater reduction, up to 100% elimination of one or more indices, conditions or symptoms correlated with ARDS severity, for example selected from dyspnea, elevated level(s) of one or more pro-inflammatory cytokine(s) in the lung, elevated level(s) of monocytes, macrophages and/or neutrophils in the lung parenchyma and/or pulmonary alveolar airspaces, disruption of pulmonary endothelial and/or epithelial barriers, elevated indicia of oxidative stress in the lung tissue (e.g., elevated levels of reactive oxygen species (ROS) in the lung, and/or one or more pathogenic symptom(s) of lung injury (e.g., hyper-inflammation, fibrosis, diffuse alveolar damage (DAD), macrophage and/or neutrophil infiltration into the lung parenchyma, macrophage and/or neutrophil infiltration into pulmonary capillaries, deposition of extensive neutrophil extracellular traps (NETs) in the lung interstitium, pulmonary and/or coronary vessel thromboses and vasculitis, among other pathologic indicia associated with ARDS injury). Efficacy of the claimed compositions and methods is determined for each of the foregoing indicators of ARDS by measuring and determining a clinical diagnostic value for the subject indicator in treated subjects, in comparison to the same indicator/value measured and determined in similar, placebo-treated control subjects.

The anti-ARDS compositions methods and compositions described herein are effective to treat or prevent one or more ARDS symptoms in a range of treated ARDS subjects, including ARDS caused by a viral pathogen (e.g., SARS), bacterial pathogen, caustic agent, pulmonary injury or trauma, burns and other causes.

### Anti-CSS Compositions and Methods

In further exemplary embodiments, mammalian subjects are administered an anti-CSS effective amount of a TPA compound to elicit an anti-CSS response in subjects infected with a SARS virus, presenting with COVID-19 disease, or otherwise exhibiting symptoms of cytokine storm syndrome (CSS). The rationale, strategy and mechanisms of activity for using TPA compounds to treat and prevent CSS in COVID-19 disease and other hyper-inflammatory conditions, generally follows the foregoing description for anti-ARDS compositions and methods. According to these aspects of the invention, patients treated with anti-CSS compositions and methods of the invention will show at least a 20% reduction, often a 25-50% reduction, in many cases a 75-95% or greater reduction, up to 100% elimination of one or more indices, conditions or symptoms correlated with CSS incidence and/or severity, for example: hyper-elevated pro-inflammatory cytokine activation, expression and/or levels in CSS-affected cells or tissues; increased infiltration and/or elevated numbers of macrophages and/or neutrophils in the lung parenchyma, pulmonary alveolar airspaces, or another CSS-affected tissue or organ; lymphocytopenia (numerical crashing of lymphocytes (T, B and NK cells)); elevated oxidative stress markers; inflammatory injury to endothelial and/or epithelial barriers in the lungs or other CSS-affected tissue or organ; pathogenic fibrosis and other pathologic inflammatory injury to lungs or other CSS-affected tissue or organ/organ; inflammatory injury, loss or atrophy of lymph nodes; inflammatory injury or atrophy of the spleen; sepsis; Toxic Shock Syndrome (TSS); and/or oxidative stress symptoms (wherein each indicator/value is measured and determined in treated subjects, in comparison to the same indicator/value measured and determined in similar, placebo-treated control subjects).

In more detailed aspects, the anti-CSS methods and compositions of the invention are surprisingly effective to reduce or eliminate a major causal factor in CSS, namely the dysregulation and hyper-elevation of pro-inflammatory cytokines beyond levels normally associated with beneficial inflammatory responses. According to the teachings herein, patients treated with anti-CSS compositions and methods of the invention will show at least a 20% reduction, often a 25-50% reduction, in many cases a 75-95% or greater reduction, up to 100% elimination of a hyper-elevated level of one or more pro-inflammatory cytokine(s) associated with CSS.

In exemplary embodiments, TPA-treated subjects will exhibit substantially reduced levels of a key pro-inflammatory cytokine, IL-6, associated with COVID-19-induced CSS. In illustrative working examples, levels of IL-6 in TPA-treated subjects presenting at outset of treatment with COVID-19 disease coupled with pro-inflammatory cytokine elevation and other CSS symptoms, as described herein, will show a therapeutic downregulation of IL-6 corresponding to at least a 20% reduction, 25-50% reduction, up to a 75-95% reduction in IL-6 levels in a target tissue or sample (e.g., a circulating blood sample, or lung tissue from biopsy or autopsy) compared to placebo treated control subjects presenting with comparable cytokine levels and CSS symptoms. For demonstration of prophylactic efficacy, test and control groups of patients will be selected for comparable CSS risk factors (e.g., all having early diagnosed COVID-19 disease), and after treatment is completed fewer patients in the TPA treatment group will develop CSS symptoms, including elevated IL-6 levels, and those who do will have less severe symptoms, including substantially lower IL-6 levels, compared to incidence and severity of CSS and hyper-stimulated levels of IL-6 in CSS positive, placebo-treated control subjects.

Comparable efficacy for preventing and reducing hyper-elevated pro-inflammatory cytokine levels associated with CSS will be achieved using TPA therapy to mediate reduced expression or levels in a target plasma, cell or tissue of a wide range of pro-inflammatory cytokine targets in addition to IL-6, including but not limited to: (IL)-1B; IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF). Each of these pro-inflammatory cytokine targets for TPA-mediated attenuation is reported by Huang et al. (2020) to be abnormally elevated in patients with serious COVID-19 disease. In severe ICU patients, Huang and coworkers report that IL-2, IL-7, IL-10, G-CSF, IP10, MCP1, MIP1A, TNFα were higher than in the non-ICU patients (Huang et al., 2020; Conti et al., 2020). Within exemplary embodiments of the invention, TPA therapy is targeted to reduce expression, activation, half-life and/or levels of one or more CSS-related pro-inflammatory cytokine(s) selected from: IL-10 (inhibits inflammatory cytokine production by monocytes/macrophages and neutrophils, and inhibits TH1-type lymphocyte responses); IL-11 (inhibits proinflammatory cytokines response by monocyte/macrophages, additionally promotes Th2 lymphocyte response); IL-13 (attenuates monocyte/macrophage function), IL-37 and other pro-inflammatory cytokines and chemokines implicated in CSS.

Without wishing to be bound by theory, TPA compositions and methods of the invention potently attenuate hyper-activation and hyper-expression of pro-inflammatory cytokines attending CSS indirectly, at the cellular level (e.g., through reduction of activation-induced lymphocytopenia, resulting in attenuation of normal T, B and NK cellular functions), and via signal-response pathways described above involving PKC, MAPK, NFkB, TGF-β and other targets, mechanisms and pathways associated with normal immune and inflammatory regulation. In other exemplary embodiments, TPA-treated subjects will exhibit substantially elevated levels of anti-inflammatory cytokines, which novel effect in turn mediates further reduction of CSS symptoms and pathology, including in COVID-19 disease subjects. In illustrative working examples, levels of anti-inflammatory cytokines in TPA-treated subjects presenting at outset of treatment with COVID-19 disease and associated pro-inflammatory cytokine elevation and other CSS symptoms as described herein, will show a therapeutic upregulation of one or more anti-inflammatory cytokine(s), corresponding to a 20% increase, 25-50% increase, up to a 75-95% increase in the cytokine(s) level(s) in a target tissue or sample (e.g., a circulating blood sample, or lung tissue from biopsy or autopsy) compared to placebo treated control subjects presenting with comparable pro-inflammatory cytokine levels and CSS symptoms. For demonstration of prophylactic efficacy, test and control groups of patients will be selected for comparable CSS risk factors (e.g., all having early diagnosed COVID-19 disease), where after fewer patients in the TPA treatment group will develop CSS symptoms, including elevated pro-inflammatory cytokine levels, and those who do will have less severe symptoms, correlated with substantially elevated anti-inflammatory cytokine levels, and substantially reduced pro-inflammatory cytokine levels, compared to incidence and severity of CSS and anti- and pro-inflammatory cytokine profile values in placebo-treated control subjects.

Comparable efficacy for upregulating anti-inflammatory cytokines, to prevent and reduce CSS symptoms, including hyper-elevated pro-inflammatory cytokine levels, will be achieved using TPA therapy for a wide range of anti-inflammatory cytokine targets, including but not limited to: IL-1RA (an interleukin secreted by pro-immune cells and epithelial cells, which inhibits pro-inflammatory effects of IL1β and modulates a variety of interleukin 1 related immune and inflammatory responses); soluble TNF receptors (sTNFRs), including sTNFR1 (circulating counterpart of membrane bound TNFR1 (mTNFR1), which binds to TNF trimers in the circulation, preventing membrane-bound TNF receptor-TNF ligand interactions), and sTNFR2 (counterpart of mTNFR2, which binds to TNF trimers in the circulation, preventing membrane-bound TNF receptor-TNF ligand interactions); Soluble IL-1 receptor type 2 (sIL-1RII) (which binds to circulating IL-1 ligands in the plasma, preventing IL-1β from binding to the IL-1 receptor type 1); membrane-bound IL-1 receptor type 2 (mIL-1RII) (which functions as a “decoy” receptor lacking intracellular signaling function but competing with type 1 IL-1R for IL-1 ligand binding at the cell membrane); IL-10 (inhibits Th1 cytokines, including IL-2 and IFN-g, deactivates monocyte/macrophage proinflammatory cytokine synthesis, and down-regulates or inhibits monocyte/macrophage-derived TNF-a, IL-1, IL-6, IL-8, IL-12, granulocyte colony-stimulating factor, MIP-1a, MIP-2a, IL-18BP); IL-11 (attenuates IL-1 and TNF synthesis in macrophages by up-regulating inhibitory NF-kB (inhibitory NF-kB), which blocks nuclear translocation of NF-kB and thus impairs transcriptional activation of proinflammatory cytokines, inhibit synthesis of IFN-g and IL-2 by CD41 T cells, functions as a Th2-type cytokine, inhibits cytokine expression by Th1 lymphocytes); IL-13 (down-regulates production of TNF, IL-1, IL-8, and MIP-1a by monocyte/macrophage cells); and TGF-β (has both pro- and anti-inflammatory effects, serves as biological switch to antagonize, potentiate or modify actions of other cytokines and growth factors, is capable of converting an active site of inflammation into one dominated by resolution and repair, may function as immune-enhancer locally, and immune-suppressor in systemic circulation, suppresses proliferation and differentiation of T cells and B cells; downregulates IL-2, IFN-g, and TNF; serves as monocyte/macrophage deactivator in a manner similar to IL-10, induces apoptosis in mature immune/inflammatory cells).

In related embodiments of the invention directed to treatment and prevention of CSS, TPA compositions and methods described herein effectively block or reduce infiltration and numerical increase macrophages and/or neutrophils in the lung parenchyma, pulmonary alveolar airspaces, or another CSS-affected tissue or organ. These effects of TPA compounds can involve a variety of mechanisms, including blocking or reducing of pro-inflammatory cytokine and/or chemokine signaling of macrophages and/or neutrophils (to block or reduce their activation, migration/infiltration, blocking or reducing macrophage and/or neutrophil pro-inflammatory cytokine expression, cytotoxicity, destruction of epithelial and endothelial cells and structural components, blocking or reducing NET deposition by activated neutrophils, and blocking or reducing pro-thrombogenic activities and related pathogenic effects of macrophages and/or neutrophils. Additionally, the TPA compositions and methods of the invention mediate pro-apoptotic effects on macrophages and neutrophils, positively regulating their normal inflammatory activity and timely apoptosis (and in the case of COVID-19 disease, subverting SARS-COV-2 recruitment and re-programming of apoptosis in these cells, mediated by SARS N protein effects on TGF-β that pathogenically impairs normal apoptosis to extend the lifespan and hyper-inflammatory effects of these cells).

In additional aspects of the invention relating to CSS treatment and prevention, the TPA compounds and methods of the invention are also clinically effective to treat or prevent lymphocytopenia. Lymphocytopenia is a profound reduction in numbers of lymphocytes (T, B and NK cells) in the blood, spleen and/or lymph nodes observed in severe CSS cases (i.e., cases that progress to tissue/organ pathogenesis, injury and dysfunction) mediated at least in part by activation-induced lymphocytic cell death.

In other embodiments the TPA compositions and methods of the invention are effective to treat or prevent oxidative stress associated with CSS, for example as determined by reduction in oxidative stress markers (e.g., elevated/ROS levels) in a pulmonary tissue or other CSS-affected tissue or organ.

Other treatment targets of the invention associated with CSS include prevention or reduction of inflammatory injury to endothelial and/or epithelial barriers in the lungs or other CSS-affected tissue or organ. Diagnostic indicia of efficacy in these working examples include known histological examination and histochemical assays evaluating biopsy and/or autopsy results between treated and control groups of CSS subjects.

Related TPA treatment methods and compositions of the invention are directed toward prevention and treatment of pathogenic fibrosis and/or other histopathologic indicia of immunogenic or hyperinflammatory disease injury in the lungs or other CSS-affected tissue or organ/organ, disappearance and/or atrophy of lymph nodes, and inflammation and/or atrophy of the spleen—all of which are assessed for efficacy employing conventional pathologic exam, histology and histochemical methods.

Yet additional embodiments of the invention are directed to CSS prevention and treatment involving reduction or blocking of sepsis, toxic Shock Syndrome (TSS), and/or oxidative stress symptoms associated with CSS. Here too, conventional assay methods are widely known in the art for measuring diagnostic parameters for assessment of treatment/prophylactic efficacy. symptoms.

### Anti-Inflammatory and Immune-Regulatory Compositions and Methods

Additional embodiments of the invention TPA compositions and methods that prevent or treat a hyper-immune or hyper-inflammatory condition, including but not limited to a Pediatric Inflammatory Multisystem Syndrome (PIMS) associated with COVID-19 disease, Kawasaki disease, Extrapulmonary Systemic Hyperinflammation Syndrome (ESHS), or a vascular congestive or thrombotic condition caused by hyperinflammation. The vascular congestive or thrombotic condition may include, but is not limited to, Disseminated Intravascular Coagulation (DIC), thrombosis, stroke, thrombocytopenia, and/or gangrene. When these compositions and methods are administered to human subjects at elevated risk or presenting with a hyperimmune or hyperinflammatory condition or disorder, clinical efficacy will be demonstrated by a substantial reduction (i.e., at least a 20% reduction, a 25-50% reduction, a 75-95% reduction, up to 100% prevention/elimination) of one or more targeted hyperimmune or hyperinflammatory condition or symptom selected from: 1) Elevated levels of one or more pro-inflammatory cytokine(s) or other inflammatory factor(s) in an affected tissue, organ or plasma of the subject; 2) Increased infiltration and/or elevated levels of monocytes/macrophages and/or neutrophils in an affected tissue, organ or compartment (e.g., alveolar, renal or vascular lumen) of the subject; 3) Disruption of endothelial and/or epithelial barriers of an affected tissue or organ of the subject; 4) Pathogenic fibrosis and/or other histopathologic indicia of immunogenic or hyperinflammatory disease injury in an affected tissue or organ of the subject; 5) Toxic Shock Syndrome (TSS) symptoms; 6) Elevated indicia of oxidative stress in an affected tissue, organ or biological sample from the subject; and/or 7) one or more Pediatric Inflammatory Multisystem Syndrome (PIMS)-associated symptoms selected from, sudden onset fever, rash, red eyes, dry or cracked mouth, redness in the palms of hands and/or soles of feet, swollen glands, swollen blood vessels, and/or coronary artery aneurysm (wherein each indicator/value is measured and determined in treated subjects, in comparison to the same indicator/value measured and determined in similar, placebo-treated control subjects).

Within related aspects of the invention, TPA is administered to a human subject to prevent or treat a hyper-immune or hyper-inflammatory condition, including but not limited to a Pediatric Inflammatory Multisystem Syndrome (PIMS) associated with COVID-19 disease, Kawasaki disease, Extrapulmonary Systemic Hyperinflammation Syndrome (ESHS), or a vascular congestive or thrombotic condition caused by hyperinflammation. The vascular congestive or thrombotic condition may include, but is not limited to, Disseminated Intravascular Coagulation (DIC), thrombosis, stroke, thrombocytopenia, and/or gangrene. According to these aspects, human subjects at elevated risk or presenting with a hyperimmune or hyperinflammatory condition or disorder are administered an immune modulatory or anti-inflammatory effective amount of a TPA compound. sufficient to reduce or prevent a targeted hyperimmune or hyperinflammatory condition or symptom selected from: 1) Elevated levels of one or more pro-inflammatory cytokine(s) or other inflammatory factor(s) in an affected tissue, organ or plasma of the subject; 2) Increased infiltration and/or elevated levels of monocytes/macrophages and/or neutrophils in an affected tissue, organ or compartment (e.g., alveolar, renal or vascular lumen) of the subject; 3) Disruption of endothelial and/or epithelial barriers of an affected tissue or organ of the subject; 4) Pathogenic fibrosis and/or other histopathologic indicia of immunogenic or hyperinflammatory disease injury in an affected tissue or organ of the subject; 5) Toxic Shock Syndrome (TSS) symptoms; 6) Elevated indicia of oxidative stress in an affected tissue, organ or biological sample from the subject; and/or 7) one or more Pediatric Inflammatory Multisystem Syndrome (PIMS)-associated symptoms selected from, rash, red eyes, dry or cracked mouth, redness in the palms of hands and/or soles of feet, swollen glands, swollen blood vessels, and/or coronary artery aneurysm (wherein each indicator/value is measured and determined in treated subjects, in comparison to the same indicator/value measured and determined in similar, placebo-treated control subjects).

### Combination Drug Therapy and Coordinate Treatment Methods

Within additional aspects of the invention, combinatorial formulations and coordinate treatment methods are provided that employ an effective amount of a TPA compound and one or more “secondary agent(s)”. The secondary agent may be a “secondary therapeutic agent”, or “secondary prophylactic agent”, and can be co-formulated or coordinately administered with the TPA compound, to yield a combined formulation, combination drug therapy or coordinate treatment or prophylactic method that is effective to treat or prevent a targeted COVID-19 infection or disease, ARDS generally, SARS, CSS, PIMS or other targeted infection, disease, condition and/or symptom described herein. For example, exemplary combinatorial formulations and coordinate treatment/prevention methods for ARDS comprise an anti-ARDS effective TPA compound combined with one or more secondary or adjunctive treatment or prophylaxis agents effective for treating and/or preventing ARDS, or a targeted, comorbid disease, condition or symptom. In alternative aspects, the secondary agent can possess the same, similar, or distinct pharmacological activity as the TPA compound for treating and/or preventing a targeted or co-morbid condition.

In related embodiments any TPA compound disclosed herein can be employed in drug combinations or combination therapy with one or more secondary therapeutic or prophylactic agents to treat and/or prevent any infection, disease, condition and/or symptom described herein. In illustrative embodiments, an anti-viral effective TPA compound is combined with, or coordinately administered with, a conventional anti-viral drug. In other exemplary embodiments, an anti-ARDS, anti-CSS, anti-PIMS, anti-ESHS, anti-DAD, anti-inflammatory, pro-immune, anti-cytopathic and/or pro-apoptotic TPA compound is combined with, or coordinately administered with, a secondary agent that mediates significant clinical benefit toward preventing or treating any disease, condition, symptom associated with, or comorbid in a selected patient with, viral infection, hyper-inflammation, ARDs, CSS, PIMS, ESHS, DAD, elevated cytopathic activity, immunosuppression, dysfunction of normal apoptotic activity of immune and/or inflammatory cells, and/or any other target symptom(s), condition(s) or index (ices) described herein, in treated subjects.

TPA compounds of the invention can be administered concurrently or sequentially with the secondary agent, and the secondary agent will act additively, synergistically or distinctly to treat and/or prevent the same, or different, disease, condition(s) or symptom(s) for which the TPA compound is administered. The TPA compound and the secondary agent may be combined in a single formulation, or separately administered at the same or different time. Thus, administration of the TPA compound and secondary agent may be done simultaneously or sequentially in either order, and a therapeutic interval may include time(s) when only one or both (or all) of the TPA compound and secondary therapeutic agent individually and/or collectively exert their therapeutic effect. A distinguishing aspect of all such coordinate treatment methods is that the selected TPA compound exerts at least some detectable therapeutic activity toward alleviating or preventing the targeted disease, condition(s) or symptom(s), as described herein, and elicits a favorable clinical response, which may or may not be in conjunction with a separate or enhanced clinical response mediated by the secondary therapeutic agent. In other illustrative embodiments, coordinate administration of the TPA compound with a secondary therapeutic agent will typically yield a greater therapeutic response compared to clinical responses observed following administration of either the TPA compound or the secondary agent alone at the same dosage, with reduced side effects. For example, coordinate treatment using an anti-viral effective TPA compound in combination with a conventional anti-viral drug may yield substantial therapeutic effects even when individually subtherapeutic doses of both the TPA compound and conventional anti-viral drug are administered, avoiding or lessening associated side effects (i.e., compared to the side effects observed using an equally therapeutic dose of the TPA compound or conventional anti-viral drug alone). In this manner, the TPA compound and conventional anti-viral drug are “potentiating” toward one another, eliciting combinatorial therapeutic efficacy at dosages that neither drug alone yields detectable therapeutic benefits. The surprising combinatorial efficacy of TPA with normally (i.e., when administered solo) sub therapeutic doses of complementary or potentiating secondary therapeutic drugs provides important advantages in terms of eliminating or substantially reducing the adverse side effects that may attend individual administration of full therapeutic doses of the TPA or secondary therapeutic drug. These coordinate dosage regimes employed here will vary, for example according to well-known clinical and patient-specific parameters, while the terms “therapeutic dose” and “sub therapeutic dose” have ordinary and clear meaning to persons skilled in the art (and are here applicable to any one or combination of symptoms associated with the targeted diseases, conditions and symptoms.

Anti-Viral Drug Combinations with TPA

In coordinate therapies of the invention where the secondary agent is an anti-viral drug, the secondary agent may be a conventional anti-viral. For example, the anti-viral drug may be selected from any one or combination of: Abacavir, Acyclovir, Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and/or Zidovudine, among many other safe and effective anti-virals that are known and readily available for clinical use. Anti-viral agents may also be selected from monoclonal antibodies and other biologics that immunospecifically bind to or otherwise disable a target virus, and steroids and corticosteroids, such as prednisone, cortisone, fluticasone and glucocorticoid.

In exemplary combinatorial compositions and methods of the invention, an anti-SARS-COV-2 effective TPA compound is co-formulated and delivered with (e.g., in a multi-drug iv infusion), or simultaneously or sequentially co-administered with, remdesivir. Remdesivir (GS-5734) is currently the promising anti-COVID-19 drug, that exhibits broad-spectrum antiviral activities against RNA viruses. It is a prodrug whose structure resembles adenosine. Remdesivir incorporates into nascent viral RNA and also inhibit the RNA-dependent RNA polymerase. This results in premature termination of the viral RNA chain and consequently halts replication of the viral genome. Remdesivir was originally developed by Gilead Sciences (USA) against the Ebola virus, and underwent clinical trials during the recent Ebola outbreak in the Democratic Republic of Congo. Although it was not proven effective against Ebola in this trial, its safety for humans was established, permitting its entry for ongoing clinical trials to determine remdesivir efficacy against COVID-19 disease. Importantly, remdesivir was previously shown to exhibit antiviral activities against different coronaviruses, including SARS-COV and MERS-COV, in vitro and in vivo. In a recent in vitro study, remdesivir was also reported to inhibit SARS-COV-2 (Wang et al., 2020). Remdesivir is now being tested in multiple trials in different countries, including two randomized phase III trials in China (NCT04252664 and NCT04257656).

In other exemplary combinatorial compositions and methods of the invention, an anti-SARS-CoV-2 effective TPA compound is co-formulated and delivered with, or simultaneously or sequentially co-administered with, favipiravir. Like remdesivir, favipiravir inhibits viral RNA-dependent RNA polymerase by structurally mimicking the endogenous guanine. Through competitive inhibition, efficacy of viral replication can be substantially impaired. Favipiravir has been approved for treatment for influenza, though less preclinical support has been established for favipiravir to treat SARS-COV-2 than for remdesivir. A clinical study in China evaluated efficacy of favipiravir plus interferon-α to treat SARS-COV-2 (ChiCTR2000029600), and in March 2020, favipiravir was approved as the first safe and effective anti-COVID-19 drug by the National Medical Products Administration of China.

In other exemplary combinatorial compositions and methods of the invention, an anti-SARS-CoV-2 effective TPA compound is co-formulated and delivered with, or simultaneously or sequentially co-administered with, Ivermectin. Ivermectin is an FDA-approved anti-parasitic drug, proven to exert antiviral activities toward both human immunodeficiency virus (HIV) and dengue virus. Ivermectin can dissociate preformed IMPα/β1 heterodimer, which is responsible for nuclear transport of viral protein cargos. As nuclear transport of viral proteins is essential for the replication cycle and inhibition of the host's antiviral response, targeting the nuclear transport process may be a viable therapeutic approach toward RNA viruses. Recently, an in vivo study has proven Ivermectin's capability to reduce viral RNA up to 5,000-fold after 48 h of infection with SARS-COV-2 (Caly et al., 2020). With an established safety profile for anti-parasitic use, the next step to prove Ivermectin's efficacy for treating COVID-19 will involve trials to determine optimal dosing.

In other exemplary combinatorial compositions and methods of the invention, an anti-SARS-CoV-2 effective TPA compound is co-formulated and delivered with, or simultaneously or sequentially co-administered with, lopinavir and ritonavir. The human immunodeficiency virus (HIV) protease inhibitors lopinavir and ritonavir are likely candidates within the TPA methods and compositions of the invention for anti-viral treatment of COVID-19 subjects. Aspartyl protease is an enzyme encoded by the pol gene of HIV that cleaves precursor polypeptides in HIV, thus playing an essential role in its replication cycle. Although coronaviruses encode a different enzymatic class of protease (a cysteine protease), evidence suggests that lopinavir and ritonavir will also inhibit the coronaviral 3CL1pro protease. A number of clinical, animal, and in vitro model studies performed on SARS and MERS established that the lopinavir/ritonavir combination of protease inhibitors was against these respective viruses. The lopinavir/ritonavir combination is presently in clinical trials for use in COVID-19 patients (NCT04252885; ChiCTR2000029308), though no clear benefits beyond Clay standard care have yet been reported.

Anti-ACE2 Drug Combinations with TPA

In other exemplary combinatorial compositions and methods of the invention, an anti-SARS-CoV-2 effective TPA compound is co-formulated and delivered with, or simultaneously or sequentially co-administered with, recombinant Human Angiotensin-converting Enzyme 2 (APN01). The soluble recombinant human Angiotensin-converting Enzyme 2 (rhACE2) is expected to block entry of SARS-COV-2 by blocking the S protein from interacting with the cellular ACE2. Indeed, in a recent study, it was reported that rhACE2 could inhibit SARS-COV-2 replication in cellular and embryonic stem cell-derived organoids by a factor of 1,000-5,000 times (Monteil et al., 2020). rhACE2 likely decreases serum angiotensin II by directing the substrate away from the related enzyme, ACE. This may prevent further activation of ACE2 receptor and thereby preserve pulmonary vascular integrity and prevent ARDS. APN01, originally developed by Apeiron Biologics, has already undergone phase II trial for ARDS. A small pilot study in China (NCT04287686) is now evaluating the biological and physiological role of rhACE2 in COVID-19 pneumonia, particularly as a treatment of ARDS. Apeiron Biologics has also initiated a placebo controlled, double blinded, dose-escalation study to access the safety and tolerability of intravenous APN01. By measuring plasma levels of angiotensin II and angiotensin 1-7, the bioproducts interfered with by the drug, and the biological and physiological roles of rhACE2 in COVID-19 pneumonia, will be further elucidated.

### Viral Entry Inhibitors

In other exemplary combinatorial compositions and methods of the invention, an anti-SARS-CoV-2 effective TPA compound is co-formulated and delivered with, or simultaneously or sequentially co-administered with, arbidol. Arbidol is a viral entry inhibitor developed against influenza and arboviruses. This drug targets hemagglutinin (HA), the major glycoprotein on the surface of influenza virus. Arbidol prevents fusion of the viral membrane with the endosome after endocytosis. Currently, it is undergoing trials as a single agent against SARS-COV-2 (NCT04260594, NCT04255017). Another clinical trial is directed toward comparing arbidol with favipiravir against SARS-COV-2 (ChiCTR2000030254).

In other exemplary combinatorial compositions and methods of the invention, an anti-SARS-CoV-2 effective TPA compound is co-formulated and delivered with, or simultaneously or sequentially co-administered with, an interferon. Clinical trials have recently been registered to evaluate a combination of lopinavir/ritonavir and IFNα2b (ChiCTR2000029387) or a combination of lopinavir/ritonavir with ribavirin and IFNβ1b administered subcutaneously (NCT04276688) for the treatment of COVID-19. IFN administration by vapor inhalation is currently part of standard of care COVID-19 treatment in China, offering the advantage of specifically targeting the respiratory tract. Intravenous and subcutaneous modes of IFN administration are well-described and have proven safe in several clinical trials. The combination of IFN-I with lopinavir/ritonavir, ribavirin or remdesivir could improve its efficacy, based on the enhanced efficacy of these combinations reported for other coronaviruses (Sheahan et al., 2020). Type III IFN is also a candidate for treatment of COVID-19, by virtue of its known protective effects in the respiratory tract (Lokugamage et al., 2020). Subcutaneous IFNβ1a in combination with lopinavir/ritonavir is being compared to lopinavir/ritonavir alone, hydroxychloroquine, and remdesivir in the DisCoVeRy trial (NCT04315948), which is the first clinical trial of the WHO Solidarity consortium of clinical trials.

Anti-Inflammatory Drug Combinations with TPA

In addition to anti-viral drug combinations and coordinate treatment methods, TPA compounds will be beneficially combined with secondary therapeutic and/or prophylactic agents to treat and/or prevent all other diseases, conditions and symptoms described herein as targets for therapeutic intervention. For example, “anti-inflammatory” drugs and biologics will be beneficially combined with TPA compounds in anti-ARDS, anti-CSS, anti-PIMS, anti-ESHS, anti-DAD, anti-inflammatory, pro-immune, anti-cytopathic and/or pro-apoptotic drug mixtures, coordinate administration protocols, and complementary multi-drug treatment methods, as combinatorially effective (e.g., complementary, additive, synergistic, potentiating) to coordinately treat one or more disease(s), condition(s) or symptom(s) associated with, or comorbid in a treated patient with, viral infection, hyper-inflammation, ARDs, CSS, PIMS, ESHS, DAD, elevated cytopathic activity, immunosuppression, dysfunction of normal apoptotic activity of immune and/or inflammatory cells, and/or any other target symptom(s), condition(s) or index (ices) described herein.

Within various embodiments of the invention, a wide variety of anti-inflammatory agents will be useful, including but not limited to: anti-inflammatory cytokines, steroids, corticosteroids, glucocorticoids, non-steroidal anti-inflammatory drugs (NSAIDs), antioxidants, prostaglandins, and antibiotics, among other anti-inflammatory agents.

In exemplary combinatorial compositions and methods of the invention, an anti-inflammatory effective TPA compound is co-formulated and delivered with, or simultaneously or sequentially co-administered with a non-steroidal anti-inflammatory drugs (NSAIDs). Illustrative NSAID candidates for use in these embodiments include, but are not limited to, aspirin, celecoxib (Celebrex), diclofenac (Cambia, Cataflam, Voltaren-XR, Zipsor, Zorvolex), diflunisal, etodolac, ibuprofen (Motrin, Advil), indomethacin (Indocin), celecoxib (Celebrex), piroxicam (Feldene), indomethacin (Indocin), meloxicam (Mobic Vivlodex), ketoprofen (Orudis, Ketoprofen ER, Oruvail, Actron), sulindac (Clinoril), diflunisal (Dolobid), nabumetone (Relafen), oxaprozin (Daypro), tolmetin (Tolmetin Sodium, Tolectin), salsalate (Disalcid), fenoprofen (Nalfon), flurbiprofen (Ansaid), ketorolac (Toradol), meclofenamate, mefenamic acid (Ponstel), among other safe and effective NSAID anti-inflammatory drugs widely known and available for clinical use.

Cytokine Inhibitor Drug Combinations with TPA

In other exemplary combinatorial compositions and methods of the invention, an anti-inflammatory effective TPA compound is co-formulated and delivered with, or simultaneously or sequentially co-administered with an anti-inflammatory drug or agent that directly or indirectly inhibits/lowers the induction, synthesis, activation and/or circulating level(s) of one or more pro-inflammatory cytokine targets that are hyper-elevated in association with SARS-COV-2 infection, COVID-19 disease, ARDS, SARS, CSS, PIMS, ESHS, DAD or any other hyper-inflammatory condition or symptom described herein. Within exemplary embodiments, an anti-inflammatory effective TPA compound will be co-formulated or coordinately administered with one or more secondary therapeutic agents that inhibit(s)/lower(s) the induction, synthesis, activation and/or circulating level(s) of one or more pro-inflammatory cytokine targets, selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF). Within these aspects of the invention, the TPA compound and secondary therapeutic agent are combinatorially effective to reduce expression, activation, half-life and/or levels of the one or more targeted pro-inflammatory cytokine(s), often with reduced side effects compared to administration of comparably therapeutic doses of the individual drugs.

Illustrative anti-inflammatory drug candidates for use as secondary therapeutic agents within these embodiments include above-described anti-inflammatory cytokines, as well as a variety of drugs and biological agents that specifically, directly or indirectly, target, bind, block, deactivate, and/or inhibit synthesis or activity of one or more pro-inflammatory cytokines.

Anti-Il-6 Biologics and Drug Combinations with TPA

Exemplifying these aspects of the invention, TPA compounds are combinatorially effective when used in combination with anti-IL-6 drugs or biologics, to substantially reduce the induction, synthesis, activation and/or circulating level(s) of IL-6. IL-6 has long been regarded as a keystone pro-inflammatory cytokine involved in pro-inflammatory cascades, along therapeutic with TNF-a and IL-1. In the case of IL-6, this cytokine is considered a marker for systemic activation of inflammatory effectors. Like many other cytokines, IL-6 has both pro-inflammatory and anti-inflammatory properties. Of particular importance, IL-6 is a potent inducer of acute-phase inflammatory responses, and elevated IL-6 levels are strongly correlated with poor prognosis in COVID-19 patients with ARDS (hyper-elevation of IL-6 is strongly associated with the need for mechanical ventilation). The classical pathway of IL-6 signaling occurs through IL-6 receptors, which are expressed on neutrophils, monocytes, macrophages, and other leukocyte populations. Besides binding to the membrane-bound IL-6 receptor (mIL-6R, CD126), IL-6 can also bind to the soluble form of IL-6 receptor created by proteolytic cleavage of mIL-6R or alternative splicing of mRNA. An elevated level of circulating IL-6 is associated with a faster decline of lung elasticity and more severe bronchoalveolar inflammation. Hence, specific blockade of IL-6-regulated signaling pathways represents a promising approach to attenuate acute inflammation associated with pulmonary damage in COVID-19 disease subjects.

In one exemplary embodiment targeting IL-6 with a multi-drug TPA strategy, a TPA compound is coordinately administered with an anti-IL inhibitor, binding agent or deactivating agent, such as a specific, anti-IL-6 monoclonal antibody or Fab fragment or a soluble IL-6 receptor or receptor analog, and related biologics comprising cognate anti-IL-6 binding or deactivating domains thereof.

In certain embodiments, an anti-IL-6 monoclonal antibody, siltuximab, made by EUSA Pharma, is employed in coordinate anti-inflammatory treatment methods with a TPA compound. Siltuximab is currently under clinical investigation for use in treating COVID-19 patients with ARDS.

In other embodiments, sarilumab (Kevzara), an IL-6 receptor antagonist, is employed with an anti-inflammatory TPA compound to disable or impair IL-6 activity and thereby mediate clinical benefits in subjects with COVID-19, ARDS, CSS and other hyper-inflammatory conditions. Kevzara has proven efficacy in treating arthritis, and Regeneron Pharmaceuticals and Sanofi are currently conducting phase II and a phase III trials on Kevzara in severe and critical COVID-19 patients (NCT04315298).

In other embodiments, tocilizumab (TCZ), a recombinant human anti-IL-6 monoclonal antibody is employed with an anti-inflammatory TPA compound to disable or impair IL-6 activity and effect clinical benefits in subjects with COVID-19, ARDS, CSS and other hyper-inflammatory conditions. TCZ specifically binds to soluble and membrane-bound IL-6 receptors (IL-6Rs), thus blocking IL-6 signaling and IL-6-mediated inflammatory responses. TCZ has been widely used in rheumatic diseases, such as rheumatoid arthritis. In 2017 TCZ was approved in the US for severe life-threatening CSS caused by chimeric antigen receptor T-cell (CART) immunotherapy. A current study is evaluating the effects of TCZ in treatment of severe and critical COVID-19 patients. Preliminary reports indicate that TCZ reduces fever and other ARDS symptoms, and 75.0% of treated subjects showed improved oxygenation. Opacity lung lesion on CT scans absorbed in 90.5% patients. In addition, peripheral lymphocyte levels returned to normal in 52.6% patients. Several current clinical trials are registered to evaluate safety and efficacy of tocilizumab in the treatment of severe COVID-19 pneumonia in adult inpatients, including a multicenter, randomized controlled trial for the efficacy and safety of tocilizumab in novel coronary pneumonia (NCP) patents (ChiCTR2000029765), a single arm open multicenter study on tocilizumab (ChiCTR2000030796), and a study on tocilizumab in combination with anti-viral drugs (ChiCTR2000030442 and ChiCTR2000030894).

In related embodiments, an anti-inflammatory effective TPA compound is co-formulated and delivered with, or simultaneously or sequentially co-administered with, an anti-IL-6 drug that blocks or inhibits IL-6 directly, or indirectly inhibits, lowers, or alters a pro-inflammatory activity of IL-6. One such drug is andrographolide, originally identified in the plant Andrographis paniculata. Andrographolide is a diterpenoid labdane compound that is the main bioactive component found in a traditional medicinal plant, Andrographis paniculata, that widely used in China and India for treating infection, inflammation, cold, fever, pneumonia and other conditions, and diarrhea in India and China. Andrographolide exhibits a wide spectrum of documented biological activities of therapeutic importance, including antibacterial, anti-inflammatory, antimalarial, and anticancer activities. Andrographolide exhibits strong anti-inflammatory activity, including via inhibition of NF-κB signaling. Andrographolide also suppresses inducible nitric oxide synthase and production of reactive oxygen species associated with hyper-inflammation. Andrographolide can additionally induce cell cycle arrest by increasing expression of p27 and decreasing expression of cyclin-dependent kinases, and trigger apoptosis via caspase-8-dependent pathways. In murine peritoneal macrophages, andrographolide inhibits the production of TNF-α and interleukin-12 via suppression of the ERK1/2 signaling.

Andrographolide Drug Combined with TPA

With respect to IL-6-mediated inflammation, andrographolide has also been shown to have potent anti-inflammatory activity affecting IL-6-mediated hyper-inflammatory mechanisms and pathways. Andrographolide inhibits IL-6 production and suppresses IL-6 hyper-inflammatory signaling in a dose-dependent manner, both in vitro and in vivo (including within Stat3, Akt, and ERK1/2 pathways) (Chun, 2010). According to exemplary embodiments of the invention, coordinate administration of an anti-inflammatory effective TPA compound with andrographolide is combinatorially effective to block or inhibit IL-6 activity, often by multiple pathways and/or mechanisms, directly, or indirectly inhibiting, lowering, or altering a pro-inflammatory activity of IL-6, or correcting a hyper-inflammatory or immune dysregulating effect of IL-6 (e.g., dysregulation of differentiation, proliferation, activation, inflammatory cytokine synthesis, and/or apoptotic activity of immune and/or inflammatory effector cells, such as lymphocytes, monocyte/macrophage cells and neutrophils).

Kinase Modulator Drug Combinations with TPA

In other exemplary combinatorial compositions and methods of the invention, an anti-inflammatory effective TPA compound is co-formulated and delivered with, or simultaneously or sequentially co-administered with an kinase modulator drug or agent that directly or indirectly inhibits, lowers, activates or alters an immune or inflammatory activity of one or more kinases involved in mediating or suppressing hyper-inflammatory responses, or regulating differentiation, proliferation, activation, inflammatory cytokine synthesis, and/or apoptotic activity of immune and/or inflammatory effector cells (e.g., lymphocytes, monocyte/macrophage cells and neutrophils). Exemplary target kinases within these aspects of the invention include mitogen activated protein kinase (MAPK) and janus kinase (JAK). Within exemplary embodiments, coordinate treatment of COVID-19 disease subjects with TPA and a JAK inhibitor clinically reduces one or more disease condition(s) or symptom(s) associated with ARDS, SARS, CSS, PIMS, ESHS, DAD or another hyper-inflammatory condition or symptom described herein. In one working example, an anti-inflammatory effective TPA compound is co-formulated or coordinately administered with the JAK inhibitor, Jakotinib hydrochloride, or another JAK inhibitor, baricitinib.

JAK inhibitory anti-COVID-19 treatment strategies herein are based on the role of ACE2 in SARS-COV-2 etiology, discussed in detail above. ACE2 is a cell-surface protein widely expressed in the heart, kidney, blood vessels, and especially pulmonary alveolar epithelia. SARS-COV-2 evidently binds and enter cells through ACE2-mediated endocytosis. One of the known regulators of endocytosis is the AP2-associated protein kinase 1 (AAK1). AAK1 inhibitors can interrupt the passage of the virus into cells and thereby impair viral infection and replication. Baricitinib is both a JAK inhibitor and AAK1 inhibitor, already proven safe in COVID-19 subjects. Therapeutic dosage with either 2 mg or 4 mg baricitinib daily was sufficient to elicit measurable SARS-COV2 inhibition. Concerns about the use of JAK inhibitors are based on reports that JAKs inhibit a variety of cytokines including INF-a, which mediates anti-viral immune responses. There are current clinical trial for the JAK inhibitor jakotinib hydrochloride (“Study for safety and efficacy of Jakotinib hydrochloride tablets in the treatment severe and acute exacerbation patients of novel coronavirus pneumonia (COVID-19)” (ChiCTR2000030170); and “Severe novel coronavirus pneumonia (COVID-19) patients treated with ruxolitinib in combination with mesenchymal stem cells: a prospective, single blind, randomized controlled clinical trial” (ChiCTR2000029580)).

Anti-SARS-COV-2 Vaccine Combinations with TPA

In other exemplary combinatorial compositions and methods of the invention, an anti-viral or anti-inflammatory effective TPA compound is co-formulated and delivered with, or simultaneously or sequentially co-administered with an anti-SARS-COV-2 vaccine agent, to prevent or reduce viral infection and thereby prevent or reduce COVID-19 disease conditions and symptoms. Exemplary candidate vaccines for use within these coordinate treatment methods are currently very numerous, as the entire world scrambles for effective prophylaxis against SARS-COV-2 and other past and potential future hSARS viruses. In certain embodiments, the secondary therapeutic agent in the form of an anti-hSARS viral vaccine may comprise a live-attenuated recombinant hSARS virus or recombinant chimeric hSARS virus, an inactivated or killed hSARS virus, immunogenic subunits of an hSARS virus, for example a subunit vaccine comprised of all or a portion of an hSARS spike(S) protein, among many other diverse anti-hSARS vaccine tools currently being investigated. Vaccines are of particular importance within combinatorial TPA treatment methods and compositions of the invention, in part because their currently remains insufficient evidence that any existing antiviral drug (administered as a monotherapy) will efficiently treat COVID-19 pneumonia.

Vaccine development is a key long-term strategy to prevent COVID-19 renewed outbreaks in the future. With the sequencing of SARS-COV-2 genome, multiple nucleic acid-based vaccine candidates have been proposed, many based on the S protein-coding sequence.

mRNA-1273 Vaccines

In early January of 2020, soon after the outbreak of COVID-19 pneumonia, the genome of SARS-COV-2 was been sequenced. Moderna's mRNA-1273 vaccine candidate is a synthetic strand of mRNA encoding the prefusion-stabilized viral spike(S) protein. After intramuscular injection to human bodies, it is predicted to elicit an antiviral immune response specifically directed toward the spike protein of SARS-COV-2. Unlike conventional vaccines made from inactivated or dead pathogen, live-attenuated virus, or small immunogenic viral subunits, Moderna's lipid nanoparticle-encapsulated mRNA vaccine does not require any use, handling or patient exposure to the SARS-COV-2 virus. Therefore, it is relatively safe and ready to be tested.

If mRNA-1273 proves to be safe for humans and pass the phase I trial, successive evaluation of its efficacy will be carried out immediately (NCT04283461). This vaccine candidate is a prospectively useful secondary agent in combinatorial formulations and coordinate treatment methods with TPA as described herein.

### INO-4800

INO-4800 is a DNA vaccine candidate created by Inovio Pharmaceuticals. Like Moderna's mRNA-1273, INO-4800 is also a genetic vaccine that can be delivered to human cells and translated into proteins to elicit immune responses. Compared to conventional vaccines, genetic vaccines have lower costs of production and are easier and safer to produce and administer. The simple structure of nucleic acids also obviates the risk of incorrect folding, which can occur in recombinant protein-based vaccines. However, the amount of plasmid delivered and the adequate interval and route of administration are uncertain factors that can influence immunogenicity of genetic vaccines. This vaccine candidate is a prospectively useful secondary agent in combinatorial formulations and coordinate treatment methods with TPA as described herein.

ChAdOx1 nCOV-19

The ChAdOx1 nCOV-19 vaccine created by Oxford University is composed of a non-replicating adenovirus vector and the genetic sequence of the S protein of SARS-COV-2, presently in phase I/II clinical trial (NCT04324606). The non-replicating nature of adenovirus in the host makes it relatively safe in children and individuals with underlying diseases. Adenovirus-based vectors are characterized by a broad range of tissue tropism that covers both respiratory and gastrointestinal epithelium, the two main sites that express the ACE-2 receptor of SARS-COV-2. This vaccine candidate is a prospectively useful secondary agent in combinatorial formulations and coordinate treatment methods with TPA as described herein.

### Stabilized Subunit Vaccines

Enveloped viruses require fusion of the viral membrane with the host cell membrane for infection. This process involves the conformational change of the viral glycoprotein from the pre-fusion form to the post-fusion form. Although the pre-fusion glycoproteins are relatively unstable, they are still able to elicit strong immune responses. The University of Queensland is developing a stabilized subunit vaccine against SARS-COV-2 based on the molecular clamp technology, which would allow recombinant viral proteins to stably remain in their pre-fusion form. Previously applied to influenza virus and Ebola virus, molecular clamp vaccines have proved their capacity to induce production of neutralizing antibodies. They are also reported to be potent after two weeks at 37° C. This vaccine candidate is a prospectively useful secondary agent in combinatorial formulations and coordinate treatment methods with TPA as described herein.

### Nanoparticle-Based Vaccines

Nanoparticle-based vaccines represent an alternative strategy to incorporate and prevent antigens to vaccinate at-risk subjects. Through encapsulation or covalent functionalization, nanoparticles can be conjugated with antigenic epitopes, mimic viruses and provoke antigen-specific lymphocyte proliferation as well as cytokine production. In addition, mucosal vaccination through intranasal or oral spray can not only stimulate immune reactions at the mucosal surface, but also provoke systemic responses. This demonstrates the potential of nanoparticle-based vaccines to protect humans against respiratory viruses that cause systemic symptoms. Novavax, Inc. is producing a nanoparticle-based anti-SARS-COV-2 vaccine using antigens derived from the viral S protein. The protein is stably expressed in the baculovirus system, and the product is anticipated to enter phase I trial this summer. This vaccine candidate is a prospectively useful secondary agent in combinatorial formulations and coordinate treatment methods with TPA as described herein.

### Pathogen-Specific Artificial Antigen-Presenting Cells

Based on the knowledge that antigen-specific T cells are able to eradicate cancer cells as well as viral infections, generating large amounts of T cells with viral antigen specificity may provide enhanced resistance to SARS-COV-2 infection. Efficient methods to produce massive amounts of T cells include appropriate antigen-presenting cells that can activate effector T cells, and the differentiation and proliferation of corresponding effector, cytotoxic T cells. Genetically modified artificial antigen-presenting cells (aAPCs) that express conserved domains of viral structural proteins delivered by lentivirus vector can induce naïve T cells to differentiate and proliferate. Multiple trials are underway evaluating the safety and immunogenicity of aAPCs alone and in combination with antigen-specific cytotoxic T cells (NCT04299724, NCT04276896). aAPCs will provide prospectively useful secondary agents in combinatorial formulations and coordinate treatment methods with TPA as described herein.

### Natural Killer Cells

The highest mortality rate inflicted by COVID-19 is observed among elderly patients, at least in part attributable to weakening of the immune system with age. Approaches aimed at boosting innate anti-viral immune responses against SARS-COV-2 are of great potential. Natural killer (NK) cells constitute an important component of the innate immune system mediating rapid responses to viral infection. Previous studies have shown that pulmonary migration of NK cells and macrophages plays a significant role in the clearance of SARS-COV (Chen et al., 2009). The innate response itself, without assistance from CD8+ T cells and antibodies, is able to control SARS-COV infection, by increasing production of cytokines and chemokines. Whether the addition of NK cells can mediate viral clearance in COVID-19 pneumonia is under phase I trial in China (NCT04280224) estimated to be completed by the end of 2020. Several companies aim to repurpose their anti-cancer NK-based products to treat COVID-19. Among them are the jointly developed product from the Green Cross LabCell from South Korea with Kleo Pharmaceuticals from the U.S. Additionally, the USA-based company Celularity has developed placenta haematopoetic stem cell-derived NK cells, CYNK-001. NK cell boosting methods and compositions will provide prospectively useful secondary agents in combinatorial formulations and coordinate treatment methods with TPA as described herein.

### Recombinant Interferon

Type I interferons are secreted by virus-infected cells. When used alone or in combination with other drugs, they exert a broad-spectrum antiviral effect against HCV, respiratory syncytial virus, SARS-COV, and MERS-COV. Trials are now focusing on their safety and efficacy in treating COVID-19 pneumonia (NCT04293887). Type I interferons will provide prospectively useful secondary agents in combinatorial formulations and coordinate treatment methods with TPA as described herein.

### Mesenchymal Stem Cells

Mesenchymal stem cells (MSCs) have been proven to exert anti-inflammatory functions by decreasing pro-inflammatory cytokines and producing paracrine factors to repair tissues. Preclinical evidence has shown that MSCs are able not only to restore endothelial permeability, but also reduce inflammatory infiltrate. The immunomodulating effects of MSCs have been proven on avian influenza viruses, and their role in treating COVID-19 pneumonia is promising. At present, MSCs from the umbilical cord and dental pulp are being clinically tested in COVID-19 studies (NCT04293692, NCT04269525, NCT04288102, NCT04302519). MSCs will provide prospectively useful secondary agents in combinatorial formulations and coordinate treatment methods with TPA as described herein.

### Intravenous Immunoglobulin

Intravenous immunoglobulin (IVIG) has been widely applied in the field of neurology, dermatology and rheumatology. IVIG exerts diverse, dose-dependent effects on the immune system. At low doses (0.2-0.4 g/kg) IVIG is useful as a replacement therapy for antibody deficiencies. At higher doses (up to 2 g/kg) IVIG exhibits potent immunomodulatory functions, including by suppressing inflammatory cell proliferation, inhibiting phagocytosis, and interfering antibody-dependent cytotoxicity (Jolles et al., 2005). Current trials are evaluating safety and efficacy of IVIG in COVID-19 subjects (NCT04261426). IVIG compositions will provide prospectively useful secondary agents in combinatorial formulations and coordinate treatment methods with TPA as described herein.

### SARS-COV-2-Specific Neutralizing Antibodies

The humoral immune response mediated by antibodies is crucial for preventing viral infections. Development of specific viral surface epitope-targeting neutralizing antibodies is a promising approach to target COVID-19. AbCellera (Canada) and Eli Lilly and Company (USA) are co-developing a functional antibody that may neutralize SARS-COV-2 in infected patients. For this purpose, they screened through more than 5 million immune cells from one of the first U.S. patients who recovered from COVID-19, and identified more than 500 promising anti-SARS-CoV-2 antibody sequences, which are currently undergoing screening to find the most effective ones. This approach has been successfully applied to manufacture specific functional antibodies against the West Nile virus. Vir Biotechnology, Inc., ImmunoPrecise, Mount Sinai Health System, and Harbour BioMed (HBM) are also screening to find monoclonal antibodies that will bind and neutralize SARS-COV-2. These and other SARS-COV-2-specific neutralizing antibodies will provide prospectively useful secondary agents in combinatorial formulations and coordinate treatment methods with TPA as described herein.

### Anti-C5a Monoclonal Antibodies

Complement activation is correlated with acute pulmonary injury, with the cytokine C5a (the bioactive molecule cleaved from C5) being implicated as the key effector for mediating tissue injury. The role of C5a includes recruitment of neutrophils and T-lymphocytes, and increasing pulmonary vascular permeability. Anti-C5a treatment has been shown to reduce lung injury by decreasing vascular leakage and neutrophil influx into the lung interstium and alveolar spaces (Guo et al., 2005). Exemplary anti-C5a drugs for use as secondary agents in combinatorial formulations and coordinate treatment methods with TPA include BDB-1, launched by Beijing Defengrei Biotechnology Co., and IFX-1, produced by Beijing Staidson Biopharma and InflaRx.

### Thalidomide

Recently, thalidomide has re-emerged as an antiangiogenic, anti-inflammatory, and anti-fibrotic drug agent for diverse therapeutic uses. By decreasing the synthesis of TNF-alpha, thalidomide has been employed as a treatment for a variety of hyper-inflammatory diseases, such as Crohns disease and Behcets disease. Thalidomide is effective in treating HIN1-infected mice, by reducing infiltration of inflammatory cells and inhibiting or blocking production of pro-inflammatory cytokines (Zhu et al., 2014). Current studies are ongoing to investigate the immunomodulatory effects of thalidomide for lessening lung injury caused by excessive immune/inflammatory responses to SARS-COV-2 (NCT04273529, NCT04273581). Thalidomide is a prospectively useful secondary agent in combinatorial formulations and coordinate treatment methods with TPA as described herein.

### Fingolimod

Fingolimod is an oral immunomodulating agent primarily used to treat refractory multiple sclerosis. It structurally resembles the lipid sphingosine-1-phosphate (SIP), and can act as a highly potent antagonist of SIP1 receptors in lymph node T cells. Through effective binding, S1P1 receptors are internalized and the lymph node T cells are subsequently sequestered Decreased pulmonary influx of T lymphocytes is another approach to attenuate uncontrolled immunopathogenesis currently being studied in a clinical trial (NCT04280588). Fingolimod is a prospectively useful secondary agent in certain combinatorial formulations and coordinate treatment methods with TPA as described herein.

Anti-Angiogenic Drug Combinations with TPA

Elevated levels of vascular endothelial growth factor (VEGF) are observed in patients with acute respiratory distress syndrome. VEGF functions as an inflammatory mediator that can induce endothelial injury and increase vascular permeability (Thickett et al., 2001). A variety of anti-VEGF drugs and biologics have been developed for clinical use. Among these, bevacizumab is a recombinant humanized monoclonal antibody capable of binding and neutralizing VEGF functions, including blocking angiogenesis), now approved and widely used in the US for treating multiple types of cancers. An ongoing trial is now evaluating the effectiveness of Bevacizumab to treat SARS-COV-2 infection (NCT04275414). Anti-angiogenic drug and biologics, including anti-VEGF drugs and biologics, will provide prospectively useful secondary agents in combinatorial formulations and coordinate treatment methods with TPA as described herein.

### Hydroxychloroquine

In other exemplary combinatorial compositions and methods of the invention, an anti-SARS-CoV-2 effective TPA compound is co-formulated and delivered with, or simultaneously or sequentially co-administered with, chloroquine or hydroxychloroquine. Previously long-used as an antimalarial and anti-autoimmune drug, hydroxychloroquine also appears to limit viral infectivity by increasing endosomal pH required for membrane fusion between virus and host cells. In one study hydroxychloroquine is reported to specifically inhibit replication of SARS-CoV by interfering with glycosylation of its cellular receptor, ACE2. Recent in vitro studies suggests that hydroxychloroquine effectively reduces viral load/titer of SARS-COV-2 in COVID-19 subjects (Lan et al., 2020). A number of clinical trials were quickly conducted in China, which reported that hydroxychloroquine was to various degrees effective in treating COVID-19-associated pneumonia. In a small open-label non-randomized clinical trial from France, hydroxychloroquine was reported to have positive effects in combination with azithromycin. The U.S. FDA issued an Emergency Use Authorization for hydroxychloroquine to treat COVID-19 in the USA, and the drug has been widely used domestically off-label for this purpose since. More recent studies have found limited evidence of substantial clinical benefit of hydroxychloroquine for treating COVID-19 disease, and cardiac safety concerns for this drug have led to early closure of at least one major clinical trial.

### Glucocorticoids

Numerous clinical studies have reported the efficacy of glucocorticoids for treating coronavirus and influenza pneumonia. During the SARS epidemic in 2003, glucocorticoid was the main medication of immunomodulatory therapy. Timely usage of glucocorticoid could improve the early fever and reduce the severity of pneumonia and associated hypoxemia. However, some studies did not find beneficial effects with glucocorticoid, and some reports have been made of immunosuppression, delayed viral clearance and adverse reactions. According to international guidelines for management of sepsis and septic shock, if glucocorticoid is to be used, small dosage and short-term application should be applied only for patients in whom adequate fluids and vasopressor therapy do not restore hemodynamic stability.

Systemic glucocorticoids administration has been used for severe complications to suppress CSS manifestations in patients with COVID-19, including ARDS, acute heart injury, acute kidney complication, and patients with elevated D-dimer levels associated with thrombogenesis. Absent further evidence, the interim guideline of WHO does not support the use of systemic corticosteroids for treating viral pneumonia and ARDS associated with COVID-19 disease. Whereas systemic glucocorticoids are currently of questionable use in SARS-COV-2 treatment (based on their potential immunosuppressive effects, prolonging viral clearance), there are nonetheless prospective therapeutic uses for these drugs in treating certain aspects of COVID-19 disease. The underlying pathogenesis of COVID-19 pneumonia is composed of both direct damage caused by the virus and substantial pathogenic impacts caused by hyper-immune and hyper-inflammatory responses of the host. Methylprednisolone administration is contemplated as an exemplary glucocorticoid to help suppress excessive immune and inflammatory reactions, and studies are ongoing to explore its effectiveness and safety in COVID-19 subjects (NCT04273321, NCT04263402). In certain COVID-19 and ARDS treatment contexts, and in targeted and staged treatment protocols, glucocorticoids will provide prospectively useful secondary agents in combinatorial formulations and coordinate treatment methods with TPA as described herein.

### Pharmaceutical Compositions, Dosing, Delivery and Formulation

With respect to each of the foregoing illustrative aspects of the invention, in certain embodiments one or a plurality of TPA or TPA-like compounds will be employed, for example a parent TPA compound of Formula I or Formula II above, such as 12-O-tetradecanoylphorbol-13-acetate (formally “TPA”; also known as phorbol-12-myristate-13-acetate (PMA)), or a structurally related, functional analog, conjugate, prodrug, salt or other modified or derivative form of the parent TPA compound. TPA compounds employed within the invention are useful in compositions and methods administered to subjects to mediate anti-viral (e.g., anti-SARS-CoV-2), anti-anti-inflammatory, anti-ARDs, anti-CSS, anti-PIMS, anti-ESHS, anti-DAD, anti-cytopathic, pro-immune and/or pro-apoptotic effects, among other clinically-relevant activities. The potent and diverse clinical effects of these compositions and methods are individually and collectively effective to treat and/or prevent a diverse range of ARDS and COVID-19 disease conditions, symptoms and attendant immunological, cellular, tissue and organ injuries and dysfunctions.

Generally, a clinically effective amount or dose(s) of a TPA compound of Formula I or II is administered to subjects amenable to TPA treatment to effectively elicit an anti-viral, anti-anti-inflammatory, anti-ARDs, anti-CSS, anti-PIMS, anti-ESHS, anti-DAD, anti-cytopathic, pro-immune and/or pro-apoptotic response in the treated subject. As described above, clinical efficacy is demonstrated by comparing therapeutic indices pre- and post-treatment in test and placebo-treated control subjects. In illustrative embodiments, effective amounts of active TPA compounds will yield quantitatively or qualitatively significant, therapeutic benefits in single or multiple unit dosage form, with a dosing frequency and over a selected period of therapeutic intervention, to alleviate one or more symptom(s) of viral infection, hyper-inflammation, ARDs, CSS, PIMS, ESHS, DAD, elevated cytopathic activity, immunosuppression, dysfunction of normal apoptotic activity of immune and/or inflammatory cells, and/or any other target symptom(s), condition(s) or index (ices) described herein, in treated subjects. Compositions of the invention typically comprise an effective amount or unit dosage of a TPA compound of Formula I or II formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Effective dosing will be readily determined by the clinician, depending on targeted conditions and clinical and patient-specific factors. Suitable effective unit dosage amounts of active TPA compounds for administration to mammalian subjects, including humans, may range from about 10 to about 1500 μg, about 20 to about 1000 μg, about 25 to about 750 μg, about 50 to about 500 μg, about 150 to about 500 μg, about 125 μg to about 500 μg, about 180 to about 500 μg, about 190 to about 500 μg, about 220 to about 500 μg, about 240 to about 500 μg, about 260 to about 500 μg, about 290 to about 500 μg. In certain embodiments, the disease treating effective dosage of a phorbol ester compound or related or derivative compound of Formula I may be selected within narrower ranges of, for example, 10 to 25 μg, 30-50 μg, 75 to 100 μg, 100 to 300 μg, or 150 to 500 μg. These and other effective unit dosage amounts may be administered in a single dose, or in the form of multiple daily, weekly or monthly doses, for example in a dosing regimen comprising from 1 to 5, or 2 to 3, doses administered per day, per week, or per month. In one exemplary embodiment, dosages of 10 to 30 μg, 30 to 50 μg, 50 to 100 μg, 100 to 300 μg, or 300 to 500 μg, are administered one, two, three, four, or five times per day. In more detailed embodiments, dosages of 50-100 μg, 100-300 μg, 300-400 μg, or 400-600 μg are administered once or twice daily. In a further embodiment, dosages of 50-100 μg, 100-300 μg, 300-400 μg, or 400-600 μg are administered every other day. In alternate embodiments, dosages are calculated based on body weight, and may be administered, for example, in amounts from about 0.5 μg/m² to about 300 μg/m² per day, about 1 μg/m² to about 200 μg/m², about 1 μg/m² to about 187.5 μg/m² per day, about 1 μg/m² per day to about 175 μg/m² per day, about 1 μg/m² per day to about 157 μg/m² per day about 1 μg/m² to about 125 μg/m² per day, about 1 μg/m² to about 75 μg/m² per day, 1 μg/m² to about 50 μg/m² per day, 2 μg/m² to about 50 μg/m² per day, 2 μg/m² to about 30 μg/m² per day or 3 μg/m² to about 30 μg/m² per day. In other embodiments, dosages may be administered less frequently, for example, 0.5 μg/m² to about 300 μg/m² every other day, about 1 μg/m² to about 200 μg/m², about 1 μg/m² to about 187.5 μg/m² every other day, about 1 μg/m² to about 175 μg/m² every other day, about 1 μg/m² per day to about 157 μg/m² every other day about 1 μg/m² to about 125 μg/m² every other day, about 1 μg/m² to about 75 μg/m² every other day, 1 μg/m² to about 50 μg/m² every other day, 2 μg/m² to about 50 μg/m² every other day, 2 μg/m² to about 30 μg/m² per day or 3 μg/m² to about 30 μg/m² per day. In additional embodiments, dosages may be administered 3 times/week, 4 times/week, 5 times/week, only on weekdays, only in concert with other treatment regimens, on consecutive days, or in any appropriate dosage regimen depending on clinical and patient-specific factors.

The amount, timing and mode of delivery of therapeutic compositions of the invention will be routinely adjusted on an individual basis, depending on such factors as weight, age, gender, and condition of the individual, the acuteness and severity of the targeted disease or condition, whether the administration is prophylactic or therapeutic, and on the basis of other factors known to effect drug delivery, absorption, pharmacokinetics and efficacy. Effective dosage and administration protocols will often include repeated dosing over a course of several days, one or more weeks, months or even years. An effective treatment regime may also involve prophylactic dosage administered on a day or multi-dose per day basis lasting over the course of days, weeks, months or years.

The pharmaceutical compositions of the invention may be administered by any clinically-acceptable route and means that achieve the intended therapeutic or prophylactic purpose. Suitable routes of administration include all effective conventional delivery routes, devices and methods. Currently practiced delivery methods include injectable methods such as intravenous injection and infusion, intramuscular, intraperitoneal, intraspinal, intrathecal, intracerebroventricular, intraarterial, and subcutaneous injection. Also contemplated are oral and mucosal solid and liquid dosage forms, and intranasal and intrapulmonary aerosol delivery. Effective dosage forms of the invention will often include excipients recognized in the art of pharmaceutical compounding as suitable for the preparation of dosage units. Such excipients include, without limitation, binders, fillers, lubricants, emulsifiers, suspending agents, sweeteners, flavorings, preservatives, buffers, wetting agents, disintegrants, effervescent agents and other conventional excipients and additives. The therapeutic compositions of the invention can further be administered in a sustained, delayed or other controlled release form, for example by use of a slow release carrier or excipient, or a slow, delayed or controlled release polymer. Certain TPA compositions of the invention are beneficially delivered by parenteral administration, e.g. intravenously, intramuscularly, subcutaneously or intraperitoneally. These dosage forms will typically be provided in the form of aqueous or non-aqueous sterile injectable solutions, optionally containing additives like anti-oxidants, buffers, bacteriostats and/or solutes which render the formulation isotonic with the blood of mammalian subjects. Aqueous and non-aqueous sterile suspensions may include suspending agents and/or thickening agents. The formulations may be presented in unit-dose or multi-dose containers. Additional compositions and formulations of the invention may include polymers, liposomes, micelles, conjugates and other agents for improving bioavailability at a specific target (e.g., the lungs) and/or extending release following parenteral administration. Useful parenteral preparations may be solutions, dispersions or emulsions suitable for such administration. Extemporaneous injection and infusion solutions, emulsions and suspensions may be prepared from sterile powders, granules and tablets or other starting forms, according to conventional practices. Useful unit dosage forms will a daily dose or unit, a daily sub-dose, or a therapeutic dose that is effective over a period of multiple days.

In certain embodiments, compositions of the invention may comprise a TPA compound of Formula I or II encapsulated for delivery in microcapsules, microparticles, or microspheres, prepared, for example, by coacervation techniques or by interfacial polymerization, for example, hydroxy methylcellulose or gelatin microcapsules and poly(methylmethacylate) microcapsules, respectively, in colloidal drug delivery systems (for example, liposomes, albumin microspheres, microemulsions, nano-particles and nanocapsules), or within macroemulsions.

Microencapsulation processes are well known and can be routinely implemented to prepare micro particles containing active ampakines described herein.

As noted above, in certain embodiments the methods and compositions of the invention employ pharmaceutically acceptable salts to enhance solubility, bioavailability or other performance criteria, e.g., acid addition or base salts of the above-described TPA compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non-toxic salts, for example aluminum, calcium, lithium, magnesium, potassium, sodium, zinc and diethanolamine salts.

In other embodiments, the methods and compositions of the invention employ prodrugs of phorbol esters of Formula I or II. Prodrugs are considered to be any covalently bonded carriers which release the active parent drug in vivo. Examples of prodrugs useful within the invention include esters or amides with hydroxyalkyl or aminoalkyl as a substituent, and these may be prepared by reacting such compounds as described above with anhydrides such as succinic anhydride. Related aspects of the invention will also be understood to encompass methods and compositions comprising phorbol esters of Formula I or II using in vivo metabolic products of the said compounds (either generated in vivo after administration of the subject precursor compound, or directly administered in the form of the metabolic product itself). Such products may result for example from the oxidation, reduction, hydrolysis, amidation, esterification and the like of the precursor drug or administered compound, primarily due to enzymatic processes. Accordingly, the invention includes methods and compositions for making and using TPA compounds, derivatives and metabolites produced by a process comprising contacting a phorbol ester compound of Formula I or II with a mammalian body fluid, cell, tissue sample or subject for a period of time sufficient to yield a metabolic product of a TPA compound (e.g., starting with a radiolabeled compound administered parenterally in a detectable dose to a mammal, allowing sufficient time for metabolism to occur and isolating a conversion product of the labeled compound from the urine, blood or other biological sample).

The invention disclosed herein will also be understood to encompass diagnostic compositions for diagnosing the risk level, presence, severity, or treatment indicia of, or otherwise managing diseases including, but not limited to, viral infection, hyper-inflammation, ARDs, CSS, PIMS, ESHS, DAD, elevated cytopathic activity, immunosuppression, dysfunction of normal apoptotic activity of immune and/or inflammatory cells, and/or any other target symptom(s), condition(s) or index (ices) described herein, in a mammalian subject. Exemplary diagnostic methods comprising contacting a labeled (e.g., isotopically labeled, fluorescent labeled or otherwise labeled to permit detection of the labeled compound using conventional methods) TPA compound of Formula I or II to a mammalian subject (e.g., to a cell, tissue, plasma, organ, or individual) at risk or presenting with one or more targeted symptom(s), and thereafter detecting the presence, location, metabolism, and/or binding state of the labeled TPA compound using any of a broad array of known assays and labeling/detection methods. In certain embodiments, the TPA compound is isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as 2H, 3H, 13C, 14C, 15N, 18O, 17O, 31P, 32P, 35S, 18F, and 36Cl, respectively. The isotopically-labeled compound is then administered to an individual or other subject and subsequently detected as described above, yielding useful diagnostic and/or therapeutic management data, according to conventional techniques.

As the skilled artisan will understand, this invention is not limited to the particular compounds, formulations, process steps, and materials disclosed herein above, which are provided for illustrative purposes only. Following the discoveries and teachings of the invention as a whole, these compounds, formulations, process steps, and materials can be changed, expanded or substituted in equivalent form and purpose, without undue experimentation. Likewise, the terminology employed herein is exemplary only, to describe illustrative embodiments, and is not intended to limit the scope of the present invention. The following examples are provided for the same, illustrative and non-limiting purpose.

### Example I

### TPA Compounds are Effective for Preventing and Treating Acute Respiratory Distress Syndrome (ARDS), Including Sudden Acute Respiratory Syndrome (SARS) Attending Severe Covid-19 Viral Disease

The inventors have described in detail the discrete cellular, molecular, gene-regulatory, biochemical, physiological and pathogenic effectors, mechanisms, targets, pathways and responses affected and effected by active TPA compounds of the invention, for mediating novel and profound anti-viral, immune-regulatory and anti-hyper-inflammation clinical benefits in patients suffering from ARDS, COVID-19 disease, and other hyper-inflammatory and immune-dysregulatory conditions detailed above. To attempt to exemplify all the targets and activities of TPA within this broad scope of clinical utility would exceed the artist's practical needs and expectations. For economy and clarity of description, the inventors have incorporated by reference all manner and form of cellular, molecular, gene-regulatory, biochemical, physiological and pathologic research and clinical diagnostic assay technologies, tools and methods relating to practical implementation of the full range of activities and clinical mechanisms of TPA compounds and methods disclosed herein.

Certain embodiments of the invention relating to ARDS exemplify a broad range of activities and clinical benefits of TPA compounds generally. In the particular case of ARDS caused by the human SARS coronavirus, SARS-COV-2 (COVID-19), related compositions and uses of TPA for treatment and prevention of ARDS employ and elicit a wide breath of such activities and benefits. For example, our pilot studies to investigate clinical optimization of TPS compounds and methods for treatment of ARDS clarify a host of critical pro-immune and anti-inflammatory targets, mechanisms and pathway whereby TPA compounds target and eliminate or reduce aberrant effector cells, molecular and biochemical effectors and downstream impacts that cause immune dysfunction and hyper-inflammation, while in other aspects TPA compounds and methods of the invention are found to interact with and induce or promote beneficial immune-regulatory cells and their associated molecular and biochemical effectors, thus beneficially modifying their downstream impacts. In like manner TPA compounds inhibit, prevent, promote and/or modify other immune and inflammatory targets known to mediate related immune dysfunction and hyper-inflammatory conditions, such as Cytokine Storm Syndrome (CSS), Pediatric Inflammatory Multisystem Syndrome (PIMS), Extrapulmonary Systemic Hyperinflammation Syndrome (ESHS), and vascular congestive and thrombotic conditions attending severe COVID-19-associated ARDS (e.g., Disseminated Intravascular Coagulation (DIC), thrombosis, stroke, thrombocytopenia, and gangrene, among other cellular, tissue and organ injuries that attend these conditions).

The instant disclosure, in conjunction with ongoing pilot studies by the inventors, illustrates a wide range of pro-immune and anti-inflammatory effects of TPA that will potently mediate reduction and/or prevention of ARDS conditions and symptoms, as well as other pathogenic conditions and symptoms associated with severe COVID-19 disease. In this context TPA compounds have multiple demonstrated pro-immune and anti-inflammatory activities, mediated through key immune- and inflammatory-regulating cells, gene targets and molecular and biochemical targets such as cytokines, chemokines and kinases. Exemplary pilot studies have shown that TPA compounds will effectively treat and prevent lymphocytopenia, by virtue that these compounds promote full range of pro-immune effects relating to activation, proliferation and survival of lymphocytes. According to the further teachings, TPA compounds administered to SARS-COV-2 subjects presenting with ARDS clinically supports normal health, function and survival of lymphocytes (including T and B cells, and NK cells known to be critically impaired and numerically reduced in severe COVID-19/ARDS cases). In related aspects, TPA has been shown to exert pro-apoptotic effects, through interactions with immune-regulatory kinases, such as MAPK and JAK, that will potently correct aberrant programming and activation of neutrophils implicated as key effectors in the etiology or ARDS, CSS, PIMS, ESHS and other conditions associated with severe COVID-19 disease. Neutrophils are evidently “hijacked” and reprogrammed by SARS-COV-2 proteins (and other emergent pro-inflammatory factors that emerge in the course of COVID-19 disease development), leading to profound dysregulation of neutrophil immune and inflammatory activities. This includes a range of hyper-inflammatory activities of neutrophils and their upstream regulatory cells and signals, including: hyper-elevation of pro-inflammatory cytokines and chemokines; increased neutrophil and macrophage migration (extravasation) into pulmonary and alveolar compartments (aided by breakdown of vascular and endothelial barriers); hyper-elevation of pathogenic neutrophil and macrophage activities (including tissue- and extracellular matrix-destructive degranulation, increasing oxidative stress of cells and tissues (e.g., through hyper-liberation of peroxides and other ROS), hyper-phagocytosis, and deposition of neutrophil extracellular traps (NETs) associated with alveolar and vascular congestion and thrombogenesis); and dysregulation of normal neutrophil and macrophage apoptosis (by disrupting spontaneous apoptosis, and inhibiting phagocytosis-induced cell death (PICD), likely mediated in part by the SARS-COV-2 N protein), which aberrantly prolongs neutrophil and macrophage lifespan and extends and otherwise dysregulates their inflammatory activities, contributing profoundly to COVID-19 tissue and organ pathogenesis.

Yet additional activities of TPA established through our pilot investigations evince profound activities of TPA for blocking and reducing hyper-elevated pro-inflammatory cytokine activation critically associated with CSS and ARDS attending severe COVID-19 disease. TPA compounds block or impair hyper-induction, hyper-synthesis and hyper-activity of pro-inflammatory cytokines directly implicated in CSS and ARDS through a variety of targets, mechanisms and pathways as described. Through these activities, our evidence shows that anti-inflammatory TPA compounds and methods of the invention will additionally block and impair such hyper-inflammatory sequelae of SARS-COV-2 infection as; lymphocytopenia (e.g., by blocking or inhibiting pro-inflammatory hyper-activation of T, B and NK lymphocytes, thereby reducing activation-induced lymphocytic cell death), and hyper-infiltration, hyper-activation and elevated numbers of destructive macrophages and neutrophils in the lung parenchyma, pulmonary alveolar airspaces, and other CSS-affected tissues and organs. These anti-inflammatory effects of TPA that limit pro-inflammatory cytokine expression and activity will in turn mediate reduction of COVID-19/CSS/ARDS-associated pathogenic conditions, including: oxidative stress; endothelial and epithelial barrier destruction; fibrosis and other inflammatory injuries to the lungs and other CSS-affected tissues and organs; inflammatory injury, loss and atrophy of lymph nodes; inflammatory injury and atrophy of the spleen; sepsis; Toxic Shock Syndrome (TSS), and other pathologies.

Toward our purpose of descriptive economy, the inventors have provided representative scientific examples, explanations and analyses throughout this specification, together with citations directing the reader to publications that provide additional technical description of known, available research and diagnostic assay tools and methods (including a full range of in vitro cell-based, biochemical, molecular and gene-regulatory assays, as well as in vivo investigative and diagnostic clinical assays). These research assay and clinical diagnostic tools and methods follow the detailed description and cross-referenced citations to supportive learned articles herein. Each of the publications cited herein is incorporated for all purposes, including to supplement this description with technical materials and methods aimed at research and clinical protocols and objectives known and readily practiced in the art.

To further clarify understanding and practice of the invention herein, the inventors are presently extending their pilot studies toward pre-clinical and clinical trials in animal and human subjects, focusing on clinical use of anti-viral and anti-inflammatory TPA compounds to reduce or prevent ARDS and CSS associated with SARS-COV-2-infection.

### Preclinical Studies of TPA Compounds for Treating and Preventing ARDS

The efficacy of TPA compounds and methods of the invention for mediating multiple, broad pro-immune and anti-inflammatory effects to treat and prevent ARDS will be further evinced by studies employing the well-known endotoxin-induced murine model of ARDS. Employing this model, it will be shown that TPA compounds diminish pulmonary histopathologic changes (including extravasation of neutrophils, thromboses marked by red blood cells extravasated and coagulated in lung parenchyma and alveolar airspaces, and thickening of the alveolar walls. TPA compounds will also inhibit endotoxin-induced increases in protein content found in bronchoalveolar lavage (BALF) samples of study subjects, confirming a protective function of TPA against destruction of endothelial and epithelial barriers. Endotoxin-induced release of pro-inflammatory cytokines will also be reduced in study subjects treated with TPA, for example as confirmed by documented endotoxin-induced hyper-stimulation of tumor necrosis factor-alpha (TNFα). Within these studies lavaged neutrophils from TPA-treated subjects will also show to generate lower levels of reactive oxygen species (ROS) further evincing anti-inflammatory, pro-immune (including protection of lymphocytes) and barrier-protective effects of TPA compounds mediated through neutrophil-regulatory and other immune-effecting signal-cascade targets and pathways described herein. Related assays following the extensive references provided herein will confirm protective effects of TPA compounds on the ability of in vitro endothelial monolayers to resist peroxide-induced barrier dysfunction. These data will confirm that TPA broadly regulates pro-immune functions and inhibits or “re-programs” hyper-inflammatory functions, particularly via direct effects on immune and inflammatory effector cells (T, B and NK lymphocytes, monocyte/macrophage cells, and neutrophils) implicated as key players in severe COVID-19/ARDS pathogenesis, and on the upstream signals (cytokines and chemokines) and molecular/genetic regulatory and “switch” effectors (such as MAPK and JAK kinases) that program and drive their differentiation, activation, cytokine and receptor synthesis/response activities, migration and longevity/apoptosis.

In the mouse model of endotoxin-induced ARDS, a TPA iv composition is injected in the mouse tail vein 4 h after endotoxin instillation into the lungs. At this time point, mice exhibit hypothermic shock, and the lungs already show signs of neutrophil infiltration (inflammation). Thus, this model demonstrates the effects of TPA on ongoing ARDS pathogenic development. At both 24 and 48 h post-injection, histopathologic changes of the lung will be markedly suppressed in mice receiving TPA compound. At 48 hrs the pro-immune and anti-inflammatory effects of TPA will be further demonstrated by reduction of protein extravasation in BALF samples, including reduction of TNFα content in these samples. Lavaged neutrophils from the bronchioalveolar compartment of study mice receiving TPA will show significant reduction in ROS generation after 48 hours, and endothelial monolayers pre-incubated with TPA will likewise exhibit increased resistance to peroxide-induced barrier dysfunction.

Study design For assessment of TPA effects on endotoxin-induced lung injury, LPS endotoxin or saline is delivered into the lungs of isoflurane-anesthetized 20-25 g C57Bl/6 mouse as previously described (Zhang et al., 2013). A nested range of multiple TPA dosage is selected according to the above description for different study groups, in order to assess dose-dependent safety and efficacy, and injected into the tail veins 4 h after LPS administration. Animal temperature is determined every 2 h. 24-48 h later animals are sacrificed for analysis.

For assessment of vascular leak and lung inflammation, Evans Blue Dye (EBD)-albumin conjugate (0.5% EBD in 4% BSA solution) is administered in the tail vein (30 mg/kg) 1 h prior to experiment termination. In anesthetized animals the chest cavity is opened and blood is sampled by cardiac puncture to determine levels of circulating EBD. Lungs are washed for gross pathological exam, histopathology and collection of lavage samples.

This study design allows tracking of TPA efficacy when therapeutic intervention is initiated upon the first clinical signs of ARDS. To further correlate study points with human ARDS status and response, animal temperature and neutrophil infiltration in BALF are closely monitored. The mice experience hypothermic response peaking at 4-6 h following LPS administration. Total cell number of white blood cells (WBCs) and neutrophils in BALF are measured after administering saline or LPS. Within 4 h post-LPS administration, the presence of neutrophils in BALF from TPA-treated subjects will already be significantly reduced in comparison to saline-injected control subjects.

To further evaluate ARDS pathogenesis in this model, hematoxylin and eosin staining of lung sections will also demonstrate TPA-inhibition of hyper-inflammatory lung pathogenesis. As LPS-triggered lung inflammation progresses to a more severe stage at 48 h, TPA treated subjects will show marked reduction in neutrophil and RBC into pulmonary interstitial spaces and alveolar airspaces, as well as reduced swelling of the alveolar walls.

TPA effects on lung permeability and neutrophil infiltration will also correlate with reduced levels of extravasated proteins and cytokines in treated subjects, as indicated by TPA suppression of LPS-induced EBD extravasation, as well as with a reduction in monocyte/macrophage and neutrophil counts in BALF and in histological samples of the lung interstitium and alveolar airspaces. The balance between macrophages, lymphocytes and neutrophils in BALF TPA-treated mice will also show significant protective activity of TPA on lymphocytes (to ameliorate lymphocytopenia observed in ARDS), and suppression of hyper-stimulation and pro-apoptotic regulation of macrophage and neutrophil populations (whereby the total number and ratio of these cells to WBC's will be reduced).

To further characterize TPA efficacy against ARDS, the ability of BALF WBCs from TPA and control mice to generate ROS is measured. WBC ROS production will be significantly lower in mice receiving TPA.

This model further provides for elucidation of TPA effects on pro-inflammatory, pro-immune, anti-inflammatory and other cytokine effects associated with ARDS disease development and protection. Endotoxin-induced ARDS subjects in this model exhibit profound hyper-inflammatory increases in pro-inflammatory cytokines, including IL-10. TPA will block or reduce hyper-elevation of IL-6, IL-10, TNFα, MIP2 and other pro-inflammatory cytokines measured in BALF and histological samples from these subjects.

To evaluate TPA protective effects on endothelial and epithelial barrier integrity, and protection against oxidative stress, endothelial and epithelial monolayer barrier function is assessed directly. In one protocol, human pulmonary artery endothelial cells (HPAECs) are grown in monolayers on collagenized polyester inserts in the presence of TPA in the lower chamber. Prior to analysis, inserts are transferred to fresh wells to avoid direct ROS scavenging. The top chamber is loaded with FITC-dextran, and monolayers are stimulated with edemagenic product of the neutrophil oxidative burst 250 μM H202. Marked HPAEC barrier dysfunction (evinced by transendothelial electrical resistance (TER) studies) is observed in control subjects in response to H202, which response will be significantly attenuated or blocked by TPA treatment of ASC. The following exemplary protocols and materials are in current implementation to advance illustrative aspects of the invention.

### In-Life Scope-of-Work: TPA/ARDS STUDY IN MICE

### 1. Objective

1.1. To determine the efficacy of the inflammation-regulatory drug 12-O-tetradecanoylphorbol-13-acetate (TPA) to prevent pulmonary hyper-inflammation in a mouse model of Adult Respiratory Distress Syndrome (ARDS)

### 2. Materials and Details

2.1. Test and control articles/cells in ready-to-administer format or stock concentration
2.2. Instructions for thawing and dilution for the cell product: storage and stability information
2.3. A Study Protocol must be approved by the sponsor prior to initiation of the study

### Target Data/Deliverables

3.1. In-Life Draft Report, inclusive of all study data
Individually tabulated and QC'd raw animal observation data, in-life observations, dose-group assignment, body weights, and adverse events
Necropsy findings and macroscopic evaluation

### 3.2 Histopathology Report

| 4. In-Vivo Study Parameters General Overview | In overview, TPA is administered to mice in a | | | | |
|---|---|---|---|---|---|
| (provided by the sponsor) | nested range of dosages intravenously, following | | | | |
| | induction of ARDS in the mice by pulmonary | | | | |
| | instillation of endotoxin (lipopolysaccharide (LPS)). | | | | |
| | The control group receives endotoxin induction | | | | |
| | followed by sham, saline injection. After a brief | | | | |
| | study period of 24-76 hours, the mice are euthanized | | | | |
| | and samples of Bronchoalveolar lavage fluid | | | | |
| | (BALF), blood and tissue are collected for cellular, | | | | |
| | histological and biochemical assays that each | | | | |
| | correlates with ARDS severity. | | | | |
| Test System | Naïve, WT C57BL/6 mice-8-10 weeks old | | | | |
| | N = 50 on study, Female | | | | |
| Acclimation | Source: Jackson Labs | | | | |
| | At least 1 week prior to the study initiation, or per | | | | |
| | ASC's SOP | | | | |
| Duration of Study | Food and Water is offered ad libitum | | | | |
| Treatments | ~4 days (performed in different staggers) | | | | |
| | Phase 1: ARDS Induction | | | | |
| | Endotoxin (LPS) from Escherichia coli 055:B5 (15 | | | | |
| | mg/kg, Sigma-Aldrich, St. Louis, MO, USA) or | | | | |
| | PBS vehicle alone (each) | | | | |
| | Route: Intranasal infusion or pipetted into the back | | | | |
| | of the throat | | | | |
| | ASC will purchase LPS (assumes simple | | | | |
| | formulation in PBS) | | | | |
| | Phase 2: TPA Treatments | | | | |
| | 1-4 hours (to be determined) after LPS | | | | |
| | administration, the TPA or PBS will be | | | | |
| | administered via tail vein injections (i.v.) | | | | |
| Experimental | I. Phase 1: ARDS Induction | | | | |
| Design | To induce ARDS, female C57BL/6 mice aged 8 to 10 weeks are anesthetized by | | | | |
| | inhalation of 2% isoflurane vapor mixed with oxygen. | | | | |
| | Anesthetized mice are suspended by their cranial incisors, and the tongue is | | | | |
| | extracted to full extension to prevent the swallowing reflex. | | | | |
| | 50-75 μl of LPS from Escherichia coli 055:B5 (15 mg/kg, Sigma-Aldrich, St. | | | | |
| | Louis, MO, USA) or PBS vehicle alone (each) is pipetted into the back of the | | | | |
| | throat, and the nares are pinched shut to force breathing through the mouth and | | | | |
| | aspiration of the liquid. | | | | |
| | Up to N = 50 mice (40 on study plus 10 extra as replacement) will be used | | | | |
| | forLPS-induced ARDS as outlined below: | | | | |
| | Induction will be performed in multiple staggers (exact stagger information e.g. | | | | |
| | no. of mice injected, will be detailed in the study protocol) | | | | |
| | N | Treatment & Dose | Route | End Points | |
| | 50 | LPS | Intratracheal, | Select N = 40 mice in total for | |
| | | 15 mg/kg in PBS | Or pipette into | Phase 2, TPA Treatment | |
| | | | the back of the | | |
| | | | throat | | |
| | Phase 2: Main Study, Test Article (TPA) Treatments | | | | |
| | ~2 h post LPS administration, the test article (TPA) or PBS will be administered | | | | |
| | via tail vein injections | | | | |
| | Animals will be sacrificed at 48 h and 72 h post-LPS exposure by exsanguination | | | | |
| | under anesthesia, terminal blood samples will be collected via cardiac puncture | | | | |
| | The lungs are processed for Bronchoalveolar lavage (BAL) collection, RNA or | | | | |
| | protein isolation, or histology | | | | |
| | Group Assignment Table 2: | | | | |
| | Group | N | Dose | Route | End Points |
| | 1 | 10 | PBS | Single, i.v. | Terminate N = 5 each at: |
| | 2 | 10 | TPA, low | injection | 48 h and 72 h post-LPS administration |
| | 3 | 10 | TPA, mid | 2 h-post | Collect BALF |
| | 4 | 10 | TPA, high | LPS | Terminal Blood |
| | | | | induction | Lungs Samples |
| | | | | | (split in 2 halves, 1 each flash |
| | | | | | frozen, and other in fixative |

| | |
|---|---|
| Cageside | Twice Daily |
| Observations | |
| Clinical | Prior to LPS-administration and pre-sacrifice |
| Observations and | |
| Body Weights | |
| Bronchoalveolar | To collect Bronchoalveolar Lavage Fluid (BALF) samples; |
| Lavage | II. Immediately after exsanguination, the lungs will be cannulated (or intra-tracheal |
| | cannulation) with a 20-gauge intravenous catheter and gently washed five times |
| | sequentially with 1 ml (whole lung) PBS supplemented with 0.4 mM EDTA |
| | (GIBCO) and protease inhibitor cocktail (Roche, Indianapolis, IN, USA) |
| | III. Cells from all five lavage collections are stored for cell counting (see below) while |
| | the lavage supernatant is stored at −80° C. for biochemical analysis. |
| Necropsy and | At the time of sacrifice (48 h and 72 h post-LPS), lung samples will be collected through the |
| Tissue Collection | trachea and split into 2 halves (right and left) |
| | III. One half will be placed into a specified fixative (10% neutral buffered formalin) and |
| | stored at 4° C. until histopathology analysis |
| | IV. Second half will be flash frozen separately, in 2 parts and stored until shipment to |
| | the sponsor's designated lab for downstream inflammatory marker expression |
| | analysis at protein and RNA level (PCR) |
| General | Macroscopic findings will be recorded for general pulmonary anatomy at the time of |
| Pulmonary | sacrifice |
| Anatomy and | Lung samples will be processed and H&E stained, and microscopically evaluated |
| Histology | for pulmonary histology, including immunoinfiltration, vascular changes, septal |
| | thickening, etc. among test and control samples |
| | Histology will be performed at ASC-partner lab (StageBio) |
| | The histology lab will receive the lungs from 40 mice in fixative. All study-related |
| | documentation (e.g., gross findings, protocol, protocol amendments, etc.) will also |
| | be provided. |
| | The lungs from all animals will be trimmed, processed, embedded in paraffin, and |
| | microtomed. All tissues will be stained with H&E and coverslipped. Slides will be |
| | microscopically quality checked. |
| | An ACVP Veterinary Pathologist will microscopically evaluate all H&E stained |
| | sections. |
| | The draft pathology report, consisting of tabulated microscopic data and a |
| | discussion of noteworthy changes, requires ~4-5 weeks for completion from receipt |
| | tissues and documentation. Actual timeline will be developed based on available |
| | histology and pathology resources at the time of arrival of tissues and associated |
| | study documentation. |
| | Photomicrographs will be taken and annotated for inclusion as an appendix of the |
| | pathology report. Up to 10 images, as needed to depict representative microscopic |
| | changes, will be included. Charges will apply only for actual number of images |
| | required to meet the request. |

| Experimental | I. Phase 1: ARDS Induction | | | | |
|---|---|---|---|---|---|
| Design | To induce ARDS, female C57BL/6 mice aged 8 to 10 weeks are anesthetized by | | | | |
| | inhalation of 2% isoflurane vapor mixed with oxygen. | | | | |
| | Anesthetized mice are suspended by their cranial incisors, and the tongue is | | | | |
| | extracted to full extension to prevent the swallowing reflex. | | | | |
| | 50-75 μl of LPS from Escherichia coli 055:B5 (15 mg/kg, Sigma-Aldrich, St. | | | | |
| | Louis, MO, USA) or PBS vehicle alone (each) is pipetted into the back of the | | | | |
| | throat, and the nares are pinched shut to force breathing through the mouth and | | | | |
| | aspiration of the liquid. | | | | |
| | Up to N = 50 mice (40 on study plus 10 extra as replacement) will be used | | | | |
| | forLPS-induced ARDS as outlined below: | | | | |
| | Induction will be performed in multiple staggers (exact stagger information e.g. | | | | |
| | no. of mice injected, will be detailed in the study protocol) | | | | |
| | N Treatment & Dose Route End Points | | | | |
| | Intratracheal, LPS Or pipette into Select N = 40 mice in total for15 | | | | |
| | 50 mg/kg in PBS the back of the throat Phase 2, TPA Treatment | | | | |
| | Phase 2: Main Study, Test Article (TPA) Treatments | | | | |
| | ~2 h post LPS administration, the test article (TPA) or PBS will be administered | | | | |
| | via tail vein injections | | | | |
| | Animals will be sacrificed at 48 h and 72 h post-LPS exposure by exsanguination | | | | |
| | under anesthesia, terminal blood samples will be collected via cardiac puncture | | | | |
| | The lungs are processed for Bronchoalveolar lavage (BAL) collection, RNA or | | | | |
| | protein isolation, or histology | | | | |
| | Group Assignment Table 2: | | | | |
| | Group | N | Dose | Route | End Points |
| | 1 | 10 | PBS | Single, i.v. | Terminate N = 5 each at: |
| | 2 | 10 | TPA, low | injection 2 h- | 48 h and 72 h post-LPS administration |
| | 3 | 10 | TPA, mid | post LPS | Collect BALF |
| | 4 | 10 | TPA, high | induction | Terminal Blood |
| | | | | | Lungs Samples |
| | | | | | (split in 2 halves, 1 each flashfrozen, |
| | | | | | and other in fixative |

| | |
|---|---|
| Cageside | Twice Daily |
| Observations | |
| Clinical | Prior to LPS-administration and pre-sacrifice |
| Observations and | |
| Body Weights | |
| Bronchoalveolar | To collect Bronchoalveolar Lavage Fluid (BALF) samples; |
| Lavage | II. Immediately after exsanguination, the lungs will be cannulated (or intra-tracheal |
| | cannulation) with a 20-gauge intravenous catheter and gently washed five times |
| | sequentially with 1 ml (whole lung) PBS supplemented with 0.4 mM EDTA |
| | (GIBCO) and protease inhibitor cocktail (Roche, Indianapolis, IN, USA) |
| | III. Cells from all five lavage collections are stored for cell counting (see below) while |
| | the lavage supernatant is stored at-80° C. for biochemical analysis. |
| Necropsy and | At the time of sacrifice (48 h and 72 h post-LPS), lung samples will be collected through the |
| Tissue Collection | trachea and split into 2 halves (right and left) |
| | III. One half will be placed into a specified fixative (10% neutral buffered formalin) and |
| | stored at 4° C. until histopathology analysis |
| | IV. Second half will be flash frozen separately, in 2 parts and stored until shipment to |
| | the sponsor's designated lab for downstream inflammatory marker expression |
| | analysis at protein and RNA level (PCR) |
| General | Macroscopic findings will be recorded for general pulmonary anatomy at the time of |
| Pulmonary | sacrifice |
| Anatomy and | Lung samples will be processed and H&E stained, and microscopically evaluated |
| Histology | for pulmonary histology, including immunoinfiltration, vascular changes, septal |
| | thickening, etc. among test and control samples |
| | Histology will be performed at ASC-partner lab (StageBio) |
| | The histology lab will receive the lungs from 40 mice in fixative. All study-related |
| | documentation (e.g., gross findings, protocol, protocol amendments, etc.) will also |
| | be provided. |
| | The lungs from all animals will be trimmed, processed, embedded in paraffin, and |
| | microtomed. All tissues will be stained with H&E and coverslipped. Slides will be |
| | microscopically quality checked. |
| | An ACVP Veterinary Pathologist will microscopically evaluate all H&E stained |
| | sections. |
| | The draft pathology report, consisting of tabulated microscopic data and a |
| | discussion of noteworthy changes, requires ~4-5 weeks for completion from receipt |
| | tissues and documentation. Actual timeline will be developed based on available |
| | histology and pathology resources at the time of arrival of tissues and associated |
| | study documentation. |
| | Photomicrographs will be taken and annotated for inclusion as an appendix of the |
| | pathology report. Up to 10 images, as needed to depict representative microscopic |
| | changes, will be included. Charges will apply only for actual number of images |
| | required to meet the request. |

Demonstration that TPA Treatment Regulates and Attenuates Immune/Inflammatory Responses Including by Biasing these Responses Toward Th1 Versus Th2 Activation

TPA will minimize potential for severe Covid-19 disease by regulating and attenuating viral-induced hyper-immune and hyper inflammatory activation, including CSS (by blocking or reducing over-expression of pro-inflammatory cytokines associated with hyper-activation of inflammatory signaling and cellular responses). In certain embodiments TPA will mediate attenuated, immune-selective dampening of Th2 T cell responses associated with hyper-immune and hyper-inflammatory activity, and will mediate neutral or potentiating effects on beneficial Th1-biased T helper cell differentiation and marker expression. Various assays will be useful to demonstrate that TPA administration to animal or human subjects will mediate a regulated/attenuated immune response, typically biased toward Th1-versus Th2-specific cytokine/chemokine/growth factor expression patterns (in comparison to the patterns determined among non-treated control subjects). In one illustrative assay, peripheral blood mononuclear cells from ARDS-induced, SARS-COV-2 infected, or hCoV pseudovirus-exposed subjects (including cells and/or tissues), with or without TPA treatment (i.e., treatment before, during or after ARDS induction, or viral or pseudoviral exposure/activation) are analyzed to assess marker expression, histology, histochemistry, and differentiation of immune cells in the TPA-treated versus non-treated control samples. In certain assays, PBMC samples are isolated from animal subjects following ARDS induction (e.g., using endotoxin induction), without virus present, while other useful assays will employ samples from subjects exposed to hCoV-2, or from CoV pseudovirus-exposed animal or human subjects, to evaluate Th1-biased versus Th2-biased T cell differentiation and marker expression. In exemplary studies, cellular immune responses in TPA-treated test subjects and non-treated control model subjects may be evaluated using a multi-color T-cell ELISpot (e.g., as provided by CTL Laboratories). CD4+ Th1 responses are discerned, for example, by measuring IFN-γ, whereas CD4+ Th2 responses can identified by measuring IL-5, among other useful activation/differentiation markers. TPA will mediate a regulated, attenuated immune/inflammatory response that is biased toward Th1 T cell activation/differentiation and features dampened Th2 activation/differentiation compared to controls, lowering the hyper-immune and hyper-inflammatory activation associated with CSS and ARDS.

The enzyme-linked immune absorbent spot (ELISpot) is a highly sensitive and specific assay that quantitatively measures the frequency of cytokine or immunoglobulin secretion by a single cell. ELISpot has been widely applied to investigate specific immune responses in infections, cancer, allergies and autoimmune diseases. With detection levels as low as one cell in 100,000, ELISpot is among the most sensitive cellular assays currently available. The FluoroSpot Assay is a variation of the ELISpot assay, using fluorescence to analyze multiple cytokines in a single well. ELISpot assays are carried out in a 96-well plate, and an automated ELISpot reader is used for analysis. The assay is therefore robust, easy to perform and suitable for large-scale trials. T-cell ELISpot is widely applied in investigations of specific immune responses in infectious diseases, cancer, allergies, and autoimmune diseases. Within the instant invention, T-cell ELISpot assays are particularly useful to guide development and monitor the efficacy and safety of TPA and related compounds to mediate healthy, balanced and attenuated immune and inflammatory responses to SARS-COV-2 and other respiratory viral infection, to lower the risk and occurrence of ARDS and other related conditions as described herein (e.g., pneumonia, vasculitis, thrombogenesis, etc.)

### CSS Assays

Additional assays will elucidate potent activity of TPA for preventing CSS and associated adverse hyper-immune and/or hyper-inflammatory responses, that contribute to ARDS in COVID-19 patients. In view of the complex and uncertain immune and inflammatory interactions attending SARS-COV-2 infection and COVID-19 disease, that have yet to be fully understood, the targets of these assays are fundamental-focusing on the potential for Ii-Key-SARS-COV-2 peptides to induce expression of pro-inflammatory cytokines associated with CSS. Exemplary cytokine assays employ a modified cytometric bead array (CBA) screen, using a flow cytometry system adapted to quantify multiple cytokines simultaneously, for example in cell culture supernatants (SN), or in biological fluids such as serum or plasma. The CBA system uses the broad dynamic range of fluorescence detection offered by flow cytometry, along with antibody-coated beads to efficiently capture analytes. Each bead in the array has a unique fluorescence intensity so that beads capturing different analytes can be mixed and run simultaneously in a single tube. This method significantly reduces sample volumes and time to results in comparison to traditional ELISA and Western blot techniques.

Briefly, target cytokines are captured from lysate, serum or supernatant by capture antibodies conjugated to beads. Detector antibody labeled with fluorochrome binds to various captured cytokines, and the fluorescent signals are recorded during sample acquisition by flow cytometer. The intensity of signal depends on the concentration of each cytokine and can be used to calculate the concentration of specific cytokines using a protein standard curve. Using different size and fluorescence of beads for different capture cytokines, the signals recorded for different cytokines can be distinguished by flow cytometric analysis for multiple analytes in one test sample.

Fluorescent signal provided by each cytokine captured by antibody-coated beads and labeled with detection antibody is defined as mean fluorescence intensity (MFI). This value can be converted into concentration for each cytokine in a test sample using a standard curve generated by measuring MFI from the standards (samples with known concentrations of the given analyte). The increased concentration of cytokine is detected in the cell culture supernatant when the cells secrete cytokines after ARDS induction, or viral or pseudoviral exposure, allowing TPA treatment values to be compared control values (e.g. to demonstrate that TPA substantially reduces activation/expression of multiple pro-inflammatory cytokines otherwise activated or elevated (in control subjects/samples) in response to ARDS induction and/or natural or artificial viral infection.

The utility of these screening assays for demonstrating efficacy and optimal dosing of TPA compounds will allow regular prophylactic and therapeutic treatment to prevent or reduce hyper-immune and hyper-inflammatory responses to SARS-COV-2 and other viral infections in humans. These assays are expected to further evince that TPA will be useful in conjunction with SARS-COV vaccination programs, to reduce risks of vaccine-induced hyper-immune or -inflammatory responses. In particular TPA will be effective to reduce “Antigen Dependent Enhancement” (ADE) in SARS-COV-2 vaccinees. ADE has been shown to contribute to a greater risk of hyper-immune and hyper-inflammatory responsiveness in vaccinees versus non-vaccinated subjects, upon subsequent natural exposure to the target virus of the vaccine. This has been demonstrated for Dengue and other viruses among vaccinees, and ADE is predicted to be a likely complication in for Coronavirus vaccine programs. It has been noted that more severe COVID-19 cases are associated with increased exposure to prior endemic hCoV viral infections (including regular, 2-3 year cyclic infections of adults by any of four endemic hCoVs that cause common colds, which likely cross react with SARS-COV-2 contributing to ADE in older, more exposed adults. The same pre-disposition to hyper-immune and hyper-inflammatory responsivity upon later wild-type viral challenge is likely to follow Covid-19 vaccination, after the vaccine efficacy wanes (following a predicted decline in neutralizing anti-SARS-COV-2 antibodies in vaccinees), and may also attend booster vaccination. TPA treatment will attenuate these aberrant ADE responses, alleviating ADE risk or severity in vaccinees showing diminished immunity who are again at risk of natural SARS-COV-2 infection, and also at the time of booster vaccination.

It is well established that increased, unregulated expression of pro-inflammatory cytokines and chemokines (CSS) mediates excessive damage of organs and tissues in ARDS, including that attending severe COVID-19 disease. CSS is observed often during the acute phase of inflammation and infectious disease. Cytokines that are up-regulated and likely contribute to organ and tissue damage in severe COVID-19 disease patients have been reported to include IL-4, IL-6, IL-8, IL-10, IL-12, IL-15, IL-17, CCL2 (MCP-1), CXCL9 (Mig), and CXCL10 (IP-10), among others. Functional roles of pro-inflammatory cytokines in CSS and tissue/organ damage, for example, include the following:
IL-6, IL-8, CCL2-activation/recruitment of neutrophils and monocytes
IL-17-tissue inflammation in autoimmune diseases (MS, IBD)
CXCL chemokines (e.g., Mig, IP-10)-recruitment of NK and T cells into organs/tissues

For calibrating and clarifying the results of CBA assays for use within the invention (e.g., to elucidate fine tune TPA efficacy and dosage for reducing CSS potential), expression levels for a panel of pro-inflammatory cytokines known to be associated with CSS and ARDS in severe COVID-19 patients are tested and compared between test and control subjects as described (e.g., using ARDS-induced animals, or SARS-COV-2 or pseudovirus-infected animal or human cellular or individual subjects). The panel of pro-inflammatory cytokines tested will include, for example, human IL-6, IL-8, IL-10, IL-17, IFN-γ, TNF, MCP-1 (CCL2) and Mig (CXCL9). Assays are performed according to well known, published methods, often including establishment of a baseline for pro-inflammatory cytokines measured in healthy, control samples, and exemplary profiles of elevated expression levels for each of the subject inflammatory cytokines measured in untreated test subjects.

### Clinical Studies of TPA Compounds for Treating and Preventing ARDS in Humans

Clinical studies regarding anti-viral and anti-ARDS efficacy of TPA compositions and methods in humans will focus directly on SARS-COV-2 positive subjects at elevated risk for, or presenting at onset of study with, one or more symptoms of hyper-inflammation, CSS and/or ARDS, as described. The US Centers of Disease Control and Prevention (CDC) advise diagnosing SARS-COV-2 infection using specimens from both upper respiratory tract (e.g., using nasopharyngeal or oropharyngeal swabbing) and lower respiratory tract (either endotracheal tube or bronchoalveolar lavage). Diagnosis of COVID-19-associated ARDS will be primarily based on the RT-PCR analysis of specimens. If RT-PCR is unavailable, serology tests will be used. Currently, the U.S. Food and Drug Administration (FDA) has approved a SARS-COV-2 commercial test system from Roche (Cobas® SARS-COV-2). This qualitative test requires samples from nasopharyngeal or oropharyngeal swabs, and it take 3.5 h to yield results. Based on RT-PCR methodology, the cobas SARS-COV-2 test is a dual target assay, detecting both the specific SARS-COV-2 RNA, as well as the highly conserved fragment of the E gene invariant in all members of the Sarbecovirus subgenus. The assay has a full-process negative control, positive control and internal control to ensure specificity and accuracy. On 21 Mar. 2020, FDA granted another Emergency Use Authorization to Xpert® Xpress SARS-COV-2 from Cepheid Inc (USA), a qualitative test that can yield results within 45 min. This test can utilize samples from nasopharyngeal swabs, nasal wash, or aspirate specimens and highlights a hands-off, automated sample processing. The results should be viewed as positive if more than one targeted gene is present detected.

While current SARS-COV-2 screening methods rely on abundant viral genome in samples, studies have shown IgM antibody levels are high in both symptomatic and subclinical patients 5 days after onset of illness. Thus, IgM ELISA assays can be combined with PCR to enhance detection sensitivity, to improve both study methods, and practical clinical methods described herein (e.g., to refine determinations for optimizing timing of TPA administration, on a patient-by-patient basis).

Patient management criteria for our human TPA clinical study protocols emphasize the importance of supportive care, to prevention complications and nosocomial transmission. When patients experience respiratory distress, oxygen or respirator support is provided immediately. Unless there are sign of tissue hypoperfusion, fluid resuscitation will be limited, to avoid lung edema and worsening of hypoxemia. Standard precautions, including respiratory and eye protection, are recommended for all study staff. Removal of these precautions is only allowed when a particular study subject shows two consecutive negative RT-PCR tests at least 24 h apart, indicating clinic recovery/clearance of the SARS-COV-2 virus.

An early focus of human clinical trials for TPA in ARDS mediated by SARS-2 will be on consistent and readily assessed diagnostic indices, including radiological features correlated with COVID-19 disease severity. Radiological exam procedures and interpretive protocols for evaluating COVID-19/ARDS severity, including chest X ray (CXR) and chest computed tomography (CT) scan, are well known and widely published, including imaging findings for COVID-19, SARS-COV, and MERS-COV related pneumonia. A large number (up to 75%) of SARS-COV-2 ARDS subjects show bilateral pneumonia, with the remainder unilateral. About 14% of patients show multiple mottling and ground-glass opacities. A predominant pattern of these abnormalities is peripheral (54%), though they may occur in the lower lobes, or be ill-defined. Bilateral multiple consolidation usually occurs in more severe cases.

Chest CT is more efficient in detecting pneumonia at the early stages of COVID-19. The most common patterns of COVID-19 on chest CT scans include multiple GGO lesions (56.4%), and bilateral patchy shadowing (51.8%). Other patterns consist of local patchy shadowing (28.1%), and interstitial abnormalities (4.4%). Severe cases yield more prominent radiologic findings on chest CT scan, such as more bilateral patchy shadowing (82%), more multiple GGO lesions (60%), and more local patchy shadowing (55.1%) than non-severe cases. No CXR or chest CT abnormality was identified in 17.9% of non-severe cases and 2.9% of severe cases. Pure GGO lesions can be found in the early stages. Focal or multifocal GGO lesions may progress into consolidation or GGO lesions with superimposed interlobular/intralobular septal thickening as crazy-paving pattern during disease progression, and the expansion of consolidation represented disease progression. Pure consolidative lesions are relatively less common. Pulmonary cavitary lesion, pleural effusion, and lymphadenopathy are also reported, though rare.

These pulmonary pathogenic data will be collected for TPA treated and control COVID-19/ARDS subjects, along with parallel data for each of the indicia of COVID-19/ARDS severity and TPA efficacy set forth above, as additional human clinical studies proceed. Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes.

### REFERENCES

Arabi Y. M., Alothman A., Balkhy H. H., Al-Dawood A., et al. Treatment of Middle East Respiratory Syndrome with a combination of lopinavir-ritonavir and interferon-β1b (MIRACLE trial): study protocol for a randomized controlled trial. Trials. 2018; 19:1-13.
T. W. Auyeung, J. S. Lee, W. K. Lai, C. H. Choi, H. K. Lee, J. S. Lee, et al., The use of corticosteroid as treatment in SARS was associated with adverse outcomes: a retrospective cohort study, J. Inf. Secur. 51 (2005) 98-102.
Bellingan G., Maksimow M., Howell D. C. et al. The effect of intravenous interferon-beta-1a (FP-1201) on lung CD73 expression and on acute respiratory distress syndrome mortality: an open-label study. Lancet Respir. Med. 2014; 2:98-107.
Bogatcheva N V, Adyshev D, Mambetsariev B, Moldobaeva N, Verin AD. Involvement of microtubules, p38, and Rho kinases pathway in 2-methoxyestradiol-induced lung vascular barrier dysfunction. Arn J Physiol Lung Cell Mo! Physiol. 2007; 292 (2): L487-99.
Caly, L., Druce, J. D., Catton, M. G., et al. The FDA-approved Drug Ivermectin inhibits the replication of SARS-COV-2 in vitro. Antivir. Res. 2020, 104787.
R. C. Chen, X. P. Tang, S. Y. Tan, B. L. Liang, Z. Y. Wan, J. Q. Fang, et al., Treatment of severe acute respiratory syndrome with glucosteroids: the Guangzhou experience, Chest 129 (2006) 1441-1452.
Chan, J. F.-W.; Li, K. S.; To, K. K., et al. Is the discovery of the novel human betacoronavirus 2c EMC/2012 (HCoV-EMC) the beginning of another SARS-like pandemic? J. Infect. 2012, 65, 477-489.
Chan J. F. W., Chan K. H., Kao R. Y. T., To K. K. W., Zheng B. J., Li C. P. Y., Li P. T. W., Dai J., Mok F. K. Y., Chen H., Hayden F. G., Yuen K. Y. Broad-spectrum antivirals for the emerging Middle East respiratory syndrome coronavirus. J. Infect. 2013; 67:606-616.
Chan, J. F.-W.; Lau, S. K.; To, K. K., et al., Middle East Respiratory Syndrome Coronavirus: Another Zoonotic Betacoronavirus Causing SARS-Like Disease. Clin. Microbiol. Rev. 2015A, 28, 465-522.
Chan J. F. W., Yao Y., Yeung M. L., Deng W., Bao L., Jia L., Li F., Xiao C., Gao H., Yu P., Cai J. P., Chu H., Zhou J., Chen H., Qin C., Yuen K. Y. Treatment with lopinavir/ritonavir or interferon-β1b improves outcome of MERSCoV infection in a nonhuman primate model of common marmoset. J. Infect. Dis. 2015B; 212:1904-1913.
Channappanavar R., Fehr A. R., Zheng J. et al. IFN-I response timing relative to virus replication determines MERS coronavirus infection outcomes. J. Clin. Invest. 2019; 129:3625-3639.
Chen F., Chan K. H., Jiang Y., Kao R. Y. T., Lu H. T., Fan K. W., Cheng V. C. C., Tsui W. H. W., Hung I. F. N., Lee T. S. W., Guan Y., Peiris J. S. M., Yuen K. Y. In vitro susceptibility of 10 clinical isolates of SARS coronavirus to selected antiviral compounds. J. Clin. Virol. 2004; 31:69-75.
Chen, J.; Lau, Y. F.; Lamirande, E. W., et al., Cellular Immune Responses to Severe Acute Respiratory Syndrome Coronavirus (SARS-COV) Infection in Senescent BALB/c Mice: CD4+ T Cells Are Important in Control of SARS-COV Infection. J. Virol. 2009, 84, 1289-1301.
J Chun, R Tummala, N Nadiminty, et al. Andrographolide, an Herbal Medicine, Inhibits Interleukin-6 Expression and Suppresses Prostate Cancer Cell Growth. Genes & Cancer 1 (8) 868-876 2010.
B. G. Chousterman, F. K. Swirski, G. F. Weber, Cytokine storm and sepsis disease pathogenesis, Semin. Immunopathol. 39 (2017) 517-528.
Cao, Y.; Li, L.; Feng, Z., et al. Comparative genetic analysis of the novel coronavirus (2019-nCoV/SARS-COV-2) receptor ACE2 in different populations. Cell Discov. 2020, 6, 1-4.
P. Conti, G. Ronconi, A. Caraffa, C. E. Gallenga, R. Ross, I. Frydas, et al., Induction of pro-inflammatory cytokines (IL-1 and IL-6) and lung inflammation by Coronavirus-19 (COVI-19 or SARS-COV-2): anti-inflammatory strategies, J. Biol. Regul. Homeost. Agents 34 (2020), https://doi.org/10.23812/CONTI-E.
De Wit, E.; Van Doremalen, N.; Falzarano, D.; Munster, V. SARS and MERS: Recent insights into emerging coronaviruses. Nat. Rev. Genet. 2016, 14, 523-534.
Duran A, Linares J F, Galvez A S, et al. The signaling adaptor p62 is an important NF-kappaB mediator in tumorigenesis. Cancer Cell. 2008 April; 13(4): 343-54.
Fanelli V, Vlachou A, Ghannadian S, Simonetti U, Siutsky A S, Zhang H. Acute respiratory distress syndrome: new definition, current and future therapeutic options. Journal of thoracic disease. 2013; 5(3) 326-34.
Feng, Z. et al. The novel severe acute respiratory syndrome coronavirus 2 (SARS-COV-2) directly decimates human spleens and lymph nodes. Preprint at medRxiv https://doi.org/10.1101/2020.03.27.20045427 (2020)
Frieman M., Yount B., Heise M., Kopecky-Bromberg S. A., Palese P., Baric R. S. Severe acute respiratory syndrome coronavirus ORF6 antagonizes STAT1 function by sequestering nuclear import factors on the rough endoplasmic reticulum/golgi membrane. J. Virol. 2007; 81:9812-9824.
Guo, R.-F.; Ward, P. A. Role of c5a in inflammatory responses. Annu. Rev. Immunol. 2005, 23, 821-852.
J. C. Ho, G. C. Ooi, T. Y. Mok, J. W. Chan, I. Hung, B. Lam, et al., High-dose pulse versus nonpulse corticosteroid regimens in severe acute respiratory syndrome, Am. J. Respir. Crit. Care Med. 168 (2003) 1449-1456.
C. Huang, Y. Wang, X. Li, L. Ren, J. Zhao, Y. Hu, et al., Clinical features of patients infected with 2019 novel coronavirus in Wuhan, China, Lancet 395 (2020) 497-506.
Hui, D. S.; Zumla, A. Severe Acute Respiratory Syndrome. Infect. Dis. Clin. North Am. 2019, 33, 869-889.
Jolles, S.; Sewell, W. A. C.; Misbah, S. A. Clinical uses of intravenous immunoglobulin. Clin. Exp. Immunol. 2005, 142, 1-11.
Kanne, J. P. Chest CT Findings in 2019 Novel Coronavirus (2019-nCOV) Infections from Wuhan, China: Key Points for the Radiologist. Radiology 2020, 295, 16-17.
Kopecky-Bromberg S. A., Martinez-Sobrido L., Frieman M., Baric R. A., Palese P. Severe acute respiratory syndrome coronavirus open reading frame (ORF) 3b, ORF 6, and nucleocapsid proteins function as interferon antagonists. J. Virol. 2007; 81:548-557.
Lam T T, Shum M H, Zhu H.-C., et al. Identifying SARS-COV-2 related coronaviruses in Malayan pangolins. Nature 2020, 1-6.
Lan, L.; Xu, D.; Ye, G.; Xia, C.; Wang, S.; Li, Y.; Xu, H. Positive RT-PCR Test Results in Patients Recovered From COVID-19. JAMA 2020.
G Lia, X Hea, L Zhanga, et al. Assessing ACE2 expression patterns in lung tissues in the pathogenesis of COVID-19. J. Autoimmunity, Apr. 10, 2020, https://doi.org/10.1016/j.jaut.2020.102463
Lokugamage K. G., Schindewolf C., Menachery V. D. SARS-COV-2 sensitive to type I interferon pretreatment. BioRxiv. 2020 [Google Scholar]
Loutfy M. R., Blatt L. M., Siminovitch K. A., Ward S., Wolff B., Lho H., Pham D. H., Deif H., LaMere E. A., Chang M., Kain K. C., Farcas G. A., Ferguson P., Latchford M., Levy G., Dennis J. W., Lai E. K. Y., Fish E. N. Interferon alfacon-1 plus corticosteroids in severe acute respiratory syndrome: a preliminary study. J. Am. Med. Assoc. 2003; 290:3222-3228.
Lu, R. et al. Lancet 395, 565-574 (2020).
Monteil, H. K., Patricia, P., Astrid, H., et al. Inhibition of SARS-COV-2 infections in engineered human tissues using clinical-grade soluble human ACE2. Cell Press 2020.
Morgenstern B., Michaelis M., Baer P. C., Doerr H. W., Cinatl J. Ribavirin and interferon-β synergistically inhibit SARS-associated coronavirus replication in animal and human cell lines. Biochem. Biophys. Res. Commun. 2005; 326:905-908.
Oeckinghaus A, Ghosh S. The NF-κB Family of Transcription Factors and Its Regulation. Cold Spring Harb Perspect Biol. 2009 October; 1 (4): a000034.
Omrani A. S., Saad M. M., Baig K., Bahloul A., Abdul-Matin M., Alaidaroos A. Y., Almakhlafi G. A., Albarrak M. M., Memish Z. A., Albarrak A. M. Ribavirin and interferon alfa-2a for severe Middle East respiratory syndrome coronavirus infection: a retrospective cohort study. Lancet Infect. Dis. 2014; 14:1090-1095.
Ranieri V. M., Pettilä V., Karvonen M. K., et al. Effect of intravenous interferon β-1a on death and days free from mechanical ventilation among patients with moderate to severe acute respiratory distress syndrome: a randomized clinical trial. JAMA, J. Am. Med. Assoc. 2020; 323:725-733.
Sainz B., Mossel E. C., Peters C. J., Garry R. F. Interferon-beta and interferon-gamma synergistically inhibit the replication of severe acute respiratory syndrome-associated coronavirus (SARS-COV) Virology. 2004; 329:11-17.
E Sallard, F-X Lescure, Y Yazdanpanah, b, F Mentre, N Peiffer-Smadjab, Type 1 interferons as a potential treatment against COVID-19, Antiviral Res. 2020 June; 178:104791, online 2020 Apr. 7. doi: 10.1016/j.antiviral.2020.104791 PMCID: PMC7138382; PMID: 32275914
Scagnolari C., Vicenzi E., Bellomi F., Stillitano M. G., Pinna D., Poli G., Clementi M., Dianzani F., Antonelli G. Increased sensitivity of SARS-coronavirus to a combination of human type I and type II interferons. Antivir. Ther. 2004; 9:1003-1011.
Schneider W. M., Chevillotte M. D., Rice C. M. Interferon-stimulated genes: a complex web of host defenses. Annu. Rev. Immunol. 2014; 32:513-545.
Shen K. L., Yang Y. H. Diagnosis and treatment of 2019 novel coronavirus infection in children: a pressing issue. World J. Pediatr. 2020:6-8.
Sheahan T. P., Sims A. C., Leist S. R., et al. Comparative therapeutic efficacy of remdesivir and combination lopinavir, ritonavir, and interferon beta against MERS-COV. Nat. Commun. 2020; 11
Siddiqi H. K., Mehra M. R. COVID-19 illness in native and immunosuppressed states: a clinical-therapeutic staging proposal. J. Heart Lung Transplant. 2020 [Google Scholar]
Sroussi H Y, Lu Y, Zhang Q L, Villines D, Marucha P T. S100A8 and S100A9 inhibit neutrophil oxidative metabolism in-vitro: involvement of adenosine metabolites. Free radical research. 2010; 44(4): 389-96.
Stockman L. J., Bellamy R., Garner P. SARS: systematic review of treatment effects. PLOS Med. 2006; 3:1525-1531.
Tang, N., Li, D., Wang, X. & Sun, Z. J. Thromb. Haemost. 18, 844-847 (2020).
Thickett, D.; Armstrong, L.; Christie, S. J., et al. Vascular Endothelial Growth Factor May Contribute to Increased Vascular Permeability in Acute Respiratory Distress Syndrome. Am. J. Respir. Crit. Care Med. 2001, 164, 1601-1605.
Totura A. L., Baric R. S. SARS coronavirus pathogenesis: host innate immune responses and viral antagonism of interferon. Curr. Opin. Virol. 2012; 2:264-275.
Y F Tu, C S Chien, A A Yarmishyn, et al. A Review of SARS-COV-2 and the Ongoing Clinical Trials. Int. J. Mol. Sci. 2020, 21, 2657; doi: 10.3390/ijms21072657
Verdoni L, Mazza A, Gervasoni A, Martelli L, Ruggeri M Ciuffreda M et al. An outbreak of severe Kawasaki-like disease at the Italian epicentre of the SARS-COV-2 epidemic: an observational cohort study The Lancet, May 13, 2020, online first DOI: https://doi.org/10.1016/S0140-6736 (20) 31103-X.
Wang, X. et al. Cell Mol. Immunol. https://doi.org/10.1038/s41423-020-0424-9 (2020).
Wang, M.; Cao, R.; Zhang, L., et al. Remdesivir and chloroquine effectively inhibit the recently emerged novel coronavirus (2019-nCOV) in vitro. Cell Res. 2020, 30, 269-271.
Wölfel, R. et al. Nature https://doi.org/10.1038/s41586-020-2196-x (2020).
Xiong, Y. et al. Emerg. Microbes Infect. 9, 761-770 (2020).
Zhang S, Danchuk SD, ImhofK M, et al. Comparison of the therapeutic effects of human and mouse adipose-derived stem cells in a murine model of lipopolysaccharide-induced acute lung injury. Stem Cell Res Ther. 2013; 4(1): 13.
Zhang J, Yang Z, Dong J. P62: An emerging oncotarget for osteolytic metastasis. J Bone Oncol. 2016 Feb. 3; 5(1): 30-7.
Zhang W., Zhao Y., Zhang F., et al. The use of anti-inflammatory drugs in the treatment of people with severe coronavirus disease 2019 (COVID-19): the perspectives of clinical immunologists from China. Clin. Immunol. 2020; 214:108393.
Zhang, Y. et al. N. Engl. J. Med. https://doi.org/10.1056/NEJMc2007575 (2020).
X Zhao 1, J M Nicholls, YG Chen. Severe Acute Respiratory Syndrome-Associated Coronavirus Nucleocapsid Protein Interacts With Smad3 and Modulates Transforming Growth Factor-Beta Signaling. J Biol Chem. 2008 Feb. 8; 283(6): 3272-80. doi: 10.1074/jbc.M708033200. Epub 2007 Nov. 30. PMID: 18055455 DOI: 10.1074/jbc.M708033200
Zheng, M. et al. Cell Mol. Immunol. https://doi.org/10.1038/s41423-020-0402-2 (2020).
Zhong Z, Umemura A, Sanchez-Lopez E, et al., NF-κB Restricts Inflammasome Activation via Elimination of Damaged Mitochondria. Cell. 2016 Feb. 25; 164(5): 896-910.
Zhu, H.; Shi, X.; Ju, D.; Huang, H.; Wei, W.; Dong, X. Anti-Inflammatory Effect of Thalidomide on H1N1 Influenza Virus-Induced Pulmonary Injury in Mice. Inflamm. 2014, 37, 2091-2098.
Zu, Z. Y.; Di Jiang, M.; Xu, P. P. et al., Coronavirus Disease 2019 (COVID-19): A Perspective from China. Radiology 2020, 200490.
Y Zuo, S Yalavarthi, 1 H Shi, et al., Neutrophil Extracellular Traps in Covid-19. Online Apr. 24, 2020 https://insight.jci.org/articles/view/138999

What is claimed:

1. A method for treating or preventing a sudden acute respiratory syndrome (SARS) coronavirus infection in a mammalian subject comprising: administering an anti-viral effective amount of a TPA compound to a subject at elevated risk for SARS coronavirus infection, or presenting with active SARS coronavirus infection, to elicit a significant, clinically therapeutic or prophylactic, anti-viral response in the subject, sufficient to prevent or reduce SARS viral infection and/or reduce or eliminate SARS viral load detectable in the subject.

2. The method of claim 1, wherein the SARS coronavirus is a human SARS (hSARS) coronavirus selected from SARS-COV-2 (COVID-19), SARS-COV, and middle east respiratory syndrome (MERS) coronaviruses.

3. The method of claim 2, wherein the hSARS coronavirus is SARS-COV-2 (COVID-19).

4. The method of claim 1, wherein the anti-viral TPA compound is effective to elicit an anti-viral response in the subject sufficient to prevent or reduce SARS-COV-2 viral infection and/or reduce or eliminate SARS viral load compound, and to concurrently prevent or reduce one or more clinical symptoms of COVID-19 disease.

5. The method of claim 1, wherein the TPA compound is administered in an amount and dosage form effective to reduce or eliminate one or more indicia of SARS coronavirus infection severity selected from: 1) viral load or titer in an upper or lower respiratory cell, tissue or sample of the subject; 2) viral load or titer in a non-respiratory, ACE-2 positive cell, tissue or sample of the subject, or in a blood plasma of the subject; 3) viral attachment and/or entry into lung or other tissues/cells; 4) viral replication in a lung or other ACE-2 positive cell, tissue or organ of the subject; and/or 5) viral shedding from an upper respiratory tract tissue or sample of an infected subject, each of said indices determinable by observing or measuring an incidence or value of the subject index in one or more TPA-treated subjects in comparison to incidence or value of the same index in one or more comparable, placebo-treated control subject(s).

6. The method of claim 1, wherein the SARS coronavirus is a human SARS (hSARS) coronavirus selected from SARS-COV-2 (COVID-19), SARS-COV, and middle east respiratory syndrome (MERS) coronaviruses, and wherein the TPA compound is effective to reduce hSARS viral load or titer in a an upper and/or lower respiratory tract of the subject.

7. The method of claim 1, wherein the SARS virus is a human SARS (hSARS) coronavirus selected from SARS-COV-2 (COVID-19), SARS-COV, and middle east respiratory syndrome (MERS) coronaviruses, and wherein the TPA compound is effective to reduce hSARS viral load or titer in a non-respiratory, ACE-2 positive cell, tissue or organ of the subject.

8. The method of claim 1, wherein the SARS virus is SARS-COV-2 (COVID-19), and wherein the anti-viral TPA compound is effective to elicit an anti-viral response in the subject that prevents or reduces SARS-COV-2 viral infection and/or reduces or eliminates SARS viral load, as determined by SARS-COV-2 DNA or other quantitative measure of SARS-COV-2 levels in nasopharyngeal swab samples taken pre- and post-treatment from infected subjects, or by comparison of suitable test and control samples.

9. The method of claim 8, wherein viral load as determined by SARS-COV-2 DNA or other quantitative measure of SARS-COV-2 viral load in nasopharyngeal swab samples, compared between pre- and post-treatment samples from an individual or group of treated patient(s), or between treated and placebo-treated control subjects, is decreased by an average of 25-50% or more among treated subjects.

10. The method of claim 8, wherein viral load as determined by SARS-COV-2 DNA or other quantitative measure of SARS-COV-2 viral load in nasopharyngeal swab samples, compared between pre- and post-treatment samples from an individual or group of treated patient(s), or between treated and placebo-treated control subjects, is decreased by an average of 75-95% or more among treated subjects.

11. The method of claim 8, wherein viral load as determined by SARS-COV-2 DNA or other quantitative measure of SARS-COV-2 load in nasopharyngeal swab samples from subjects screened as positive for SARS-COV-2 infection before TPA treatment, is decreased within two weeks after TPA treatment by 100% (corresponding to total clearance of detectable SARS-COV-2 virus in the upper respiratory tract, indicative of a non-contagious status) in at least 50% of TPA-treated subjects.

12. The method of claim 1, wherein the anti-viral TPA compound is selected from compounds of Formula I, below, and anti-viral active analogs, derivatives, complexes, conjugates, salts, enantiomers and mixtures thereof: 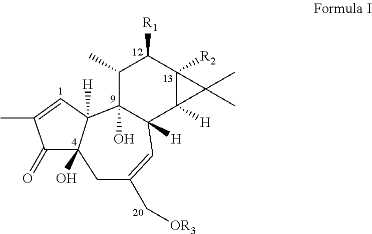
wherein R₁ and R₂ are selected from the group consisting of hydrogen; 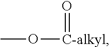
wherein the alkyl group contains 1 to 15 carbon atoms, 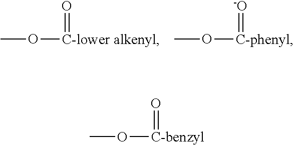
and substituted derivatives thereof and R₃ may be hydrogen, 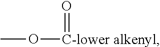
or substituted derivatives thereof,
wherein the “lower alkyl” or “lower alkenyl” can contain 1-7 carbons and may be straight or branched, and are optionally unsubstituted or substituted by chlorine, fluorine, or another halogen, or nitro, amino or other active functionality.

13. The method of claim 1, wherein the anti-viral TPA compound is selected from Formula I below and anti-viral active analogs, derivatives, complexes, conjugates, salts, enantiomers and mixtures thereof.

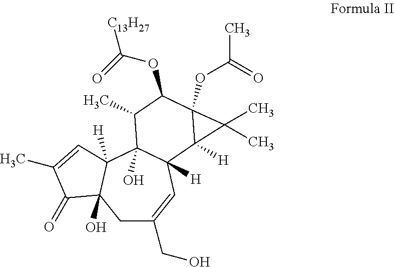

14. The method of claim 1, wherein the anti-viral TPA compound is a phorbol ester selected from: phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; phorbol 13-acetate; and anti-viral active analogs, derivatives, complexes, conjugates, salts, enantiomers and mixtures thereof.

15. The method of claim 1, wherein the anti-viral TPA compound is 12-O-tetradecanoylphorbol-13-acetate.

16. The method of claim 1, wherein the SARS virus is SARS-COV-2 (COVID-19), and wherein the anti-viral TPA compound is first administered within 2 weeks of a subject being initially diagnosed with SARS-COV-2 infection.

17. The method of claim 1, wherein the SARS virus is SARS-COV-2 (COVID-19), and wherein the anti-viral TPA compound is first administered 7-10 days after the subject is initially diagnosed with SARS-COV-2 infection.

18. The method of claim 1, wherein the SARS virus is SARS-COV-2 (COVID-19), and wherein the anti-viral TPA compound is administered before a subject at elevated-risk or known-infected subject manifests one or more index (ices) of severe COVID-19 disease selected from: 1) fever lasting over 2 days; 2) lower respiratory symptoms of pulmonary congestion, tightness, shortness of breath and/or hypoxemia; 3) a condition or symptom associated with acute respiratory distress syndrome (ARDS), including cytokine storm syndrome (CSS); Extrapulmonary Systemic Hyperinflammation Syndrome (ESHS), and/or 4) another condition or symptom associated with a severe hyper-immune or hyper-inflammatory response in the subject, including Pediatric Inflammatory Multisystem Syndrome (PIMS); vascular congestive and thrombotic conditions, Disseminated Intravascular Coagulation (DIC), thrombosis, stroke, and/or thrombocytopenia.

19. The method of claim 1, wherein the anti-viral TPA compound is formulated and administered simultaneously with, or coordinately administered simultaneously or sequentially in a coordinate, multi-drug prophylactic or treatment protocol with, a secondary therapeutic or prophylactic drug or agent selected from: 1) a conventional anti-viral drug or agent; 2) an anti-ARDS drug or agent; 3) an anti-CSS drug or agent, 4) an anti-PIMS drug or agent; 5) an anti-ESHS drug or agent; 6) an anti-DAD drug or agent; and/or 7) an anti-inflammatory, pro-immune, anti-cytopathic and/or pro-apoptotic drug or agent; and combinations thereof.

20. The method of claim 8, wherein the anti-SARS-COV2 TPA compound is formulated and administered simultaneously with, or coordinately administered simultaneously or sequentially in a coordinate, multi-drug prophylactic or treatment protocol with, a secondary anti-viral drug or agent.

21. The method of claim 20, wherein the secondary anti-viral drug or agent is selected from: Abacavir, Acyclovir, Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine, and combinations thereof.

22. The method of claim 20, wherein the secondary anti-viral drug or agent is an anti-ACE2 drug or agent.

23. The method of claim 20, wherein the secondary anti-viral drug or agent is an anti-inflammatory drug or agent.

24. The method of claim 23, wherein the anti-inflammatory drug or agent is selected from non-steroidal anti-inflammatory drugs (NSAIDs).

25. The method of claim 24, wherein the NSAIDs include: aspirin, celecoxib (Celebrex), diclofenac (Cambia, Cataflam, Voltaren-XR, Zipsor, Zorvolex), diflunisal, etodolac, ibuprofen (Motrin, Advil), indomethacin (Indocin), celecoxib (Celebrex), piroxicam (Feldene), indomethacin (Indocin), meloxicam (Mobic Vivlodex), ketoprofen (Orudis, Ketoprofen ER, Oruvail, Actron), sulindac (Clinoril), diflunisal (Dolobid), nabumetone (Relafen), oxaprozin (Daypro), tolmetin (Tolmetin Sodium, Tolectin), salsalate (Disalcid), fenoprofen (Nalfon), flurbiprofen (Ansaid), ketorolac (Toradol), meclofenamate, mefenamic acid (Ponstel), and combinations thereof.

26. The method of claim 20, wherein the secondary anti-viral drug or agent is a cytokine inhibitor drug or agent.

27. The method of claim 26, wherein the cytokine inhibitor drug or agent is effective to inhibit or lower induction, synthesis, activation and/or circulating level(s) of one or more pro-inflammatory cytokine targets selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.xx

28. The method of claim 27, wherein coordinate multi-drug therapy with the TPA compound and cytokine inhibitor drug is combinatorially effective to yield improved, additive, synergistic and/or potentiating therapeutic benefits (compared to benefits yielded by either drug/agent alone in a same dosage) for reducing induction, synthesis, activation and/or circulating level(s) of one or more pro-inflammatory cytokine targets that are hyper-elevated in association with SARS-CoV-2 infection, COVID-19 disease, ARDS, SARS, CSS, PIMS, ESHS, DAD or another hyper-inflammatory condition mediated or exacerbated by SARS-COV-2 infection.

29. The method of claim 20, wherein the secondary anti-viral drug or agent is an anti-IL-6 drug or biologic agent.

30. The method of claim 29, wherein the anti-IL-6 drug or biologic agent is an anti-IL-6 monoclonal antibody or Fab fragment, a soluble IL-6 receptor or receptor analog, or a cognate anti-IL-6 specific binding or deactivating domain thereof.

31. The method of claim 30, wherein the anti-IL-6 drug or biologic is selected from siltuximab, sarilumab (Kevzara), and tocilizumab (TCZ).

32. The method of claim 29, wherein the anti-IL-6 drug or biologic is an anti-IL-6 drug that blocks or inhibits IL-6 directly, or indirectly inhibits, lowers, or alters a pro-inflammatory activity of IL-6.

33. The method of claim 32, wherein the anti-IL-6 drug is andrographolide.

34. The method of claim 20, wherein the secondary anti-viral drug or agent is a kinase modulating drug or agent.

35. The method of claim 34, wherein the kinase inhibitor is a kinase modulating drug or agent directly or indirectly inhibits, lowers, activates or alters an immune or inflammatory activity of one or more kinases involved in mediating or suppressing inflammatory responses, or regulating differentiation, proliferation, activation, inflammatory cytokine synthesis, and/or apoptotic activity of immune and/or inflammatory effector cells, including lymphocytes, monocyte/macrophage cells and/or neutrophils.

36. The method of claim 35, wherein the kinase modulator drug modulates a mitogen activated protein kinase (MAPK), janus kinase (JAK) and/or protein kinase C (PKC).

37. The method of claim 36, wherein coordinate treatment of COVID-19 disease subjects with the TPA compound and kinase modulator drug clinically reduces one or more disease condition(s) or symptom(s) associated with severe SARS-COV-2 infection, including one or more condition(s) or symptom(s) associated with ARDS, SARS, CSS, PIMS, ESHS, and DAD.

38. The method of claim 20, wherein the secondary anti-viral drug or agent is an anti-SARS-CoV-2 vaccine agent.

39. The method of claim 20, wherein the secondary anti-viral drug or agent is a composition comprising conditioned natural killer (NK) Cells.

40. The method of claim 20, wherein the secondary anti-viral drug or agent is a composition comprising conditioned mesenchymal stem cells (MSCs).

41. The method of claim 20, wherein the secondary anti-viral drug or agent is a recombinant Interferon.

42. The method of claim 20, wherein the secondary anti-viral drug or agent is an intravenous formulated immunoglobulin.

43. The method of claim 20, wherein the secondary anti-viral drug or agent comprises a SARS-COV-2-specific neutralizing antibody, Fab fragment or antibody binding domain.

44. The method of claim 20, wherein the secondary anti-viral drug or agent comprises a C5a-specific antibody, Fab fragment or antibody binding domain.

45. The method of claim 20, wherein the secondary anti-viral drug or agent is selected from Thalidomide, Fingolimod, anti-angiogenic drugs, hydroxychloroquine and glucocorticoids. The method of claim 1, which is anti-virally effective to mediate one or more significant clinical benefits relating to prevention and/or treatment of SARS-COV-2 viral infection, selected from: 1) preventing or reducing viral infection or titer in the upper respiratory tract; 2) preventing or reducing viral infection or titer the lower respiratory tract; 3) preventing or reducing viral infection or titer in non-respiratory, ACE-2 positive cell and tissues; 4) preventing or reducing viral attachment and entry into lung and other ACE-2 positive cells and tissues; 5) preventing or reducing viral replication in lung and other ACE-2 positive cell and tissues; and/or 7) preventing or reducing viral shedding from an upper respiratory tract of infected subjects.

46. A method for treating an acute respiratory distress syndrome (ARDS) in a mammalian subject, comprising: administering an anti-ARDS effective amount of a TPA composition to said subject, sufficient to prevent, reduce or eliminate of one or more ARDS disease condition(s) and/or symptom(s) selected from 1) dyspnea; 2) hyper-elevated level(s) of one or more pro-inflammatory cytokine(s) in the lung; 3) hyper-elevated level(s) of monocyte/macrophage cells and/or neutrophils in a lung parenchyma and/or a pulmonary alveolar compartment; 4) degradation or disruption of a pulmonary endothelial and/or epithelial barrier(s); 5) elevated indicia of oxidative stress in lung tissue, determinable by elevated levels of reactive oxygen species (ROS) in the lung; and/or 6) one or more pathogenic symptom(s) of lung injury selected from hyper-inflammation, fibrosis, diffuse alveolar damage (DAD), macrophage and/or neutrophil infiltration into the lung parenchyma, macrophage and/or neutrophil infiltration into pulmonary capillaries, deposition of extensive neutrophil extracellular traps (NETs) in a lung interstitium or parenchyma, pulmonary and/or coronary vessel thromboses, and vasculitis in a treated subject in comparison to the same ARDS disease indicator(s)/value(s) measured and determined in comparable, placebo-treated control subjects.

47. The method of claim 46, wherein the ARDS is caused by a respiratory viral or bacterial infection, heat or chemical burn injury to the lungs, pulmonary trauma, or another disease or injury that triggers an immune dysfunction or hyper-inflammatory response that mediates extensive pulmonary injury and dysfunction.

48. The method of claim 46, wherein the ARDS is caused by a sudden acute respiratory syndrome (SARS) coronavirus.

49. The method of claim 48, wherein the SARS coronavirus is a human SARS (hSARS) coronavirus selected from SARS-COV-2 (COVID-19), SARS-COV, and middle east respiratory syndrome (MERS) coronaviruses.

50. The method of claim 46, wherein the anti-ARDS TPA compound is effective to prevent or reduce an incidence or severity of dyspnea (labored, short or inadequate breathing), hypoxemia and/or required respirator support in ARDS-affected subjects.

51. The method of claim 46, wherein the anti-ARDS TPA compound is effective to prevent or reduce a hyper-elevated level and/or activity of one or more pro-inflammatory cytokine(s) in the lung, plasma or other cell, tissue or compartment linked to ARDS-associated hyper-inflammation.

52. The method of claim 46, wherein the one or more pro-inflammatory cytokine(s) is/are selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.

53. The method of claim 46, wherein the anti-ARDS TPA compound is effective to prevent or reduce hyper-elevated level(s) of monocyte/macrophage cells and/or neutrophils in a lung parenchyma and/or a pulmonary alveolar compartment in ARDS-affected subjects.

54. The method of claim 53, wherein the anti-ARDS TPA compound is effective to prevent or reduce hyper-elevated levels of neutrophils and deposition of associated neutrophil extracellular traps (NETs) in a lung parenchyma, pulmonary alveolar compartment and/or pulmonary blood vessels in ARDS-affected subjects.

55. The method of claim 46, wherein the anti-ARDS TPA compound is effective to prevent or reduce degradation or disruption of a pulmonary endothelial and/or epithelial barrier(s) in ARDS-affected subjects.

56. The method of claim 46, wherein the anti-ARDS TPA compound is effective to prevent or reduce oxidative stress in lung tissue of ARDS-affected subjects, including by reducing reactive oxygen species (ROS) in the lung.

57. The method of claim 46, wherein the anti-ARDS TPA compound is effective to prevent or reduce the extent of a lung injury selected from hyper-inflammation, fibrosis, diffuse alveolar damage (DAD), macrophage and/or neutrophil infiltration into the lung parenchyma, macrophage and/or neutrophil infiltration into pulmonary capillaries, deposition of extensive neutrophil extracellular traps (NETs) in a lung interstitium or parenchyma, pulmonary and/or coronary vessel thromboses, and vasculitis in ARDS-affected subjects.

58. The method of claim 46, wherein the anti-ARDS TPA compound is selected from Formula I below and anti-ARDS active analogs, derivatives, complexes, conjugates, salts, enantiomers and mixtures thereof.

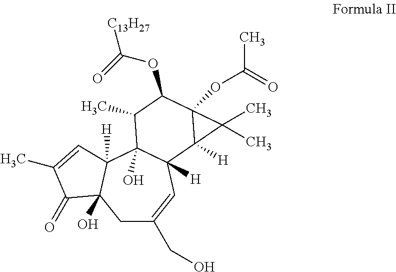

59. The method of claim 46, wherein the anti-ARDS TPA compound is a phorbol ester selected from: phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; phorbol 13-acetate; and anti-ARDS active analogs, derivatives, complexes, conjugates, salts, enantiomers and mixtures thereof.

60. The method of claim 46, wherein the anti-ARDS TPA compound is 12-O-tetradecanoylphorbol-13-acetate.

61. The method of claim 46, wherein the ARDS is caused by a sudden acute respiratory syndrome (SARS)-2 (SARS-COV-2 or COVID-19) coronavirus, and wherein the anti-ARDS TPA compound is first administered within 2 weeks of a subject being initially diagnosed with SARS-COV-2 infection.

62. The method of claim 46, wherein the ARDS is caused by a sudden acute respiratory syndrome (SARS)-2 (SARS-COV-2 or COVID-19) coronavirus, and wherein the anti-ARDS TPA compound is first administered 7-10 days after the subject is initially diagnosed with SARS-COV-2 infection.

63. The method of claim 46, wherein the ARDS is caused by a sudden acute respiratory syndrome (SARS)-2 (SARS-COV-2 or COVID-19) coronavirus, and wherein the anti-ARDS TPA compound is administered before an elevated-risk or known-infected subject manifests one or more index(ices) of severe COVID-19 disease selected from: 1) fever lasting over 2 days; 2) lower respiratory symptoms of pulmonary congestion, tightness, shortness of breath and/or hypoxemia; 3) a condition or symptom associated with ARDS selected from: cytokine storm syndrome (CSS); Extrapulmonary Systemic Hyperinflammation Syndrome (ESHS), and/or 4) any other condition or symptom mediated by a severe hyper-immune or hyper-inflammatory response in the subject, including Pediatric Inflammatory Multisystem Syndrome (PIMS), vascular congestive and thrombotic conditions, Disseminated Intravascular Coagulation (DIC), thrombosis, stroke, and/or thrombocytopenia.

64. The method of claim 46, wherein the anti-ARDS TPA compound is effective to treat one or more disease condition(s) or symptom(s) associated with (ARDS) selected from 1) lower respiratory symptoms of pulmonary congestion, tightness, shortness of breath and/or hypoxemia; 2) cytokine storm syndrome (CSS); 3) Extrapulmonary Systemic Hyperinflammation Syndrome (ESHS), and/or 4) another condition or symptom mediated by a severe hyper-immune or hyper-inflammatory response in the subject, including Pediatric Inflammatory Multisystem Syndrome (PIMS), vascular congestive and thrombotic conditions, Disseminated Intravascular Coagulation (DIC), thrombosis, stroke, and/or thrombocytopenia.

65. The method of claim 46, wherein the anti-ARDS TPA compound is formulated and administered simultaneously with, or coordinately administered simultaneously or sequentially in a coordinate, multi-drug prophylactic or treatment protocol with, a secondary therapeutic or prophylactic drug or agent selected from: 1) a conventional anti-viral drug or agent; 2) a secondary anti-ARDS drug or agent; 3) an anti-CSS drug or agent, 4) an anti-PIMS drug or agent; 5) an anti-ESHS drug or agent; 6) an anti-DAD drug or agent; and/or 7) an anti-inflammatory, pro-immune, anti-cytopathic and/or pro-apoptotic drug or agent; and combinations thereof.

66. The method of claim 65, wherein the ARDS is caused by a sudden acute respiratory syndrome (SARS)-2 (SARS-COV-2 or COVID-19) coronavirus, and wherein the anti-ARDS TPA compound is formulated and administered simultaneously with, or coordinately administered simultaneously or sequentially in a coordinate, multi-drug prophylactic or treatment protocol with, a secondary anti-viral drug or agent.

67. The method of claim 66, wherein the secondary anti-viral drug or agent is selected from: Abacavir, Acyclovir, Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine, and combinations thereof.

68. The method of claim 65, wherein the secondary drug or agent is an anti-ACE2 drug or agent.

69. The method of claim 65, wherein the secondary drug or agent is an anti-inflammatory drug or agent.

70. The method of claim 69, wherein the anti-inflammatory drug or agent is selected from non-steroidal anti-inflammatory drugs (NSAIDs).

71. The method of claim 70, wherein the NSAIDs include: aspirin, celecoxib (Celebrex), diclofenac (Cambia, Cataflam, Voltaren-XR, Zipsor, Zorvolex), diflunisal, etodolac, ibuprofen (Motrin, Advil), indomethacin (Indocin), celecoxib (Celebrex), piroxicam (Feldene), indomethacin (Indocin), meloxicam (Mobic Vivlodex), ketoprofen (Orudis, Ketoprofen ER, Oruvail, Actron), sulindac (Clinoril), diflunisal (Dolobid), nabumetone (Relafen), oxaprozin (Daypro), tolmetin (Tolmetin Sodium, Tolectin), salsalate (Disalcid), fenoprofen (Nalfon), flurbiprofen (Ansaid), ketorolac (Toradol), meclofenamate, mefenamic acid (Ponstel), and combinations thereof.

72. The method of claim 65, wherein the secondary drug or agent is a cytokine inhibitor drug or agent.

73. The method of claim 72, wherein the cytokine inhibitor drug or agent is effective to inhibit or lower induction, synthesis, activation and/or circulating level(s) of one or more pro-inflammatory cytokine targets selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.

74. The method of claim 72, wherein the ARDS is caused by a sudden acute respiratory syndrome (SARS)-2 (SARS-COV-2 or COVID-19) coronavirus, and wherein coordinate multi-drug therapy with the TPA compound and cytokine inhibitor drug is combinatorially effective to yield improved, additive, synergistic and/or potentiating therapeutic benefits (compared to benefits yielded by either drug/agent alone in a same dosage) for reducing induction, synthesis, activation and/or circulating level(s) of one or more pro-inflammatory cytokine targets that are hyper-elevated in association with SARS-COV-2 infection, COVID-19 disease, ARDS, SARS, CSS, PIMS, ESHS, DAD or another hyper-inflammatory condition mediated or exacerbated by SARS-COV-2 infection.

75. The method of claim 65, wherein the secondary drug or agent is an anti-IL-6 drug or biologic agent.

76. The method of claim 75, wherein the anti-IL-6 drug or biologic agent is an anti-IL-6 monoclonal antibody or Fab fragment, a soluble IL-6 receptor or receptor analog, or a cognate anti-IL-6 specific binding or deactivating domain thereof.

77. The method of claim 75, wherein the anti-IL-6 drug or biologic is selected from siltuximab, sarilumab (Kevzara), and tocilizumab (TCZ).

78. The method of claim 75, wherein the anti-IL-6 drug or biologic is an anti-IL-6 drug that blocks or inhibits IL-6 directly, or indirectly inhibits, lowers, or alters a pro-inflammatory activity of IL-6.

79. The method of claim 78, wherein the anti-IL-6 drug is andrographolide.

80. The method of claim 65, wherein the secondary drug or agent is a kinase modulating drug or agent.

81. The method of claim 80, wherein the kinase modulating drug or agent directly or indirectly inhibits, lowers, activates or alters an immune or inflammatory activity of one or more kinases involved in mediating or suppressing inflammatory responses, or regulating differentiation, proliferation, activation, inflammatory cytokine synthesis, and/or apoptotic activity of immune and/or inflammatory effector cells, including lymphocytes, monocyte/macrophage cells and/or neutrophils.

82. The method of claim 81, wherein the kinase modulator drug or agent modulates a mitogen activated protein kinase (MAPK), janus kinase (JAK) and/or protein kinase C (PKC).

83. The method of claim 65, wherein the ARDS is caused by a sudden acute respiratory syndrome (SARS)-2 (SARS-COV-2 or COVID-19) coronavirus, and wherein the coordinate treatment of COVID-19 disease subjects with the TPA compound and kinase modulator drug clinically reduces one or more disease condition(s) or symptom(s) associated with severe SARS-CoV-2 infection, including one or more condition(s) or symptom(s) associated with ARDS, SARS, CSS, PIMS, ESHS, and DAD.

84. The method of claim 65, wherein the ARDS is caused by a sudden acute respiratory syndrome (SARS)-2 (SARS-COV-2 or COVID-19) coronavirus, and wherein the secondary drug or agent is an anti-SARS-COV-2 vaccine agent.

85. The method of claim 65, wherein the secondary drug or agent is a composition comprising conditioned natural killer (NK) Cells.

86. The method of claim 65, wherein the secondary drug or agent is a composition comprising conditioned mesenchymal stem cells (MSCs).

87. The method of claim 65, wherein the secondary drug or agent is a recombinant Interferon.

88. The method of claim 65, wherein the ARDS is caused by a sudden acute respiratory syndrome (SARS)-2 (SARS-COV-2 or COVID-19) coronavirus, and wherein the secondary drug or agent is an intravenous formulated immunoglobulin.

89. The method of claim 65, wherein the ARDS is caused by a sudden acute respiratory syndrome (SARS)-2 (SARS-COV-2 or COVID-19) coronavirus, and wherein the secondary drug or agent comprises a SARS-COV-2-specific neutralizing antibody, Fab fragment or antibody binding domain.

90. The method of claim 65, wherein the secondary drug or agent comprises a C5a-specific antibody, Fab fragment or antibody binding domain.

91. The method of claim 65, wherein the secondary drug or agent is selected from Thalidomide, Fingolimod, anti-angiogenic drugs, hydroxychloroquine and glucocorticoids.

92. The method of claim 65, wherein the ARDS is caused by a sudden acute respiratory syndrome (SARS)-2 (SARS-COV-2 or COVID-19) coronavirus, and wherein anti-ARDS TPA compound and secondary drug or agent are combinatorially effective to mediate one or more significant clinical benefits relating to prevention and/or treatment of SARS-COV-2 viral infection and COVID-19 disease selected from: 1) preventing or reducing viral infection or titer in the upper respiratory tract; 2) preventing or reducing viral infection or titer the lower respiratory tract; 3) preventing or reducing viral infection or titer in non-respiratory, ACE-2 positive cell and tissues; 4) preventing or reducing viral attachment and entry into lung and other ACE-2 positive cells and tissues; 5) preventing or reducing viral replication in lung and other ACE-2 positive cell and tissues; and/or 7) preventing or reducing viral shedding from an upper respiratory tract of infected subjects.

93. A method for treating an immune dysfunction or hyper-inflammatory condition in a mammalian subject suffering from a Cytokine Storm Syndrome (CSS), Pediatric Inflammatory Multisystem Syndrome (PIMS), Extrapulmonary Systemic Hyperinflammation Syndrome (ESHS) generally, or a vascular congestive or thrombotic condition caused by hyperinflammation, including Disseminated Intravascular Coagulation (DIC), thrombosis, stroke, thrombocytopenia, and/or gangrene, comprising: administering an anti-inflammatory effective amount of a TPA compound to the subject.

94. The method of claim 93, wherein the subject is at elevated risk for or is infected with sudden acute respiratory syndrome (SARS)-2 (SARS-COV-2 or COVID-19) coronavirus, and wherein the anti-inflammatory TPA compound is effective to mediate an anti-CSS response in the subject.

95. The method of claim 94, wherein the anti-CSS response includes prevention or reduction of one or more CSS-associated condition(s) or symptom(s) selected from: 1) hyper-elevated level(s) of one or more pro-inflammatory cytokine(s) in the lung or another tissue or organ site of hyper-inflammation; 2) hyper-elevated level(s) of monocyte/macrophage cells and/or neutrophils in a lung parenchyma, pulmonary alveolar compartment or other tissue or organ site of hyper-inflammation; 3) degradation or disruption of endothelial and/or epithelial barrier(s) in the lung or other tissue or organ site of hyper-inflammation; 4) elevated indicia of oxidative stress in the lung or other tissue or organ site of hyper-inflammation, determinable by elevated levels of reactive oxygen species (ROS); and/or 5) one or more pathogenic symptom(s) of tissue or organ injury selected from hyper-inflammation, fibrosis, diffuse alveolar damage (DAD), macrophage and/or neutrophil infiltration into the lung parenchyma or other tissue or organ site of hyper-inflammation, macrophage and/or neutrophil infiltration into capillaries of the lung or another tissue or organ site of hyper-inflammation, deposition of extensive neutrophil extracellular traps (NETs) in a lung interstitium or parenchyma or other tissue or organ site of hyper-inflammation, pulmonary and/or coronary vessel thromboses, and/or vasculitis in treated subjects.

96. The method of claim 93, wherein the subject presents with CSS and the TPA compound is an anti-CSS effective TPA compound that elicits at least a 25% reduction in one or more condition(s), symptom(s) or diagnostic index (ices) associated with CSS selected from 1) hyper-elevated pro-inflammatory cytokine activation, expression and/or levels in CSS-affected cells or tissues; 2) increased infiltration and/or elevated numbers of macrophages and/or neutrophils in the lung parenchyma, pulmonary alveolar airspaces, or another CSS-affected tissue or organ; 3) lymphocytopenia marked by numerical decline of lymphocytes; 4) elevated oxidative stress markers; 5) inflammatory injury to endothelial and/or epithelial barriers in the lungs or another CSS-affected tissue or organ; 6) pathogenic fibrosis or other pathologic inflammatory injury to the lungs or another CSS-affected tissue or organ/organ; 7) inflammatory injury, loss or atrophy of lymph nodes; 8) splenic inflammatory injury or atrophy; 9) Sepsis; 10) Toxic Shock Syndrome (TSS); 11) oxidative stress symptoms; and/or 12) one or more pathogenic symptom(s) of tissue or organ injury selected from hyper-inflammation, fibrosis, diffuse alveolar damage (DAD), macrophage and/or neutrophil infiltration into the lung parenchyma or other tissue or organ site of hyper-inflammation, macrophage and/or neutrophil infiltration into capillaries of the lung or another tissue or organ site of hyper-inflammation, deposition of extensive neutrophil extracellular traps (NETs) in a lung interstitium or parenchyma or other tissue or organ site of hyper-inflammation, pulmonary and/or coronary vessel thromboses, and/or vasculitis in treated subjects (wherein each indicator/value is measured and determined in treated subjects, in comparison to the same indicator/value measured and determined in similar, placebo-treated control subjects).

97. The method of claim 96, wherein the anti-CSS TPA compound is effective to prevent or reduce dysregulation and hyper-elevation of pro-inflammatory cytokines associated with CSS, wherein treated subjects show at least a 25% reduction in hyper-elevated level(s) of one or more pro-inflammatory cytokine(s).

98. The method of claim 94, wherein the anti-inflammatory TPA compound is effective to prevent or reduce a hyper-elevated level and/or activity of one or more pro-inflammatory cytokine(s) in the lung, plasma or other cell, tissue or compartment linked to ARDS-associated hyper-inflammation.

99. The method of claim 98, wherein the one or more pro-inflammatory cytokine(s) is/are selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.

100. The method of claim 94, wherein the anti-inflammatory TPA compound is effective to prevent or reduce hyper-elevated level(s) of monocyte/macrophage cells and/or neutrophils in a lung parenchyma, pulmonary alveolar compartment or other tissue or organ site of hyper-inflammation in CSS-affected subjects.

101. The method of claim 94, wherein the anti-inflammatory TPA compound is effective to prevent or reduce hyper-elevated levels of neutrophils and deposition of associated neutrophil extracellular traps (NETs) in a lung parenchyma, pulmonary alveolar compartment, pulmonary blood vessels or other tissue or organ site of hyper-inflammation in CSS-affected subjects.

102. The method of claim 94, wherein the anti-inflammatory TPA compound is effective to prevent or reduce degradation or disruption of endothelial and/or epithelial barrier(s) in the lungs or other tissue or organ site of hyper-inflammation in CSS-affected subjects.

103. The method of claim 94, wherein the anti-inflammatory TPA compound is effective to prevent or reduce oxidative stress in a lung or other tissue or organ site of hyper-inflammation in CSS-affected subjects.

104. The method of claim 94, wherein the anti-inflammatory TPA compound is effective to prevent or reduce the extent of a hyper-inflammatory tissue or organ injury selected from hyper-inflammation, fibrosis, diffuse alveolar damage (DAD), macrophage and/or neutrophil infiltration into the lung parenchyma or other tissue or organ site of hyper-inflammation, macrophage and/or neutrophil infiltration into capillaries of the lung or other tissue or organ site of hyper-inflammation, deposition of extensive neutrophil extracellular traps (NETs) in a lung interstitium or parenchyma or other tissue or organ site of hyper-inflammation, pulmonary and/or coronary vessel thromboses, and vasculitis in CSS-affected subjects.

105. The method of claim 94, wherein the anti-inflammatory TPA compound is selected from Formula I below and anti-inflammatory active analogs, derivatives, complexes, conjugates, salts, enantiomers and mixtures thereof.

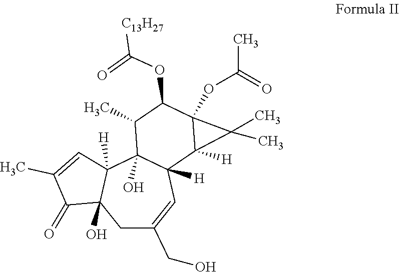

106. The method of claim 94, wherein the anti-inflammatory TPA compound is a phorbol ester selected from: phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; phorbol 13-acetate; and anti-inflammatory active analogs, derivatives, complexes, conjugates, salts, enantiomers and mixtures thereof.

107. The method of claim 94, wherein the anti-inflammatory TPA compound is 12-O-tetradecanoylphorbol-13-acetate.

108. The method of claim 94, wherein the wherein the anti-inflammatory TPA compound is first administered within 2 weeks of a subject being initially diagnosed with SARS-COV-2 infection.

109. The method of claim 94, wherein the anti-inflammatory TPA compound is first administered 7-10 days after the subject is initially diagnosed with SARS-COV-2 infection.

110. The method of claim 94, wherein the anti-inflammatory TPA compound is administered before an elevated-risk or known-infected subject manifests one or more index (ices) of severe COVID-19 disease selected from: 1) fever lasting over 2 days; 2) lower respiratory symptoms of pulmonary congestion, tightness, shortness of breath and/or hypoxemia; 3) a condition or symptom associated with ARDS selected from: cytokine storm syndrome (CSS); Extrapulmonary Systemic Hyperinflammation Syndrome (ESHS), and/or 4) any other condition or symptom mediated by a severe hyper-immune or hyper-inflammatory response in the subject, including Pediatric Inflammatory Multisystem Syndrome (PIMS), vascular congestive and thrombotic conditions, Disseminated Intravascular Coagulation (DIC), thrombosis, stroke, and/or thrombocytopenia.

111. The method of claim 94, wherein the anti-inflammatory TPA compound is effective to treat one or more disease condition(s) or symptom(s) associated with acute respiratory distress syndrome (ARDS) selected from 1) lower respiratory symptoms of pulmonary congestion, tightness, shortness of breath and/or hypoxemia; 2) Extrapulmonary Systemic Hyperinflammation Syndrome (ESHS), and/or 3) another condition or symptom mediated by a severe hyper-immune or hyper-inflammatory response in the subject, including Pediatric Inflammatory Multisystem Syndrome (PIMS), vascular congestive and thrombotic conditions, Disseminated Intravascular Coagulation (DIC), thrombosis, stroke, and/or thrombocytopenia.

112. The method of claim 94, wherein the anti-inflammatory TPA compound is formulated and administered simultaneously with, or coordinately administered simultaneously or sequentially in a coordinate, multi-drug prophylactic or treatment protocol with, a secondary therapeutic or prophylactic drug or agent selected from: 1) a conventional anti-viral drug or agent; 2) a secondary anti-CSS drug or agent; 3) an anti-ARDS drug or agent, 4) an anti-PIMS drug or agent; 5) an anti-ESHS drug or agent; 6) an anti-DAD drug or agent; and/or 7) an anti-inflammatory, pro-immune, anti-cytopathic and/or pro-apoptotic drug or agent, and combinations thereof.

113. The method of claim 94, wherein the anti-inflammatory TPA compound is formulated and administered simultaneously with, or coordinately administered simultaneously or sequentially in a coordinate, multi-drug prophylactic or treatment protocol with, a secondary anti-viral drug or agent.

114. The method of claim 113, wherein the secondary anti-viral drug or agent is selected from: Abacavir, Acyclovir, Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine, and combinations thereof.

115. The method of claim 112, wherein the secondary drug or agent is an anti-ACE2 drug or agent.

116. The method of claim 112, wherein the secondary drug or agent is an anti-inflammatory drug or agent.

117. The method of claim 116, wherein the anti-inflammatory drug or agent is selected from non-steroidal anti-inflammatory drugs (NSAIDs).

118. The method of claim 117, wherein the NSAIDs include: aspirin, celecoxib (Celebrex), diclofenac (Cambia, Cataflam, Voltaren-XR, Zipsor, Zorvolex), diflunisal, etodolac, ibuprofen (Motrin, Advil), indomethacin (Indocin), celecoxib (Celebrex), piroxicam (Feldene), indomethacin (Indocin), meloxicam (Mobic Vivlodex), ketoprofen (Orudis, Ketoprofen ER, Oruvail, Actron), sulindac (Clinoril), diflunisal (Dolobid), nabumetone (Relafen), oxaprozin (Daypro), tolmetin (Tolmetin Sodium, Tolectin), salsalate (Disalcid), fenoprofen (Nalfon), flurbiprofen (Ansaid), ketorolac (Toradol), meclofenamate, mefenamic acid (Ponstel), and combinations thereof.

119. The method of claim 112, wherein the secondary drug or agent is a cytokine inhibitor drug or agent.

120. The method of claim 119, wherein the cytokine inhibitor drug or agent is effective to inhibit or lower induction, synthesis, activation and/or circulating level(s) of one or more pro-inflammatory cytokine targets selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.

121. The method of claim 112, wherein coordinate multi-drug therapy with the TPA compound and cytokine inhibitor drug is combinatorially effective to yield improved, additive, synergistic and/or potentiating therapeutic benefits (compared to benefits yielded by either drug/agent alone in a same dosage) for reducing induction, synthesis, activation and/or circulating level(s) of one or more pro-inflammatory cytokine targets that are hyper-elevated in association with SARS-COV-2 infection, COVID-19 disease, ARDS, SARS, CSS, PIMS, ESHS, DAD or another hyper-inflammatory condition mediated or exacerbated by SARS-COV-2 infection.

122. The method of claim 112, wherein the secondary drug or agent is an anti-IL-6 drug or biologic agent.

123. The method of claim 122, wherein the anti-IL-6 drug or biologic agent is an anti-IL-6 monoclonal antibody or Fab fragment, a soluble IL-6 receptor or receptor analog, or a cognate anti-IL-6 specific binding or deactivating domain thereof.

124. The method of claim 122, wherein the anti-IL-6 drug or biologic is selected from siltuximab, sarilumab (Kevzara), and tocilizumab (TCZ).

125. The method of claim 122, wherein the anti-IL-6 drug or biologic is an anti-IL-6 drug that blocks or inhibits IL-6 directly, or indirectly inhibits, lowers, or alters a pro-inflammatory activity of IL-6.

126. The method of claim 125, wherein the anti-IL-6 drug is andrographolide.

127. The method of claim 112, wherein the secondary drug or agent is a kinase modulating drug or agent.

128. The method of claim 127, wherein the kinase modulating drug or agent directly or indirectly inhibits, lowers, activates or alters an immune or inflammatory activity of one or more kinases involved in mediating or suppressing inflammatory responses, or regulating differentiation, proliferation, activation, inflammatory cytokine synthesis, and/or apoptotic activity of immune and/or inflammatory effector cells, including lymphocytes, monocyte/macrophage cells and/or neutrophils.

129. The method of claim 127, wherein the kinase modulator drug or agent modulates a mitogen activated protein kinase (MAPK), janus kinase (JAK) and/or protein kinase C (PKC).

130. The method of claim 127, wherein coordinate treatment of COVID-19 disease subjects with the TPA compound and kinase modulator drug clinically reduces one or more disease condition(s) or symptom(s) associated with severe SARS-COV-2 infection, including one or more condition(s) or symptom(s) associated with ARDS, SARS, CSS, PIMS, ESHS, and DAD.

131. The method of claim 112, wherein the secondary drug or agent is an anti-SARS-COV-2 vaccine agent.

132. The method of claim 112, wherein the secondary drug or agent is a composition comprising conditioned natural killer (NK) Cells.

133. The method of claim 112, wherein the secondary drug or agent is a composition comprising conditioned mesenchymal stem cells (MSCs).

134. The method of claim 112, wherein the secondary drug or agent is a recombinant Interferon.

135. The method of claim 112, wherein the secondary drug or agent is an intravenous formulated immunoglobulin.

136. The method of claim 112, wherein the secondary drug or agent comprises a SARS-COV-2-specific neutralizing antibody, Fab fragment or antibody binding domain.

137. The method of claim 112, wherein the secondary drug or agent comprises a C5a-specific antibody, Fab fragment or antibody binding domain.

138. The method of claim 112, wherein the secondary drug or agent is selected from Thalidomide, Fingolimod, anti-angiogenic drugs, hydroxychloroquine and glucocorticoids.

139. The method of claim 112, wherein the anti-inflammatory TPA compound and secondary drug or agent are combinatorially effective to mediate one or more significant clinical benefits relating to prevention and/or treatment of SARS-COV-2 viral infection and COVID-19 disease selected from: 1) preventing or reducing viral infection or titer in the upper respiratory tract; 2) preventing or reducing viral infection or titer the lower respiratory tract; 3) preventing or reducing viral infection or titer in non-respiratory, ACE-2 positive cell and tissues; 4) preventing or reducing viral attachment and entry into lung and other ACE-2 positive cells and tissues; 5) preventing or reducing viral replication in lung and other ACE-2 positive cell and tissues; and/or 7) preventing or reducing viral shedding from an upper respiratory tract of infected subjects.

140. An anti-viral composition or kit for use in human subjects presenting with medical risk factors for COVID-19 disease mediated by a SARS-COV-2 virus, or in patients presenting with a positive diagnosis for infection by the SARS-COV-2 virus, comprising an anti-viral effective TPA compound formulated or packaged with a secondary anti-viral drug or agent.

141. The anti-viral pharmaceutical composition or kit of claim 140, wherein the secondary anti-viral drug or agent is selected from: Abacavir, Acyclovir, Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine, and combinations thereof.

142. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from: 1) a conventional anti-viral drug or agent; 2) a secondary anti-ARDS drug or agent; 3) an anti-CSS drug or agent, 4) an anti-PIMS drug or agent; 5) an anti-ESHS drug or agent; 6) an anti-DAD drug or agent; and/or 7) an anti-inflammatory, pro-immune, anti-cytopathic and/or pro-apoptotic drug or agent; and combinations thereof.

143. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from non-steroidal anti-inflammatory drugs (NSAIDs).

144. The pharmaceutical composition of claim 143, wherein the NSAIDs include: aspirin, celecoxib (Celebrex), diclofenac (Cambia, Cataflam, Voltaren-XR, Zipsor, Zorvolex), diflunisal, etodolac, ibuprofen (Motrin, Advil), indomethacin (Indocin), celecoxib (Celebrex), piroxicam (Feldene), indomethacin (Indocin), meloxicam (Mobic Vivlodex), ketoprofen (Orudis, Ketoprofen ER, Oruvail, Actron), sulindac (Clinoril), diflunisal (Dolobid), nabumetone (Relafen), oxaprozin (Daypro), tolmetin (Tolmetin Sodium, Tolectin), salsalate (Disalcid), fenoprofen (Nalfon), flurbiprofen (Ansaid), ketorolac (Toradol), meclofenamate, mefenamic acid (Ponstel), and combinations thereof.

145. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from cytokine inhibitor drugs and agents.

146. The pharmaceutical composition or kit of claim 145, wherein the cytokine inhibitor drug or agent is effective to inhibit or lower induction, synthesis, activation and/or circulating level(s) of one or more pro-inflammatory cytokine targets selected from: (IL)-1B; IL-2; IL-6, IL-7; IL-8; IL-9; IL-10; fibroblast growth factor (FGF); granulocyte-macrophage colony stimulating factor (GM-CSF); IFNγ; granulocyte-colony stimulating factor (G-CSF); interferon-γ-inducible protein (IP10); monocyte chemoattractant protein (MCP1); macrophage inflammatory protein 1 alpha (MIP1A); platelet derived growth factor (PDGF); tumor necrosis factor (TNFα); and vascular endothelial growth factor (VEGF), and combinations thereof.

147. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from anti-IL-6 drugs and biologic agents.

148. The pharmaceutical composition or kit of claim 147, wherein the anti-IL-6 drug or biologic agent is an anti-IL-6 monoclonal antibody or Fab fragment, a soluble IL-6 receptor or receptor analog, or a cognate anti-IL-6 specific binding or deactivating domain thereof.

149. The pharmaceutical composition or kit of claim 147, wherein the anti-IL-6 drug or biologic is selected from siltuximab, sarilumab (Kevzara), and tocilizumab (TCZ).

150. The pharmaceutical composition or kit of claim 147, wherein the anti-IL-6 drug or biologic is an anti-IL-6 drug that blocks or inhibits IL-6 directly, or indirectly inhibits, lowers, or alters a pro-inflammatory activity of IL-6.

151. The pharmaceutical composition or kit of claim 150, wherein the anti-IL-6 drug is andrographolide.

152. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from kinase modulating drugs and agents.

153. The pharmaceutical composition or kit of claim 152, wherein the kinase modulating drug or agent directly or indirectly inhibits, lowers, activates or alters an immune or inflammatory activity of one or more kinases involved in mediating or suppressing inflammatory responses, or regulating differentiation, proliferation, activation, inflammatory cytokine synthesis, and/or apoptotic activity of immune and/or inflammatory effector cells, including lymphocytes, monocyte/macrophage cells and/or neutrophils.

154. The pharmaceutical composition or kit of claim 152, wherein the kinase modulator drug or agent modulates a mitogen activated protein kinase (MAPK), janus kinase (JAK) and/or protein kinase C (PKC).

155. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from sudden acute respiratory syndrome (SARS)-2 (SARS-COV-2 or COVID-19) coronavirus vaccine agents.

156. A pharmaceutical composition or kit for use in prevention or treatment of acute respiratory syndrome (ARDS) in a mammalian subject comprising an anti-ARDS effective TPA compound formulated or packaged with a secondary therapeutic or prophylactic drug or agent selected from: an interferon drug or agent; an intravenous formulated immunoglobulin; a SARS-CoV-2-specific neutralizing antibody, Fab fragment or antibody binding domain; a C5a-specific antibody, Fab fragment or antibody binding domain; thalidomide; fingolimod; anti-angiogenic drugs; hydroxychloroquine; and glucocorticoids, and combinations thereof.
